(19) United States
(12) Patent Application Publication
(10) Pub. No.: US 2025/0114092 A1
(43) Pub. Date: Apr. 10, 2025

(54) ANVIL COMPRISING AN ARRANGEMENT OF FORMING POCKETS PROXIMAL TO TISSUE STOP

(71) Applicant: Cilag GmbH International, Zug (CH)

(72) Inventors: Disha V. Estera, Cincinnati, OH (US); Michael J. Stokes, Cincinnati, OH (US); Anil K. Nalagatla, Mason, OH (US); Adam D. Hensel, Cincinnati, OH (US); Jason L. Harris, Lebanon, OH (US); Laura S. Downing, Cincinnati, OH (US); Shannon L. Jones, Cincinnati, OH (US)

(21) Appl. No.: 18/987,355

(22) Filed: Dec. 19, 2024

Related U.S. Application Data
(63) Continuation of application No. 17/503,879, filed on Oct. 18, 2021.

(51) Int. Cl. A61B 17/072

(52) U.S. Cl. CPC A61B 17/072; A61B 2017/07264; A61B 2017/07271; A61B 2017/07278; A61B 2017/07285

(57) ABSTRACT

A surgical stapling assembly is disclosed. The assembly can include a cartridge body having cavities defined therein forming a pattern of openings in the deck of the cartridge body. The pattern can include longitudinally-repeating clusters of cavity ends. The assembly can also include fasteners removably positioned in the cavities. The pattern of openings can comprise openings on a first side of the cartridge body comprising inner openings each defining an inner proximal-to-distal axis oriented parallel to the longitudinal axis, outer openings each defining an outer proximal-to-distal axis obliquely-oriented relative to the longitudinal axis at an outer angle, and intermediate openings each defining an intermediate proximal-to-distal axis obliquely-oriented relative to the longitudinal axis at an intermediate angle, wherein the intermediate angle is different than the outer angle. The assembly can include a sled having offset ramps and/or an anvil comprising an arrangement of forming pockets proximal to the tissue stop.

### PRIORITY

This application is a continuation of U.S. patent application Ser. No. 17/503,879, filed Oct. 18, 2021 and published as U.S. Pub. No. 2023/0121835 on Apr. 20, 2023, the disclosure of which is incorporated by reference herein in its entirety.

### BACKGROUND

The present invention relates to surgical instruments and, in various arrangements, to surgical stapling and cutting instruments and staple cartridges for use therewith that are designed to staple and cut tissue.

### SUMMARY

A surgical stapling assembly can comprise a first jaw and a second jaw. At least one of the first jaw and the second jaw can be movable between an open configuration and a closed configuration. The surgical stapling assembly can further comprise a fastener cartridge. The fastener cartridge can comprise a deck, a longitudinal slot defining a first cartridge side and a second cartridge side, staple cavities defined in the deck, staples removably stored in the staple cavities, and drivers movably supporting the staples. The staple cavities can comprise a longitudinally-oriented staple cavity and an obliquely-oriented staple cavity on the first cartridge side. The staples can comprise a longitudinally-oriented staple in the longitudinally-oriented staple cavity and an obliquely-oriented staple in the obliquely-oriented staple cavity. The drivers can comprise a first driver supporting the longitudinally-oriented staple and the obliquely-oriented staple. The surgical stapling assembly can further comprise an anvil comprising an anvil surface comprising staple forming pockets. The surgical stapling assembly can further comprise a proximal tissue stop defining a proximal-most tissue plane extending through the deck and the anvil surface when the first jaw and the second jaw are in the closed configuration. The longitudinally-oriented staple and the obliquely-oriented staple can be positioned proximal to the proximal-most tissue plane.

A surgical stapling assembly can comprise a fastener cartridge. The fastener cartridge can comprise a deck, a longitudinal slot defining a first cartridge side and a second cartridge side, staple cavities defined in the deck, staples removably stored in the staple cavities, drivers movably supporting the staples. The staple cavities can comprise a series of longitudinally-oriented staple cavities in a first row on the first cartridge side and a series of obliquely-oriented staple cavities in a second row on the first cartridge side. The staples can comprise a first longitudinally-oriented staple and a first obliquely-oriented staple. The drivers can comprise a triple driver movably supporting the first longitudinally-oriented staple and the first obliquely-oriented staple. The surgical stapling assembly can further comprise a proximal tissue stop defining a proximal-most tissue plane extending through the deck. The first longitudinally-oriented staple and the first obliquely-oriented staple can be positioned proximal to the proximal-most tissue plane.

A surgical stapling assembly can comprise a first jaw and a second jaw. At least one of the first jaw and the second jaw can be movable between an open configuration and a closed configuration. The surgical stapling assembly can further comprise a fastener cartridge. The fastener cartridge can comprise a deck, a longitudinal slot defining a first cartridge side and a second cartridge side, first staple cavities defined in the deck in a first cavity row on the first cartridge side, second staple cavities defined in the deck in a second cavity row on the first cartridge side, and staples removably stored in the first staple cavities and the second staple cavities. The surgical stapling assembly can further comprise a firing member configured to translate along the longitudinal slot to fire the staples, and an anvil. The anvil can comprise an anvil surface, first forming pockets defined in the anvil surface, and second forming pockets defined in the anvil surface. The first forming pockets can be configured to form the staples removably stored in the first staple cavities into a planar staple configuration. The second forming pockets can be configured to form the staples removably stored in the second staple cavities into a nonplanar staple configuration. The surgical stapling assembly can further comprise a proximal tissue stop defining a proximal-most tissue plane extending through the deck and the anvil surface. At least one first forming pocket and at least one second forming pocket can be proximal to the proximal-most tissue plane.

### BRIEF DESCRIPTION OF THE DRAWINGS

The various aspects described herein, both as to organization and methods of operation, together with further objects and advantages thereof, may best be understood by reference to the following description, taken in conjunction with the accompanying drawings as follows.

FIG. 1 is a perspective view of a surgical stapling assembly including a fastener cartridge assembly installed therein, according to various aspects of the present disclosure.

FIG. 2 is a perspective view of the fastener cartridge assembly of FIG. 1, according to various aspects of the present disclosure.

FIG. 3 is an exploded perspective view of the fastener cartridge assembly of FIG. 2 depicting the cartridge body, sled, drivers, and staples, according to various aspects of the present disclosure.

FIG. 4 is an elevation view of the fastener cartridge assembly of FIG. 2, according to various aspects of the present disclosure.

FIG. 5 is another perspective view of the fastener cartridge assembly of FIG. 2, depicting an underside of the cartridge body, sled, and drivers, according to various aspects of the present disclosure.

FIG. 6 is a perspective view of a proximal portion of the fastener cartridge assembly of FIG. 2, according to various aspects of the present disclosure.

FIG. 7 is another perspective view of the proximal portion of the fastener cartridge assembly of FIG. 2, according to various aspects of the present disclosure.

FIG. 8 is an elevation view of the fastener cartridge assembly of FIG. 2 mid-firing stroke depicting the staples being ejected from the cartridge body during a firing stroke, according to various aspects of the present disclosure.

FIG. 9 is a perspective partial cross-sectional view of the fastener cartridge assembly of FIG. 2 mid-firing stroke as shown in FIG. 8, according to various aspects of the present disclosure.

FIG. 10 is a perspective cross-sectional view of the fastener cartridge assembly of FIG. 2 depicting drivers and staples in a fired configuration, according to various aspects of the present disclosure.

FIG. 11 is an elevation view of a proximal portion of the fastener cartridge assembly of FIG. 2 with the staples removed for illustrative purposes, according to various aspects of the present disclosure.

FIG. 12 is another elevation view of the proximal portion of the fastener cartridge assembly of FIG. 2 depicting the underside of the fastener cartridge assembly and with the staples and drivers removed for illustrative purposes, according to various aspects of the present disclosure.

FIG. 13 is an elevation view of a pair of drivers from the fastener cartridge assembly of FIG. 2 depicting a right-side driver and a left-side driver positioned on opposite sides of the cartridge body, according to various aspects of the present disclosure.

FIG. 14 is another elevation view of the pair of drivers of FIG. 13, according to various aspects of the present disclosure.

FIG. 15 is an elevation view of the left-side driver of FIG. 13, according to various aspects of the present disclosure.

FIG. 16 is a perspective view of the left-side driver of FIG. 13, according to various aspects of the present disclosure.

FIG. 17 is another perspective view of the left-side driver of FIG. 13, according to various aspects of the present disclosure.

FIG. 18 is a bottom perspective view of a portion of the fastener cartridge assembly of FIG. 2, according to various aspects of the present disclosure.

FIG. 19 is an elevation view of a portion of the fastener cartridge assembly of FIG. 2, according to various aspects of the present disclosure.

FIGS. 20-22 are elevation views of a portion of the fastener cartridge assembly of FIG. 2 depicting various dimensional relationships thereof and FIG. 22A is a detail view of FIG. 22, according to various aspects of the present disclosure.

FIG. 23 is an elevation view of the left-side driver of FIG. 13 depicting various dimensional relationships thereof, according to various aspects of the present disclosure.

FIG. 24 is an elevational view of the sled and drivers (or portions of drivers) from the fastener cartridge assembly of FIG. 2, according to various aspects of the present disclosure.

FIG. 25 is an elevation views of a portion of the fastener cartridge assembly of FIG. 2 depicting various dimensional relationships thereof, according to various aspects of the present disclosure.

FIG. 26 is a perspective view of the anvil of FIG. 2, according to various aspects of the present disclosure.

FIG. 27 is a perspective view of a portion of the anvil of FIG. 26, according to various aspects of the present disclosure.

FIG. 28 is an elevation view of a portion of the anvil of FIG. 26 and FIG. 28A is a detail view of FIG. 28, according to various aspects of the present disclosure.

FIG. 29 is an elevation view of an inner staple formed by an inner forming pocket of the anvil of FIG. 26, according to various aspects of the present disclosure.

FIG. 30 is an elevation view of an intermediate staple formed by an intermediate forming pocket of the anvil of FIG. 26, according to various aspects of the present disclosure.

FIG. 31 is an elevation view of an outer staple formed by an outer forming pocket of the anvil of FIG. 26, according to various aspects of the present disclosure.

FIG. 32 is an example pressure distribution schematic for a portion of a staple line comprised of longitudinally-oriented planar staples in three adjacent rows, according to various aspects of the present disclosure.

FIG. 33 is an example pressure distribution schematic for a portion of a staple line formed by the surgical stapling assembly 200 of FIG. 1 and including staples at different angular orientations row-to-row, according to various aspects of the present disclosure.

FIG. 34 is an example strain contour schematic for pressure and stretch loading conditions in a portion of a staple line comprised of longitudinally-oriented planar staples in three adjacent rows, according to various aspects of the present disclosure.

FIG. 35 is an example strain contour schematic for pressure and stretch loading conditions in a portion of a staple line formed by the surgical stapling assembly 200 of FIG. 1 and including staples at different angular orientations row-to-row, according to various aspects of the present disclosure.

FIG. 36 is a perspective view of a portion of an anvil, according to various aspects of the present disclosure.

FIG. 37 is an elevation view of a portion of the anvil of FIG. 36, according to various aspects of the present disclosure.

FIG. 37A is a detail view of FIG. 37, according to various aspects of the present disclosure.

FIG. 38 is an elevation view of a staple forming pocket of the anvil of FIG. 36, according to various aspects of the present disclosure.

FIG. 39 is an elevation cross-sectional view of the staple forming pocket of FIG. 38 taken along plane 39-39, according to various aspects of the present disclosure.

FIG. 40 is an elevation cross-sectional view of the staple forming pocket of FIG. 38 taken along plane 40-40, according to various aspects of the present disclosure.

FIG. 41 is an elevation cross-sectional view of the staple forming pocket of FIG. 38 taken along plane 41-41, according to various aspects of the present disclosure.

FIG. 42 is a perspective view of a fastener cartridge assembly, according to various aspects of the present disclosure.

FIG. 43 is a top elevation view of a proximal portion of the fastener cartridge assembly of FIG. 42 depicting first drivers and second drivers positioned therein, according to various aspects of the present disclosure.

FIG. 44 is a bottom elevation view of the proximal portion of the fastener cartridge assembly of FIG. 42 depicting the first drivers and the second drivers positioned therein, according to various aspects of the present disclosure.

FIG. 45 is a perspective view of one of the first drivers in FIGS. 43 and 44 from the fastener cartridge assembly of FIG. 42, according to various aspects of the present disclosure.

FIG. 46 is a perspective view of the first driver of FIG. 45, according to various aspects of the present disclosure.

FIG. 47 is an elevation view of the first driver of FIG. 45, according to various aspects of the present disclosure.

FIG. 48 is a perspective view of a fastener cartridge assembly, according to various aspects of the present disclosure.

FIG. 49 is a top elevation view of a proximal portion of the fastener cartridge assembly of FIG. 48 depicting first drivers and second drivers positioned therein, according to various aspects of the present disclosure.

FIG. 50 is a bottom elevation view of the proximal portion of the fastener cartridge assembly of FIG. 48 depicting the first drivers and the second drivers positioned therein, according to various aspects of the present disclosure.

FIG. 51 is a first perspective view of a proximal portion of the fastener cartridge assembly of FIG. 48, according to various aspects of the present disclosure.

FIG. 52 is a second perspective view of one of the first drivers in FIGS. 49 and 50 from the fastener cartridge assembly of FIG. 48, according to various aspects of the present disclosure.

FIG. 53 is a perspective view of the first driver of FIG. 52, according to various aspects of the present disclosure.

FIG. 54 is a side elevation view of the first driver of FIG. 52, according to various aspects of the present disclosure.

FIG. 55 is a top elevation view of the first driver of FIG. 52, according to various aspects of the present disclosure.

FIG. 56 is a bottom elevation view of the first driver of FIG. 52, according to various aspects of the present disclosure.

FIG. 57 is a perspective view of a fastener cartridge assembly, according to various aspects of the present disclosure.

FIG. 58 is an elevation view of a portion of the fastener cartridge assembly of FIG. 57, according to various aspects of the present disclosure.

FIG. 59 is a perspective view of a driver for the fastener cartridge assembly of FIG. 57 depicting a pair of crossed staples supported thereon, according to various aspects of the present disclosure.

FIG. 60 is a perspective view of a fastener cartridge assembly, according to various aspects of the present disclosure.

FIG. 61 is a top elevation view of a proximal portion of the fastener cartridge assembly of FIG. 60, according to various aspects of the present disclosure.

FIG. 62 is a partial cutaway perspective view of a portion of the fastener cartridge assembly of FIG. 60 depicting a firing motion of a firing member thereof, according to various aspects of the present disclosure.

FIG. 63 is a perspective view of a series of drivers and staples from the fastener cartridge assembly of FIG. 60, according to various aspects of the present disclosure.

FIG. 64 is a perspective view of a driver and pair of crossed staples supported thereon from the fastener cartridge assembly of FIG. 60, according to various aspects of the present disclosure.

FIG. 65 is a top elevation view of the series of drivers and staples of FIG. 63, according to various aspects of the present disclosure.

FIG. 66 is a top elevation view of the driver and the pair of crossed staples of FIG. 64, according to various aspects of the present disclosure.

FIG. 67 is a perspective view of an anvil for use with the fastener cartridge assembly of FIG. 60, according to various aspects of the present disclosure.

FIG. 68 is an elevation view of a proximal portion of the anvil of FIG. 67, according to various aspects of the present disclosure.

FIG. 69 is a perspective view of an interchangeable surgical tool assembly embodiment operably coupled to a handle assembly embodiment, according to various aspects of the present disclosure.

FIG. 70 is an exploded assembly view of portions of the handle assembly and interchangeable surgical tool assembly of FIG. 69, according to various aspects of the present disclosure.

Corresponding reference characters indicate corresponding parts throughout the several views. The exemplifications set out herein illustrate various embodiments of the invention, in one form, and such exemplifications are not to be construed as limiting the scope of the invention in any manner.

### DETAILED DESCRIPTION

The following U.S. Patent Applications are filed on Oct. 18, 2021 and are incorporated by reference herein in their respective entireties:
U.S. Patent Application titled SURGICAL STAPLING ASSEMBLY WITH OFFSET RAMPED DRIVE SURFACES; U.S. patent application Ser. No. 17/503,834, published as U.S. Pub. No. 2023/0132138 on Apr. 27, 2023;
SURGICAL STAPLING ASSEMBLY HAVING LONGITUDINALLY-REPEATING STAPLE LEG CLUSTERS; U.S. patent application Ser. No. 17/503,847, published as U.S. Pub. No. 2023/0120983 on Apr. 20, 2023; and
ROW-TO-ROW STAPLE ARRAY VARIATIONS; U.S. patent application Ser. No. 17/503,857, published as U.S. Pub. No. 2023/0121658 on Apr. 20, 2023.

Certain exemplary embodiments will now be described to provide an overall understanding of the principles of the structure, function, manufacture, and use of the devices and methods disclosed herein. One or more examples of these exemplary embodiments are illustrated in the accompanying drawings. Those of ordinary skill in the art will understand that the devices and methods specifically described herein and illustrated in the accompanying drawings are non-limiting exemplary embodiments and that the scope of the various exemplary embodiments of the present invention is defined solely by the claims. The features illustrated or described in connection with one exemplary embodiment may be combined with the features of other exemplary embodiments. Such modifications and variations are intended to be included within the scope of the present invention.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a surgical system, device, or apparatus that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Various exemplary devices and methods are provided for performing laparoscopic and minimally invasive surgical procedures. However, the reader will readily appreciate that the various methods and devices disclosed herein can be used in numerous surgical procedures and applications including, for example, in connection with open surgical procedures. As the present Detailed Description proceeds, the reader will further appreciate that the various instruments disclosed herein can be inserted into a body in any way, such as through a natural orifice, through an incision or puncture hole formed in tissue, etc. The working portions or end effector portions of the instruments can be inserted directly into a patient's body or can be inserted through an access device that has a working channel through which the end effector and elongate shaft of a surgical instrument can be advanced.

A surgical stapling system can comprise a shaft and an end effector extending from the shaft. The end effector comprises a first jaw and a second jaw. The first jaw comprises a staple cartridge. The staple cartridge is insertable into and removable from the first jaw; however, other embodiments are envisioned in which a staple cartridge is not removable from, or at least readily replaceable from, the first jaw. The second jaw comprises an anvil configured to deform staples ejected from the staple cartridge. The second jaw is pivotable relative to the first jaw about a closure axis; however, other embodiments are envisioned in which the first jaw is pivotable relative to the second jaw. The surgical stapling system further comprises an articulation joint configured to permit the end effector to be rotated, or articulated, relative to the shaft. The end effector is rotatable about an articulation axis extending through the articulation joint. Other embodiments are envisioned which do not include an articulation joint.

The staple cartridge comprises a cartridge body. The cartridge body includes a proximal end, a distal end, and a deck extending between the proximal end and the distal end. In use, the staple cartridge is positioned on a first side of the tissue to be stapled and the anvil is positioned on a second side of the tissue. The anvil is moved toward the staple cartridge to compress and clamp the tissue against the deck. Thereafter, staples removably stored in the cartridge body can be deployed into the tissue. The cartridge body includes staple cavities defined therein wherein staples are removably stored in the staple cavities. The staple cavities are arranged in six longitudinal rows. Three rows of staple cavities are positioned on a first side of a longitudinal slot and three rows of staple cavities are positioned on a second side of the longitudinal slot. Other arrangements of staple cavities and staples may be possible.

The staples are supported by staple drivers in the cartridge body. The drivers are movable between a first, or unfired position, and a second, or fired, position to eject the staples from the staple cavities. The drivers are retained in the cartridge body by a retainer which extends around the bottom of the cartridge body and includes resilient members configured to grip the cartridge body and hold the retainer to the cartridge body. The drivers are movable between their unfired positions and their fired positions by a sled. The sled is movable between a proximal position adjacent the proximal end of the cartridge body and a distal position adjacent the distal end of the cartridge body. The sled comprises a plurality of ramped surfaces configured to slide under the drivers and lift the drivers, and the staples supported thereon, toward the anvil.

Further to the above, the sled is moved distally by a firing member. The firing member is configured to contact the sled and push the sled toward the distal end. The longitudinal slot defined in the cartridge body is configured to receive the firing member. The anvil also includes a slot configured to receive the firing member. The firing member further comprises a first cam which engages the first jaw and a second cam which engages the second jaw. As the firing member is advanced distally, the first cam and the second cam can control the distance, or tissue gap, between the deck of the staple cartridge and the anvil. The firing member also comprises a knife configured to incise the tissue captured intermediate the staple cartridge and the anvil. It is desirable for the knife to be positioned at least partially proximal to the ramped surfaces such that the staples are ejected ahead of the knife.

Firing a surgical stapling assembly can involve advancing a firing member in a first direction to move the fasteners in a second direction. For example, a firing member can be advanced distally along a longitudinal axis of a fastener cartridge during a firing stroke. The firing member, or sled, can include longitudinal rails, which are configured to engage corresponding ramped surfaces on staple-supporting drivers. Movement of the rails along the ramped surfaces can convert the longitudinal movement of the firing member to a transverse, lifting movement of the drivers. As the drivers are lifted vertically in the fastener cartridge, the fasteners can be pushed upward and out of the fastener cartridge.

The firing member is configured to transmit significant forces to the drivers during the firing stroke. These forces are sufficient to eject the fasteners from cavities in the fastener cartridge and into tissue clamped between jaws of a surgical end effector. The geometry of the drivers relative to the cartridge body can facilitate a smooth firing motion. Conversely, ill-fitting drivers relative to the cartridge body can be prone to torqueing or twisting during the firing motion, which can result in misfired fasteners and/or jamming of the surgical end effector.

The arrangement of the staples in a resultant staple line is also an important consideration. Certain staple line geometries can better accommodate stretching or movement of the stapled tissue. For example, staple lines having obliquely-oriented staples relative to a centerline, or cutline, can better facilitate tissue extension in certain instances. For example, the stapled tissue may be able to stretch and flex more when the staple line includes obliquely-oriented staples. However, obliquely-oriented staples present additional challenges regarding nesting obliquely-oriented staples in a fastener cartridge, nesting obliquely-oriented forming pockets in an anvil that aligned with the obliquely-oriented staples, balancing the torque during a firing stroke, and supporting the staple during the firing motion, and among other things.

In certain aspects of the present disclosure, a resultant staple line can include staples oriented at increasing angles outward from the centerline. For example, staples in an intermediate staple row can be positioned at a greater angle relative to the centerline than staples in an inner staple row and at a lesser angle relative to the centerline than staples in an outer staple row. Additionally or alternatively, the resultant staple line can include a space between adjacent staples in the same row that increases outward from the centerline. For examples, staples in an intermediate row can be spaced apart more than staples in the inner staple row and less than staples in the outer staple row.

In certain instances, the resultant staple line can include both longitudinally-oriented staples and obliquely-oriented staples. For example, a first row can include longitudinally-oriented staples, and a second row can include obliquely-oriented staples. Longitudinally-oriented staples can be parallel to the longitudinal axis and/or firing path through the cartridge body. In various instances, the longitudinally-oriented staples can be positioned adjacent to the cutline, which can advance sealing and hemostasis along the cutline. For example, the innermost row of staples (on one or both sides of the cutline) can include longitudinally-oriented staples, and the outer row or rows of staples (on one or both sides of the cutline) can include obliquely-oriented staples.

Additionally or alternatively, the resultant staple line can also include both planar staples (e.g. 2-D staples) and non-planar staples (e.g. 3-D staples). For example, a first row can include planar staples, and a second row can include non-planar staples. Non-planar staples can form a modified B-shape in which the staple legs diverge away from the centerline and base of the staple. In various instances, the planar staples can be positioned adjacent to the cutline, which can advance sealing and hemostasis along the cutline. For example, the innermost row of staples (on one or both sides of the cutline) can include planar staples, and the outer row or rows of staples (on one or both sides of the cutline) can include non-planar staples. In certain instances, the planar staples can be aligned with the longitudinal axis and the non-planar staples can be obliquely-oriented relative to the longitudinal axis.

The improved fastener cartridges and staple patterns further described herein can better balance the staple drivers, prevent driver roll, assure tight fits between the drivers and the cavities, preserve the wall thicknesses of the cartridge body and/or protect the integrity of the driver ramps against the sled in certain instances. These improvements and others are further described herein.

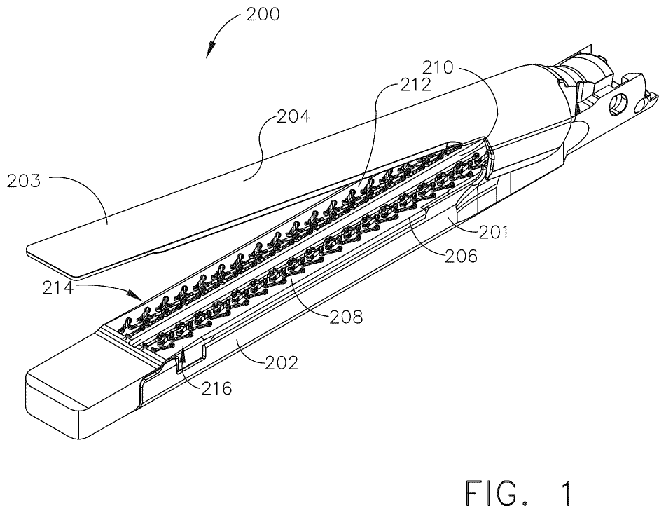

FIG. 1 depicts a surgical stapling assembly 200 in an open configuration. More specifically, the surgical stapling assembly includes a first jaw 201 and a second jaw 203 configured to move between the open position and a closed position. In the closed position, the jaws 201 and 203 can close on tissue to grip, clamp, manipulate, and otherwise effect the tissue. The first jaw 201 is configured to pivot relative to the second jaw 203. In other instances, the second jaw 203 can be configured to pivot relative to the first jaw 201. One jaw 201 or 203 can be fixed and the other jaw 201 or 203 can pivot in certain instances. In other instances, both jaws 201 and 203 can pivot to move the surgical stapling assembly between the open configuration and the closed configuration.

The surgical stapling assembly 200 further includes an elongate channel 202 and an anvil 204. The elongate channel 202 is configured to receive a fastener cartridge assembly 206. The fastener cartridge assembly 206 can be removably positioned in the elongate channel 202. For example, the surgical stapling assembly 200 can be a reusable component and the fastener cartridge assembly 206 can be a replaceable component, which can be replaced with another fastener cartridge assembly 206 for a subsequent firing and/or subsequent use. An anvil 204 opposes the fastener cartridge assembly 206. The anvil is configured to form fasteners ejected from the fastener cartridge assembly during a firing stroke.

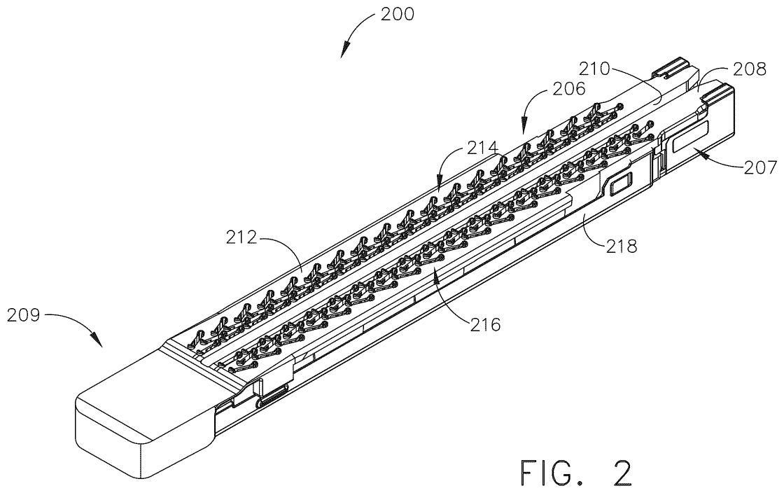

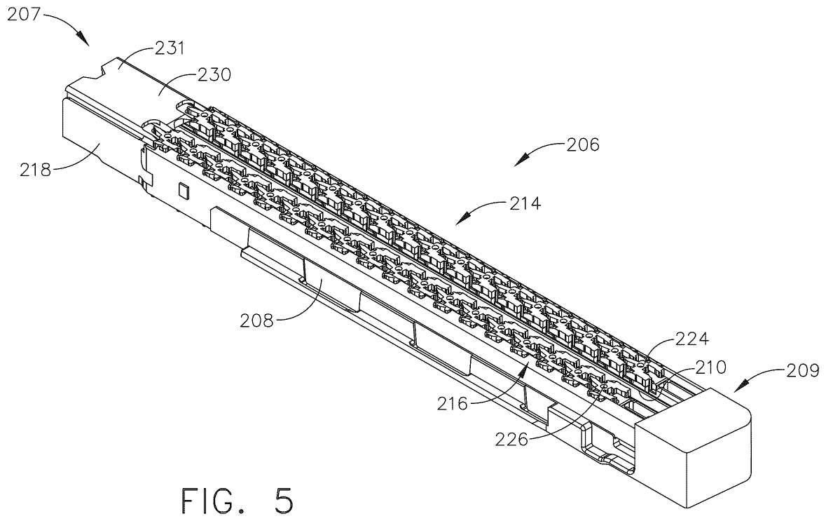

Referring primarily to FIGS. 2-6, the fastener cartridge assembly 206 includes a cartridge body 208 having a longitudinal knife slot 210 defined at least partially through the cartridge body 208 from a proximal end 207 of the cartridge body 208 toward the distal end 209 of the cartridge body 208. The longitudinal knife slot 210 is configured to receive a firing member therethrough. An upright portion of the firing member can include a cutting edge. In certain instances, the cutting edge is configured to translate through the fastener cartridge assembly 206 during a firing stroke to sever tissue clamped between the jaws 201 and 203.

The cartridge body 208 further includes a tissue-supporting deck 212. Cavities 214 are defined into the tissue-supporting deck 212 forming openings therein. The cavities 214 are configured to receive fasteners and guide the fasteners toward and into tissue during a firing motion. The cavities 214 are arranged in a plurality of rows 216.

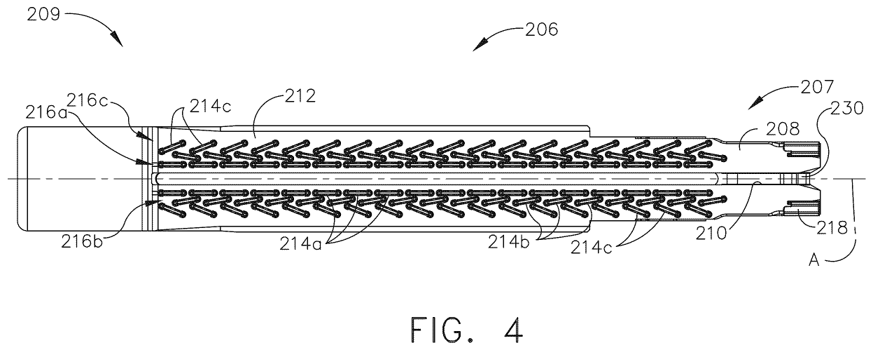

Referring primarily to FIG. 4, the cartridge body 208 includes three rows 216 on both sides of the longitudinal knife slot 210. The rows includes an inner row 216a comprised of inner cavities 214a, an intermediate row 216b comprised of intermediate cavities 214b, and an outer row 216c comprised of outer cavities 214c.

In other instances, the cartridge body 208 can include additional rows of cavities or fewer rows of cavities on one or both sides of the longitudinal knife slot 210. In certain instances, the fastener cartridge assembly 206 can be symmetrical about a longitudinal axis A and the rows of cavities can be symmetrical about the longitudinal axis A. In other instances, the rows of cavities can be asymmetrical about the longitudinal axis A.

The inner cavities 214a in cartridge body 208 are longitudinally-oriented relative to the longitudinal axis A. The intermediate cavities 214b and the outer cavities 214c are obliquely-oriented relative to the longitudinal axis A. The intermediate cavities 214b are angled away from the longitudinal axis A, and the outer cavities are angled toward the longitudinal axis A. In such instances, the longitudinal alignment of the inner cavities 214a and the angled orientations of the intermediate cavities 214b and the outer cavities 214c allow the intermediate cavities to nest closely between the inner cavities 214a and the outer cavities 214c. The nested arrangement of staple cavities positioned at different angular orientations can provide a compact arrangement of staple cavities and corresponding staples. Various exemplary dimensions, which enable to the array of drivers, staples, forming-pockets, and so on to fit within the small form factor of a fastener cartridge assembly for minimally-invasive surgeries, for example.

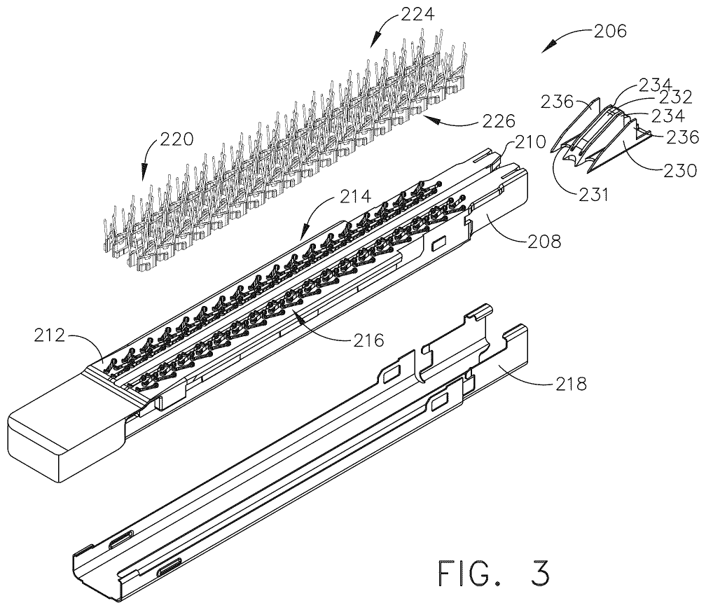

Referring primarily to FIG. 3, the fastener cartridge assembly 206 further comprises a cartridge pan 218 configured to encase a portion of the cartridge body 208. Staples 220 are removably positioned in the cavities 214 in the cartridge body 208. The staples 220 can be V-shaped staples having a linear base, a first leg extending from a first end of the linear base, and a second leg extending from the second end of the linear base. The first leg and the second leg splay laterally outward to engage the inside walls of the cavities 214. The staples 220 can be held in the cavities 214 prior to a firing stroke with a friction-fit connection between the legs and the inside walls of the cavities 214.

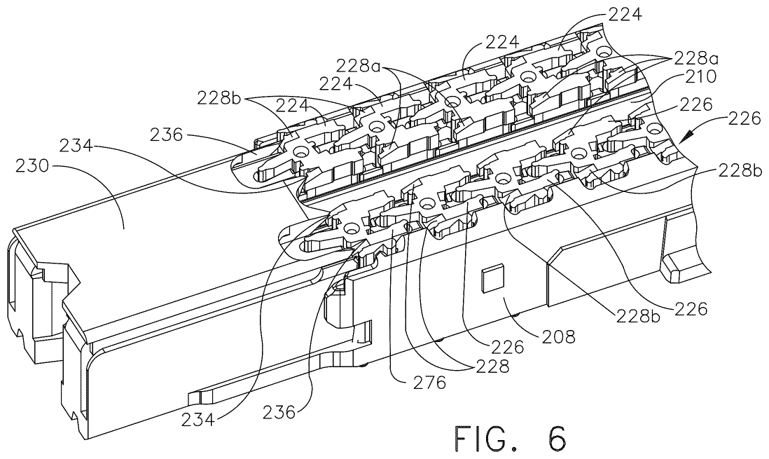

The staples 220 are movably supported by drivers 224, 226 in the cartridge body 208. The drivers 224, 226 are triple drivers. Each driver 224, 226 includes three cradles and is configured to support a staple 220 in the inner row 216a, a staple 220 in the intermediate row 216b, and a staple 220 in the outer row 216c. To support the staples 220 oriented at different angles across multiple rows 216, the triple drivers 224, 226 include support columns comprising cradles oriented at different angles, connectors between the support columns, and ramps 228 (i.e. inner ramps 228a and outer ramps 228b) (FIG. 6) on the underside configured to be driven by a sled 230 during the firing stroke. The drivers 224, 226 are first drivers 224 on a first side (e.g. right side) of the cartridge body 208, and second drivers 226 on a second side (e.g. left side) of the cartridge body 208. The first driver 224 is a mirror image of the second driver 226 owing to the symmetrical arrangement of the cavities 214 and the staples 220 on either side of the longitudinal axis A. The cartridge pan 218 is configured to retain the drivers 224, 226 in the cartridge body 208.

The fastener cartridge assembly 206 also includes the sled 230 configured to translate distally during a firing motion to engage the drivers 224, 226 and lift the drivers 224, 226 to eject the fasteners 220 from the cartridge body 208. Referring primarily to FIG. 3, the sled 230 is a four-rail sled having a connecting flange 231, a central upright 232 configured to move through the longitudinal knife slot 210, a pair of inner rails 234, and a pair of outer rails 236. An inner rail 234 and an outer rail 236 are configured to driving engage an inner ramp 228a and an outer ramp 228b, respectively, of each driver 224, 226 to lift the drivers 224, 226 toward the deck 212.

As further described herein, the sled 230 can be comprised of metal, which can enable the inner rails 234 and the outer rails 236 to be narrower while transmitting sufficient forces to the drivers 224, 226 without buckling or otherwise deforming under the load. Narrower rails correspond to narrower rail channels through the cartridge body 208 such that the cartridge body walls can be widened to better guide the drivers 224, 226 and staples 220 thereon during the firing stroke.

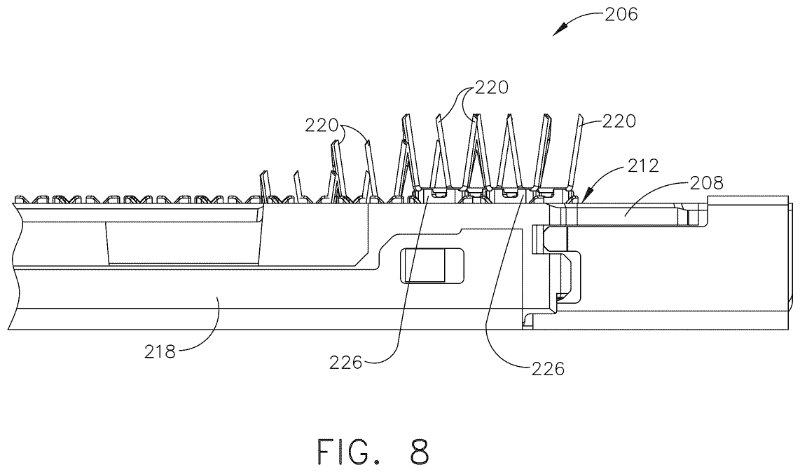

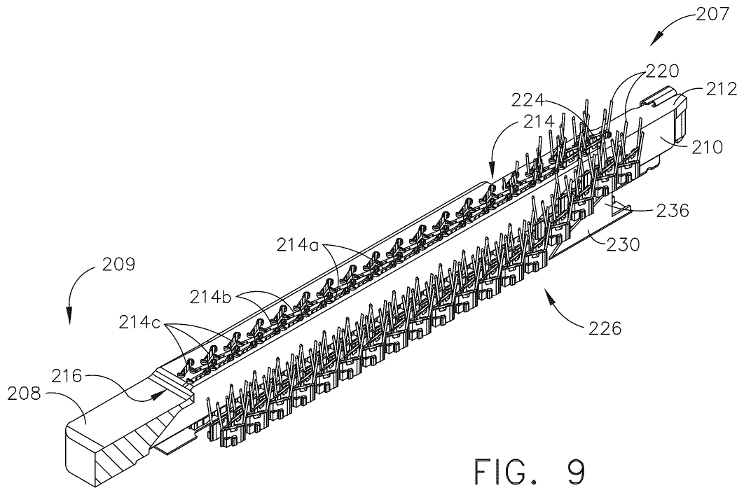

FIGS. 8 and 9 depict the fastener cartridge assembly 206 in which the sled 230 is advanced distally from a proximal-most position to an intermediate position mid-firing stroke. The inner rails 234 and the outer rails 236 are engaged with multiple adjacent drivers 224, 226 during the firing stroke and the proximal-most drivers 224, 226 have already been driven upward through the cavities 214 to fire the proximal-most staples 220 out of the cartridge body 208. The drivers 224, 226 and the staples 220 supported thereon are progressively driven and fired from the proximal end 207 to the distal end 209 of the cartridge body 208 during the firing stroke. The reader will appreciate that the sequential proximal-to-distal driving of drivers 224, 226 and firing of staples 220 is configured to seal tissue adjacent to a cutline in close proximity to the cutting. For example, the staples 220 can be configured to engage and seal tissue as the cutting edge transects the intermediate tissue. In certain instances, transection can immediately following stapling.

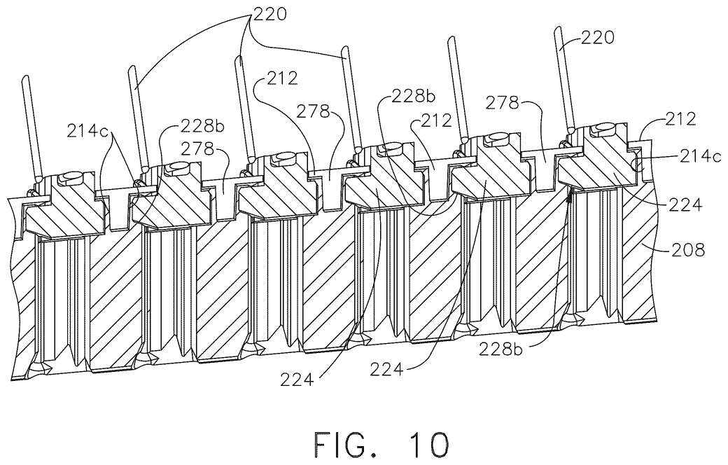

Upon completion of the firing stroke, the drivers 224, 226 can be driven upward in the cavities 214 to lift the staples 220 out of the cartridge body 208. Referring primarily to FIG. 10, the drivers 224 have been lifted to their fully fired positions in which the staple-supporting cradles thereof are positioned outside the cartridge body 208. In such instances, the drivers 224 have been overdriven. Overdrive of the drivers 224 allows the bases of the staples 220 to clear the deck 212 and completely disengage the fastener cartridge assembly 206 during the firing stroke.

Referring now to FIGS. 11 and 12, a proximal portion of the fastener cartridge assembly 206 is shown with the sled 230 in an unfired, proximal position. The rows 216 of cavities 214 are symmetric about the longitudinal knife slot 210. Each cavity 214 includes a proximal end 250 and a distal end 252. The first cavities 214a in the first rows 216a (and the deck openings thereof) are longitudinally-oriented relative to the longitudinal axis A. For example, each first cavities 214a can define a first proximal-to-distal axis PD1 between its proximal end 250 and its distal end 252. The first proximal-to-distal axes PD1 are parallel to the longitudinal axis A. The first cavities 214a are longitudinally-spaced apart a first length LI. The first length L1 is defined in a longitudinal direction from the distal end 252 of one first cavity 214a to the proximal end 250 of the adjacent first cavity 214a in the first row 216a.

The second cavities 214b in the second rows 216b (and the deck openings thereof) are obliquely-oriented relative to the longitudinal axis A. For example, each second cavity 214b can define a second proximal-to-distal axis PD2 between its proximal end 250 and its distal end 252. The second proximal-to-distal axes PD2 are obliquely-oriented relative to the longitudinal axis A at a second cavity angle θ2. The second proximal-to-distal axes PD2 are angled away from the longitudinal axis A. The second cavities 214b are longitudinally-spaced apart a second length L2. The second length L2 is defined in a longitudinal direction from the distal end 252 of one second cavity 214b to the proximal end 250 of the adjacent second cavity 214b in the second row 216b.

The third cavities 214c in the third rows 216c (and the deck openings thereof) are obliquely-oriented relative to the longitudinal axis A. For example, each third cavity 214c can define a third proximal-to-distal axis PD3 between its proximal end 250 and its distal end 252. The third proximal-to-distal axes PD3 are obliquely-oriented relative to the longitudinal axis A at a third cavity angle θ3. The third proximal-to-distal axes PD3 are angled toward the longitudinal axis A. The third cavities 214c are longitudinally-spaced apart a third length L3. The third length L3 is defined in a longitudinal direction from the distal end 252 of one third cavity 214c to the proximal end 250 of the adjacent third cavity 214c in the third row 216c.

The third cavity angle θ3 is greater than the second cavity angle θ2. For example, the second cavity angle θ2 can be approximately 10 degrees, and the third cavity angle θ3 can be approximately 21.5 degrees. Both the second and third cavities angles θ2, θ3 are greater than the angle at which the first cavities 214a are oriented relative to the longitudinal axis. For example, where the first cavities 214a are parallel to the longitudinal axis A, the angle of the first cavities 214a relative to the longitudinal axis A can be zero. In various instances, the angular orientation of the staple cavities 214 can define an increasing gradient from the centerline outbound toward the lateral sides of the cartridge body 208. For example, where the fastener cartridge assembly 206 includes a fourth row 216 of cavities 214 on one (or both) sides of the longitudinal knife slot 210, the fourth row 216 could be oriented at a greater angle than second cavity angles θ3, for example.

The third cavity angle θ3 can be sufficiently angled such that the sled 230 can pass completely under the staples 220 in the outer row 216c while still providing sufficient space for a cartridge outside wall to vertically support the outside portion of the driver 224, 226 and the staple 220 thereon. For example, the third cavity angle θ3 can be greater than or less than 21.5 degrees in certain instances while still maintaining a minimum outside wall thickness that is sufficient for vertical support and suitable for injection molding (e.g. around a 0.01 inch minimum wall thickness).

The third length L3 is greater than the second length L2 and the first length L1. In various instances, the first length L1 is less than the second length L2 such that the longitudinal lengths between adjacent cavities 214 increases laterally outboard from the longitudinal axis A. Stated differently, as the angular orientation of the cavities 214 increases, the longitudinal length between adjacent cavities 214 also increases.

Referring primarily now to FIG. 21, the end-to-end distance between each staple cavity corresponds to a staple gap between the distal end of one staple 220 and the proximal end of an adjacent staple in the same row. The inner cavities 214a define a first gap G1, the intermediate cavities 214b define a second gap G2, and the outer cavities 214c define a third gap G3. The third gap G3 is greater than the second gap G2 and the first gap G1. In various instances, the first gap G1 is less than the second gap G2 such that gaps between adjacent staples 220 in a staple line increase laterally outboard from the longitudinal axis A. Stated differently, as the angular orientation of the staples 220 increases, the staple gaps between adjacent staples 220 also increase. For example, the first gap G1 can be approximately 0.025 inches, the second gap G2 can be approximately 0.0303 inches, and the third gap G3 can be approximately 0.0454 inches.

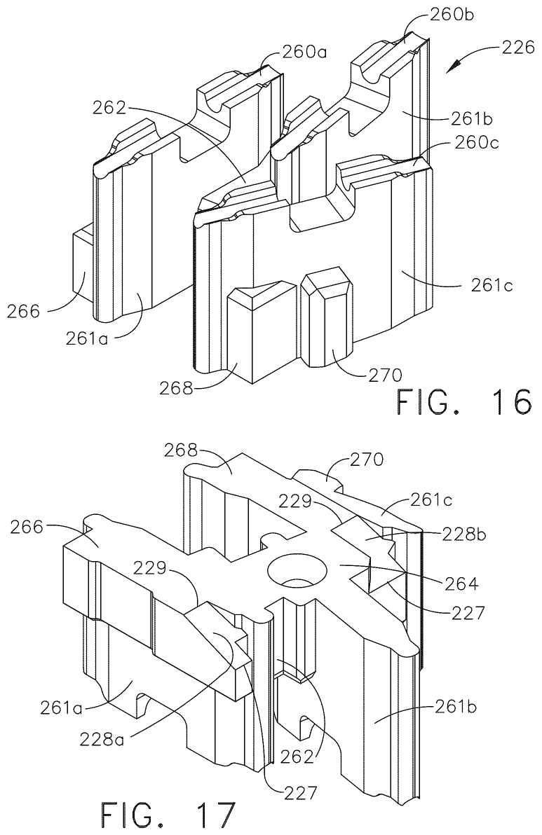

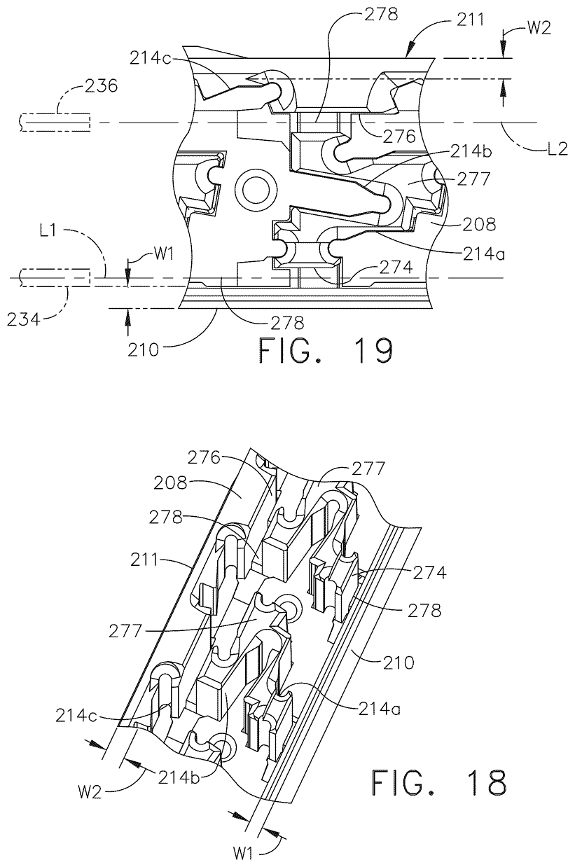

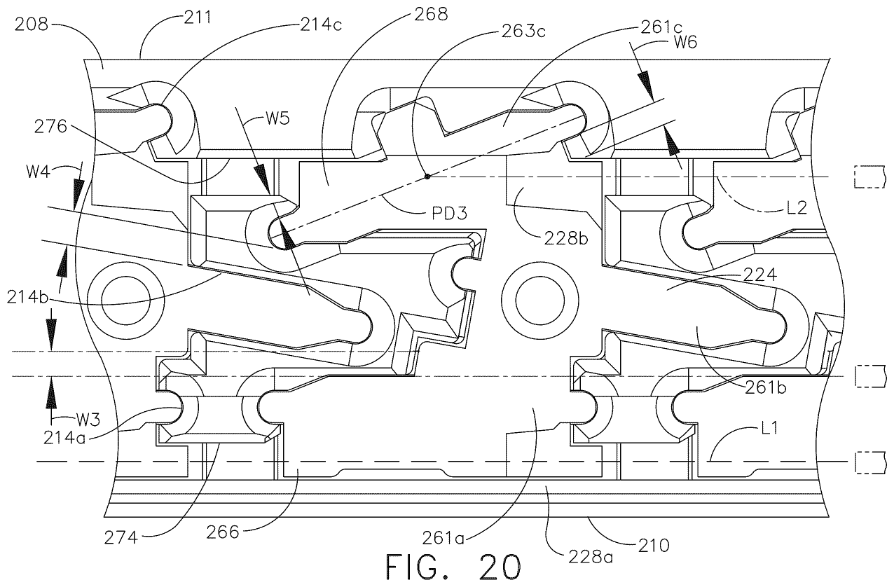

Referring primarily now to FIG. 20, the third proximal-to-distal axis PD3 for the outer row 216c intersects the longitudinal axis A along with the outer sled rail 236 translated at the geometric center 263c of the support column 261c in the outer cavity 214c. In other words, the outer rail 236 passes through the proximal-to-distal axis PD3 at the geometric center 263c and the channel slot 276 through the cartridge body 208 for receiving and guiding the outer rail 236 is positioned far enough inward away from the outside face 211 of the cartridge body 208 to maintain the minimum wall thickness between the outer cavity 214c and the outside face 211 and around the vertical portions (e.g. the staple legs) of the staple 220 in the outer cavity 214c. In such instances, the vertical portion of the staple (e.g. the staple legs) can be sufficiently supported during the firing motion.

Referring still primarily to FIG. 20, various dimensional relationships between walls in the cartridge body 208 are shown. Despite the compact arrangement of drivers and staples thereof, the cartridge body 208 can maintain a minimum wall thickness between the various cavities and channels therethrough. For examples, the cartridge body 208 can define minimum widths W3 (the minimum distance between an inner cavity 214a and an adjacent intermediate cavity 214b), W4 (the minimum distance between an intermediate cavity 214b and an adjacent outer cavity 214c), W5 (the minimum distance between the distal end 252 of the outer cavity 214c and the outer channel slot 276), and W6 (the minimum distance between the proximal end 250 of the outer cavity 214c and the outer rail channel). The minimum widths W3, W4, W5, and W6 can be at least 0.01 inches to ensure sufficient driver/staple support and/or injection molding flow paths. For example, the minimum widths W3, W4, W5, and W6 can be designed to be optimized at or around 0.01 inches by shifting the arrangement of drivers and staples to ensure the minimum width is maintained throughout the cartridge body 208. In other instances, at least one of the minimum widths W3, W4, W5, and W6 can be between 0.01 inches and 0.02 inches.

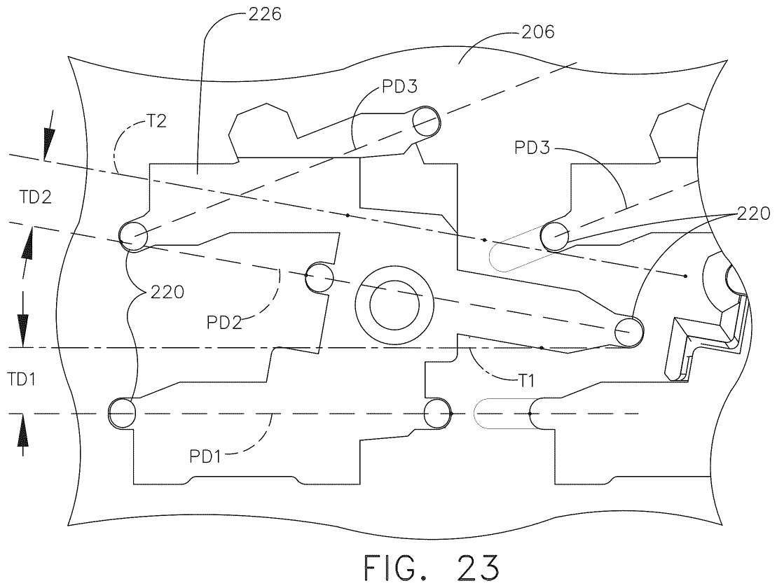

Referring primarily to FIG. 23, the compact array of nested drivers 224, 226 in the fastener cartridge assembly 206 provides a sufficiently tight staple line to seal the tissue while still allowing extendability and stretching of the stapled tissue. Exemplary distances are depicted in FIG. 23 in which illustrative tangent lines T1 and T2 are depicted. The tangent line T1 is the closest tangent line from a staple 220 in an intermediate cavity 214b to a staple 220 in an inner cavity 214a. The tangent line T2 is the closest tangent line from a staple 220 in an outer cavity 214c to a staple 220 in an intermediate cavity 214b. In various instances, the tangential distance TD from the tangent lines T1, T2 to the centerline of the adjacent row's tangency line can be the same row-to-row. For example, a first tangential distance TD1 from the tangent line T1 to the centerline of the staple 220 in the inner row 216a along the PD1 axis can be equal to, or substantially equal to, a second tangential distance TD2 from the tangent line T2 to the centerline of the staple 220 in the intermediate row 216b along the PD2 axis. In various aspects of the present disclosure, the tangential distances TD1 and TD2 can be less than 0.03 inches, and can be between 0.02 inches and 0.03 inches in certain instances. For example, the tangential distances TD1 and TD2 can be 0.21 inches. In other instances, the tangential distances TD1 and TD2 can be different.

Referring now to FIG. 24, the arrangement of nested drivers 224, 226 in the fastener cartridge assembly 206 provides differing parallel offsets between staples 220 in each row 216. For example, the staples 220 in the intermediate row 216b extend along PD2 axes, which are substantially parallel and offset by a first parallel offset distance PO1, and staples 220 in the outer row 216c extend along PD3 axes, which are substantially parallel and offset by a second parallel offset distance PO2. The second parallel offset distance PO2 is approximately double the first parallel offset distance PO1. For example, in instances in which the drivers 224, 226 are longitudinally spaced apart by a longitudinal length LL of 0.134 inches, the first parallel offset distance POI can be 0.023 inches, and the second parallel offset distance PO2 can be 0.049 inches. The reader will appreciate that if the longitudinal length LL is increased, the first parallel offset distance POI and the second parallel offset distance PO2 would also increase.

Referring again to FIG. 11, the intermediate cavities 214b are nested and positioned laterally between first cavities 214a and third cavities 214c. More specifically, an intermediate cavity 214b is positioned between proximally-positioned inner and outer cavities 214a, 214c and between distally-positioned inner and outer cavities 214a, 214c. The cavities 214 in the cartridge body 208 are positioned to form repeating patterns of cavity end clusters. For example, the cavities 214 form longitudinally-repeating high-density clusters 258 of cavity ends 250, 252 and longitudinally-repeating low-density clusters 256 of cavity ends 250, 252. The cavity ends 250, 252 correspond to staple legs when the staples 220 are installed in the cavities 214. Therefore, the arrangement of cavities corresponds to an arrangement of fasteners that are positioned to form longitudinally-repeating high-density clusters of fastener legs and longitudinally-repeating low-density clusters of fastener legs.

Referring still to FIG. 11, each high-density cluster 258 comprises three staple ends 250, 252, and each low-density cluster 256 comprises three staple ends. More specifically, each high-density cluster 258 includes an end 250 or 252 from an inner cavity 214a, an end 250 or 252 from an intermediate cavity 214b, and an end 250 or 252 from an outer cavity 214c. In such instances, each cluster of fastener legs comprises the leg of a fastener from an inner cavity 214a, an intermediate cavity 214b, and an outer cavity 214c. For example, the high-density clusters 258 include a distal end 252 of both an inner cavity 214a and an outer cavity 214c and a proximal end 250 of an intermediate cavity 214b. The low-density clusters 256 include a proximal end 250 of both an inner cavity 214a and an outer cavity 214c and a distal end 252 of an intermediate cavity 214b. Each cavity 214 includes one end 250 or 252 in a high-density cluster 258 and the opposite end 250 or 252 in a low-density cluster 256. Consequently, the high-density clusters 258 and the low-density clusters 256 form an alternating pattern along the longitudinal axis A.

The underside of the cartridge body 208 is depicted in FIG. 12, in which the sled 230 is in the proximal, unfired position at the proximal end 207 of the cartridge body 208. The sled can include upright rails that driving engage the underside of ramped surfaces on the drivers 224, 226 (FIG. 3) to drive the drivers 224, 226 toward the deck 212 (FIG. 3). The outer rails 236 are configured to engage outer ramps 228b (FIG. 6) and the inner rails 234 are configured to engage the inner ramps 228a (FIG. 6) of the drivers 224, 226. An inner rail 234 and an outer rail 236 on a first side of the longitudinal knife slot 210 are configured to simultaneously drive the inner ramp 228a and the outer ramp 228b, respectively, of a driver 224, 226. More specifically, the inner rail 234 and the outer rail 236 are longitudinally aligned, and the inner ramp 228a and the outer ramp 228b are longitudinally aligned. Moreover, the inner rail 234 and the outer rail 236 engage both ramps 228a and 228b at the same time and complete the upward pushing motion on the ramps 228a, 228b at the same time.

In various instances, the sled 230 and rails 234, 236 thereof are comprised of metal. For example, the sled 230 can be formed from (e.g. cast) from metal such that the rails 234, 236 can be narrower than conventional sled rails, which requires narrower channels through the cartridge body 208 and more material (i.e. thicker cavity walls) in the cartridge body 208. The narrower sled rails 234, 236 and passages therefor also accommodate the consolidated array of staple cavities 214 at the row-to-raw gradient of cavity angles/orientations relative to the longitudinal axis. For example, to accommodate the consolidated array of staple cavities 214, the outer rails 236 can be positioned to move along outer axes L2 that are laterally aligned with the cavities 214 and openings thereof, while the inner rails 234 are positioned to move along inner axes L1 that are positioned laterally inward from the inner cavities 214a. Referring primarily to FIG. 12, the outer cavities 214c define an outside outer row boundary and the inner cavities 214a define an inside inner row boundary. Each outer axes L2 can extend longitudinally between the outer outside row boundary and the inner inside row boundary.

In one aspect of the present disclosure, the inner axes L2 are positioned laterally inward from the inner cavities 214a between the inner row 216a and the longitudinal axis A on each side of the cartridge body 208. Moreover, the outer rails 236 can be configured to pass under the obliquely-oriented fasteners 220 of the outer cavities 214c in the outer rows 216c. For example, each fastener 220 in the obliquely-oriented outer cavities 214c can include an outside leg and an inside leg, and the outer axes L2 can be positioned between the outside leg and the inside leg of the fasteners 220 in the outer cavities 214c.

The compact arrangement of staple cavities 214 in the cartridge body 208 can optimize the small footprint of the fastener cartridge assembly 206 to fit three rows of staples at different angular orientations for each row. Moreover, the thickness of the cavity walls is limited by manufacturing processes (e.g. injection molded components require a minimum width for sufficient flow of the injected material in the mold). The cavity walls also must sufficiently support the drivers 224, 226 and staples 220 during a firing motion to ensure the drivers 224, 226 do not torque or become jammed in the cavities. Bowing or other deformation of the cavities walls can result in misfiring of the fastener cartridge assembly 206.

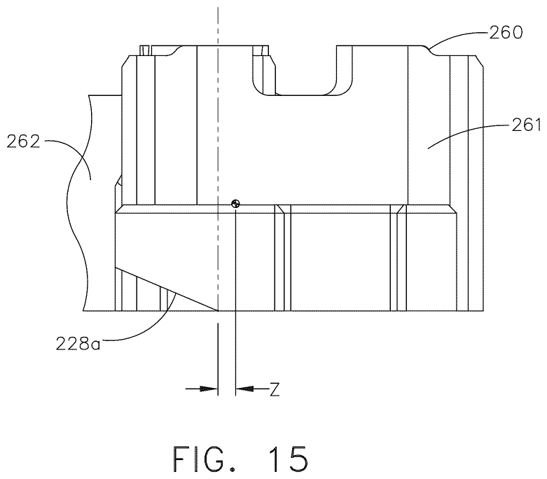

Referring still to FIG. 12 and also FIGS. 18 and 19, the inside wall of the cartridge body 208 defines a minimum width WI between the channel for the inner rail 234 of the sled 230 and the longitudinal knife slot 210. The outside wall of the cartridge body 208 defines a minimum width W2 between the proximal ends 250 of the outer cavities 214c and the outside face 211 of the cartridge body 208. The minimum widths W1 and W2 can be at least 0.01 inches to ensure sufficient driver/staple support and/or injection molding flow paths. In various instances, the minimum widths WI can be between 0.01 inches and 0.02 inches. For example, the minimum width W1 can be 0.012 inches, and the minimum width W2 can be 0.015 inches.

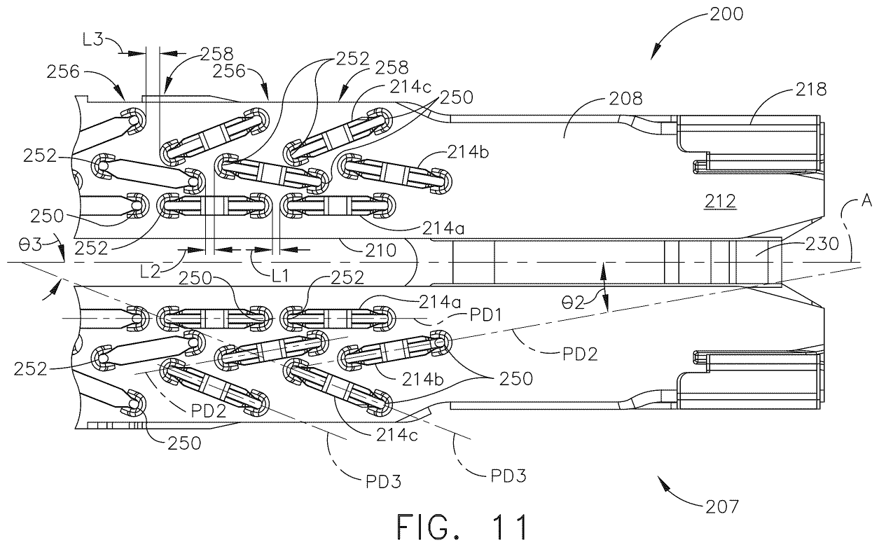

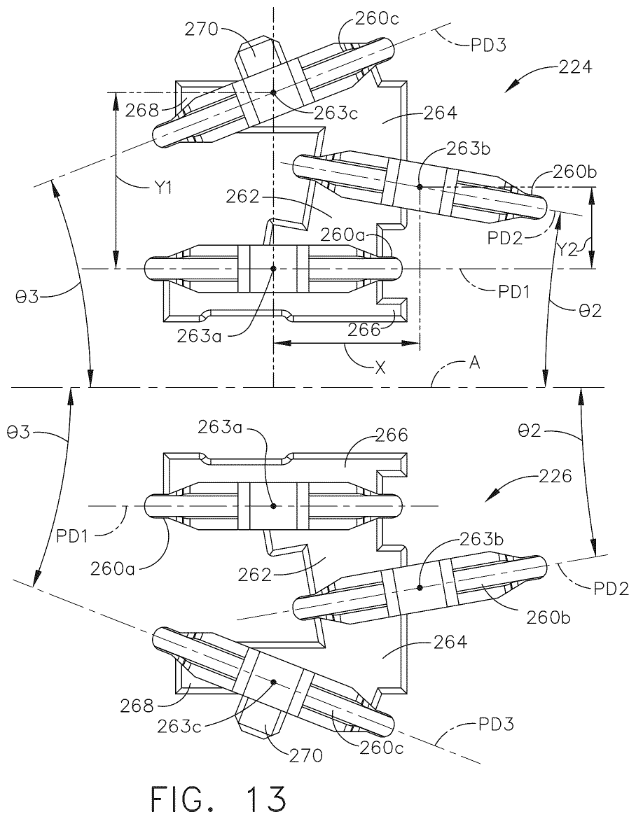

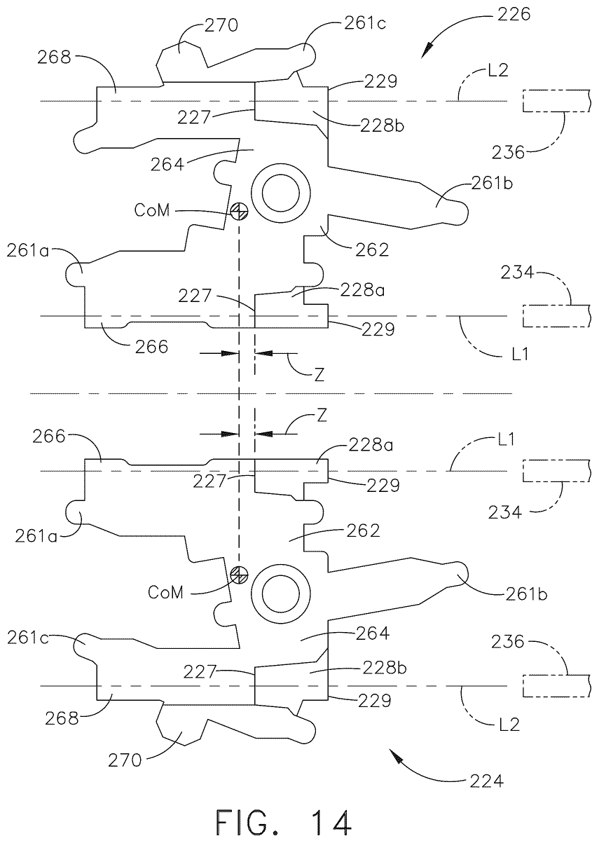

Referring primarily now to FIGS. 13-17, drivers 224, 226 are shown. The drivers 224, 226 include columns 261 supporting cradles 260. More specifically, the drivers 224, 226 are triple drivers, each having three columns 261a, 261b, and 261c and each column terminating in a cradle 260a, 260b, and 260c, respectively. The columns 261 are configured to move through the cavities 214 during a firing stroke. The sidewalls of the cavities 214 guide the columns 261 toward the cartridge deck 212 during the firing stroke to eject the staples 220 supported on the cradles 260a, 260b, and 260c. The cradles 260a, 260b, and 260c are configured to be overdriven with respect to the cartridge body 208 as further described herein. Each cradle 260a, 260b, and 260c defines a proximal-to-distal axis PD1, PD2, and PD3, respectively, that corresponds to the proximal-to-distal axis of the staple cavity in which it is positioned. More specifically, the inner cradles 260a are oriented along the first proximal-to-distal axes PD1, the intermediate cradles 260b are oriented along the second proximal-to-distal axes PD2, and the outer cradles 260c are oriented along the third proximal-to-distal axes PD3. PD1 is oriented at a first angle relative to the longitudinal axis A, PD2 is oriented at a second angle (θ2) relative to the longitudinal axis A, and PD3 is oriented at a third angle (θ3) relative to the longitudinal axis A.

In various instances, the first angle is zero and the PD1 axis is oriented parallel, or substantially parallel to the longitudinal axis A. Longitudinal alignment of an inner row of staples can help provide a robust seal along the cutline. The PD2 axis is angled away from the longitudinal axis A and cutline there along. Angling the intermediate cradle 260b, which is in the intermediate row and adjacent to the inner row, can increase the spacing between adjacent staple legs in the same row to improve pressure distribution, for example. Moreover, angling the intermediate cradle 260b (and staple thereon) away from the longitudinal axis A can facilitate tissue flow away from the cutline during the firing stroke. Tissue flow outward away from the cutline and innermost row can create a tighter staple line and improve hemostasis in certain instances. In certain instances, the second angle (θ2) is between 5 and 20 degrees, and can be 10 degrees, for example. The second angle (Θ2) is greater than the first angle, which is zero in the drivers 224, 226. PD3 is angled toward the longitudinal axis A and cutline there along. Angling the third cradle 260c, which is in the outermost row, can increase spacing between adjacent staple legs in the same row, which can improve pressure distribution in the stapled tissue, for example. Moreover, angling the third cradle 260c (and staple thereon) toward the longitudinal axis A can consolidate the staple line and allow nesting of adjacent proximal-to-distal drivers 224, 226 and staples thereon to minimize space between the staples and improve hemostasis. In certain instances, the third angle (θ3) is between 10 and 30 degrees, and can be 21.5 degrees, for example. The third angle (θ3) is greater than the first angle and the second angle (θ2) in the drivers 224, 226.

Referring primarily to FIG. 13, each cradle 260a, 260b, and 260c includes a geometric center 263a, 263b, and 263c, respectively, through which the proximal-to-distal axes (PD1, PD2, and PD3, respectively) pass through. On each driver 224, 226, the first geometric center 263a of the inner cradle 260a is longitudinally-aligned with the third geometric center 263c of the outer cradle 260b. Moreover, the third geometric center 263c is laterally offset from the first geometric center 263a by an inner-to-outer lateral offset Y1. Owing to the compact staple and driver arrangement, the inner-to-outer lateral offset Y1 can be minimized to less than 0.10 inches, and can be 0.075 inches, for example. In other instances, the inner-to-outer lateral offset Y1 can be between 0.07 inches and 0.085 inches, for example. Furthermore, on each driver 224, 226, the second geometric center 263b of the intermediate cradle 260b is longitudinally offset from the first geometric center 263a and the third geometric center 263c by a longitudinal stagger X. Moreover, the second geometric center 263b is laterally offset from the first geometric center 263a by an inner-to-intermediate lateral offset Y2. Owing to the compact staple and driver arrangement, the longitudinal stagger X can be minimized to less than 0.080 inches, and can be 0.062 inches, for example. In other instances, the longitudinal stagger X can be between 0.07 inches and 0.06 inches, for example. Additionally, the inner-to-intermediate lateral offset Y2 can be minimized to less than 0.050 inches, and can be 0.035 inches, for example. In other instances, the inner-to-intermediate lateral offset Y2 can be between 0.045 inches and 0.03 inches, for example.

The drivers 224, 226 include an inner bridge 262 extending between the inner column 261a and the intermediate column 261b and also include an outer bridge 264 extending between the intermediate column 261b and the outer column 261c. The bridges 262, 264 connect the columns 261 to form a triple driver spanning three rows of cavities. The drivers 224, 226 also include an inside flange 266 extending laterally inward from the inner column 261a and an outside flange 268 extending distally and laterally outward from the outside column 261c. The inside and outside flanges 266, 268 movably engage the wedge-shaped inner and outer rails 234, 236, respectively, during the firing stroke. For example, the underside of the inside flange 266 includes the inner ramp 228a, and the underside outside flange 268 includes the outer ramp 228b. The rails 234, 236 are configured to move along the undersides of the flanges 266, 268 and/or along the ramps 228a, 228b, respectively, during a firing motion. Referring primarily to FIG. 14, as further described herein, the inner rails 234 (FIG. 11) are configured to move along first longitudinal axes L1, and the inner flange 266 extends laterally inward from the inner column 261a to position the inner ramp 228a in alignment with the first longitudinal axes L1. Moreover, the outer rails 236 (FIG. 11) are configured to move along second longitudinal axes L2, and the outer flange 268 extends laterally outward from the outer column 261c to extend the engagement surface between the outer rails 236 and the respective driver 224, 226 distal to the outer ramp 228b.

The drivers 224, 226 further include at least one upright rib 270, which is configured to be received in a recess in the cartridge body 208. Engagement between the rib 270 and the recess further guides the drivers 224, 226 during the firing motion and prevents rotation and jamming of the drivers 224, 226 in the cavities 214.

Referring primarily to FIG. 14, the drivers 224, 226 further include a center of mass (“COM”) between the inner column 261a and the intermediate column 261b. The ramps 228a, 228b each include a leading distal edge 229 and a trailing proximal edge 227. The CoM of the drivers 224, 226 is positioned proximal to the trailing proximal edges 227 thereof. More specifically, the COM is longitudinally offset from the trailing proximal edges 227 in the proximal direction by a distance Z in FIG. 14. In certain instances, the distance Z can be less than 0.01 inches. In certain instances, the distance Z can be between 0.004 inches and 0.008 inches. For example, the distance Z is 0.0057 inches in various aspects of the present disclosure. The three-dimensional position of the COM is adjusted by adjusting the geometry of the inner bridge 262 and outer bridge 264. For example, by extending the inner bridge 262 distally, the CoM of the drivers 224, 226 shifts distally. In such instances, the length of the inner bridge 262, for example, can by adjusted to ensure the CoM is distally beyond the trailing proximal edges 227, which can prevent driver roll during a firing motion in certain instances.

Referring now to FIGS. 18 and 19, portions of the cartridge body 208 are shown with drivers 224, 226 removably positioned in the cavities 214 thereof. The cartridge body 208 includes an outside wall supporting the outside edge of the drivers 224, 226, and an inside wall supporting the inside edge of the drivers 224, 226 adjacent to the longitudinal knife slot 210. The inside contoured walls of the cartridge body 208 between the outside wall and the inside wall form an island 277 defining the cavities 214, upright cavities and tracks for receiving (and guiding) the drivers 224, 226 and staples 220, and channel slots 274, 276 (FIG. 18) for receiving (and guiding) the rails 234, 236 of the sled 230 during the firing stroke. Owing to the compressed arrangement of staple cavities 214 in the cartridge body 208, the island 277 can be separated from the outside wall and the inside wall by narrow channel slots 274, 276 along which the narrow rails 234, 236, respectively, of the sled 230 are configured to pass during the firing stroke.

The island 277 defines stand up features in the cartridge body 208, which are supported by opposing ribs 278 extending down from the deck 212 and forming a thicker portion of the deck 212 than the adjacent portions of the deck 212. The increased thickness provided by the opposing ribs 278 is further shown in FIG. 10, for example. The opposing ribs 278 can be dimensioned to substantially fill the longitudinal space between consecutive drivers 224, 226 and the vertical space from the deck 212 to the longitudinal channel for the rails 234, 236. The clearance between the top of the sled 230 and the opposing ribs 278 can be minimized to sufficiently support the island 277 and prevent bending or twisting of the islands 277 during the firing stroke. For example, significant firing forces during a firing stroke can exert a torque on the islands 277. Without sufficient support, the islands 277 are prone to bending forward in certain instances. The opposing ribs 278 support the islands 277 and provide a flow path for plastic resin, for example, to flow into each island 277. The deck 212 alone is too thin in various instances to permit sufficient flow into the islands during the molding process.

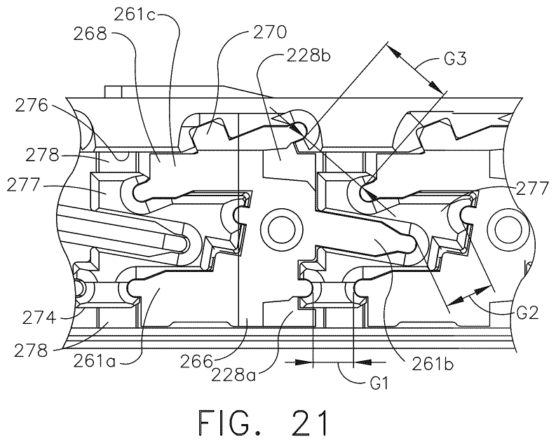

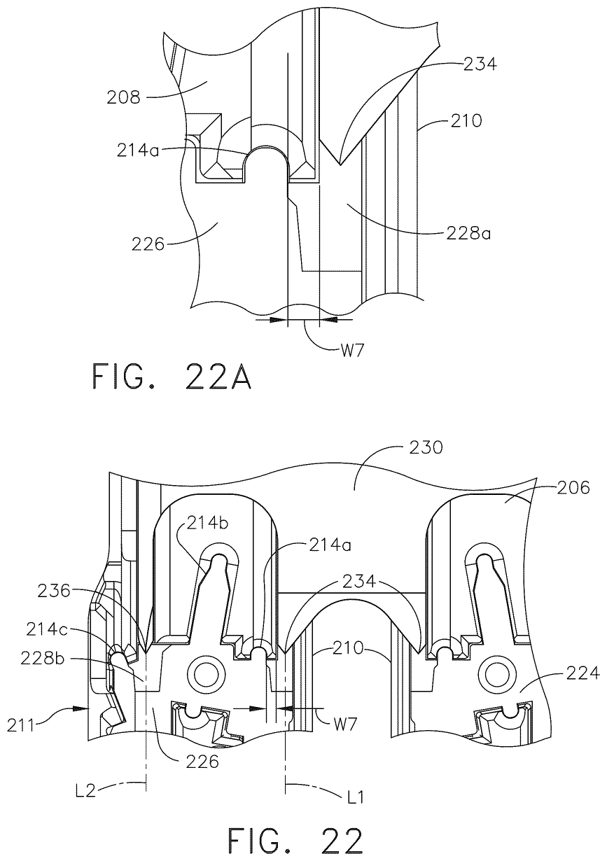

Referring primarily now to FIGS. 22 and 22A, a portion of the fastener cartridge assembly 206 is shown. The inner rails 234 of the sled 230 are configured to move along the first longitudinal axis L1, and the outer rails 236 of the sled 230 are configured to move along the second longitudinal axis L2. The second longitudinal axis L2 defines a channel slot 276 (FIG. 18) in the cartridge body 208 through which the outer rail 236 translates during the firing stroke. The outer support column 261c of the driver 226 extends into the channel slot 276, and is lifted out of the channel slot 276 when the outer rail 236 engages the outer ramp 228b on the underside of the driver 226 and along the underside of outer support column 261c and outer flange, or tail, 268. The inner longitudinal axis L1 defines a channel 274 (FIG. 18) in the cartridge body 208 through which the inner rail 234 translates during a firing stroke. The rails 234, 236 can be comprised of metal to be sufficiently robust even when reduced to a narrowed width to fit in the narrowed channel slots 274, 276. The outside surface of the inner rail 234 is positioned a minimum width W7 from the inner edge of the inner cavity 214a where the cartridge body 208 forms the track at the end of the inner cavity 214a for supporting the vertical portion (e.g. the staple legs) of the staple 220 during a firing stroke. In various instances, the minimum width W7 can be approximately 0.010 inches or less than 0.010 inches. For example, the minimum width W7 can be 0.007 inches, which can be the limit for plastic injection molded components. By reducing the minimum width W7 to the minimum manufacturable dimension, the width of the fastener cartridge assembly 2006 can be minimized to maintain a small footprint for minimally-invasive surgical procedures, for example. At the minimum width W7, the cartridge body 208 can have enough material, i.e. wall thickness, to hold the staple 220 upright in the inner cavity 214a as the staple 220 is fired from the inner cavity 214a and into tissue.

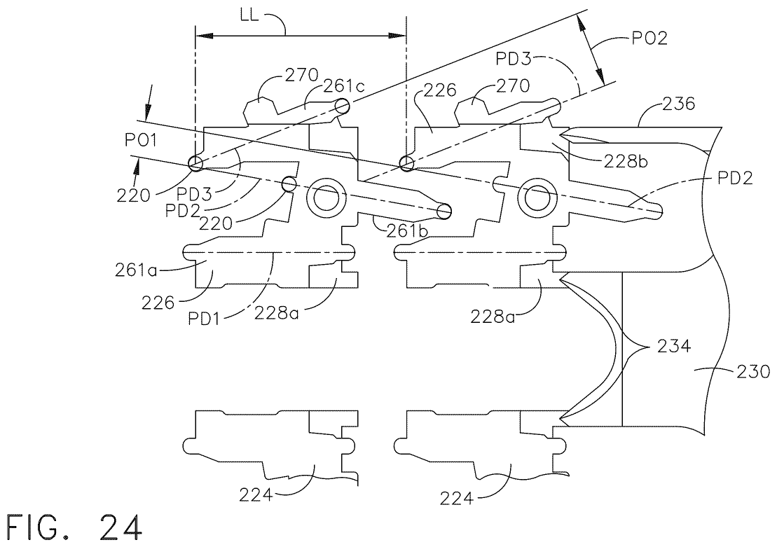

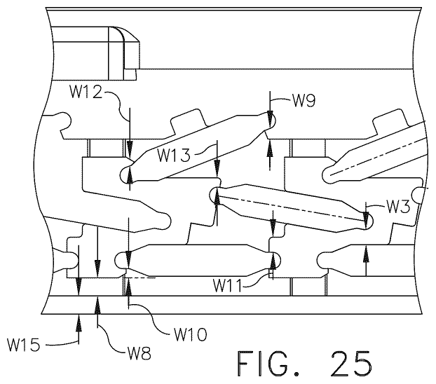

Minimum widths W1 and W2 (FIGS. 12); W3, W4, W5 and W6 (FIGS. 20); and W7 (FIG. 22) are further described herein, as well as the angular orientation of the proximal-to-distal axes PD1, PD2, and PD3 (e.g. angles θ2 and θ3) (FIGS. 11 and 13); longitudinal lengths L1, L2, and L3 (FIG. 11); longitudinal stagger X (FIG. 13); lateral offsets Y1 and Y2 (FIG. 13); and staple gaps G1, G2, and G3 (FIG. 21); tangential distances TD1 and TD2 (FIG. 23); and parallel offset distances PO1 and PO2 (FIG. 24); for example. Certain exemplary dimensional relationships for the fastener cartridge assembly 206 are also shown in FIG. 25. For example, the minimum wall thickness for plastic flow in injection molded components is minimum width W3, which is 0.010 inches, in various aspects of the present disclosure.

Referring still to FIG. 25, the minimum width W15 of the cartridge wall between the longitudinal knife slot 210 and the inner rail 234 (FIG. 3) of the sled 230 can be limited by manufacturing limitations for injection molded components, for example. The minimum width W15 can be less than 0.020 inches, and is 0.012 inches in various aspects of the present disclosure. In other instances, the minimum width W15 can be 0.01 inches, for example. The minimum width W8 of the channel slot 274 for the inner rail 234 can also be reduced to fit a narrower metal rail, such as the inner rail 234. For example, the minimum width W8 of the channel slot 274 (and channel slot 276) can be less than 0.02 inches, and is 0.012 inches in various aspects of the present disclosure. In other instances (where the sled rails are less than 0.01 inches), the minimum width W15 can be 0.01 inches.

FIG. 25 further depicts edge dimensions for capturing the vertical portion (e.g. the staple legs) of the staple 220 during a firing motion. For example, edge widths W9, W10, W11, W12, and W13 are configured to support the staple legs during firing motions thereof. In various instances, the staples 220 can be V-shaped staples, for example, with outward splaying legs, as further described herein. The proximal and/or distal edges of the cartridge body around the staple cavities are configured to engage the staple legs and support the staple legs as the drivers 224, 226 lift the staple bases toward the cartridge deck. Though not a complete wall in the cartridge body 208 (which are subject to different size limitations further described herein), these edges or extensions around the cavities 214 define a key limitation on the compact array of cavities 214, drivers 224, 226, and staples 220 thereon. Manufacturing limitations and material strength limitations, for example, can compel a minimum edge width, which can be less than 0.010 inches, for example. The edge widths W9, W10, W11, W12, and W13 are approximately 0.006 inches in various aspects of the present disclosure. In certain instances, referring to edge widths W9, W12, W13 (around the obliquely-oriented cavities 214, support columns 261, and staples 220 in the intermediate row 216b and outer row 216c) the edge widths can be on an angle and the minimum edge width along the angled cavity where the vertical portions of the staple are supported is compelled by the manufacturing and material limitations of the cartridge body 208.

In various instances, a compact arrangement of staple cavities and staples therein can be effected by the geometry and arrangement of staple forming pockets in the anvil. For example, the forming pockets can include a pair of cups, and each cup can be configured to receive one staple leg. The cups are configured to catch the tips of the staple legs and deform the staple legs along a predefined trajectory toward their formed configurations. In order to catch the tips of the staple legs, which are subjected to tissue flow and associated forces during a firing stroke, each forming pocket can be larger than the target contact area for the tips of the staple legs. The staple forming pockets and cups thereof can take up a larger footprint on the anvil than the footprint of the corresponding staple cavity in the fastener cartridge assembly. Consequently, the limitations on staple cavity arrangements described herein can be further limited by the nestability of the forming pockets proximal-to-distal and row-to-row.

In various instances, the available space for forming pockets on the forming surface of an anvil is further reduced proximal to a tissue stop, which can take up real estate along the outside edge of the forming surface. The placement of forming pockets proximal to a tissue stop can improve sealing and/hemostasis by further extending the stapling line for the associated transection. For the fastener cartridge assembly 200, for example, the arrangement of forming pockets can extend proximally beyond a tissue stop plane defined by the tissue stops, which enables proximal extension of the staple line.

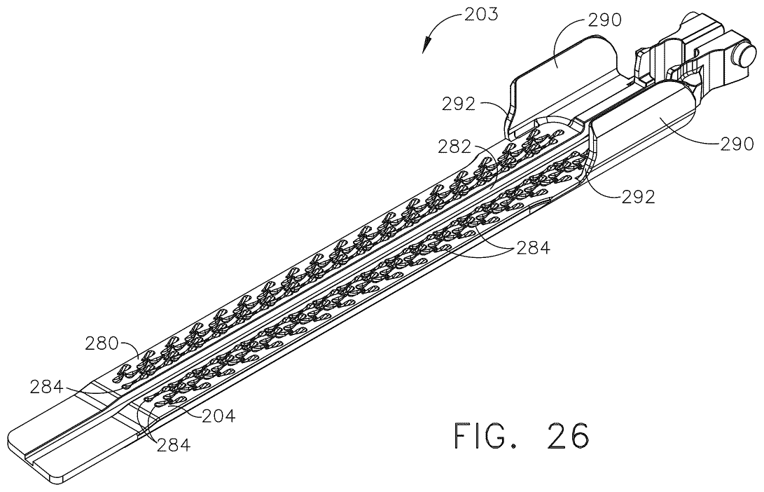

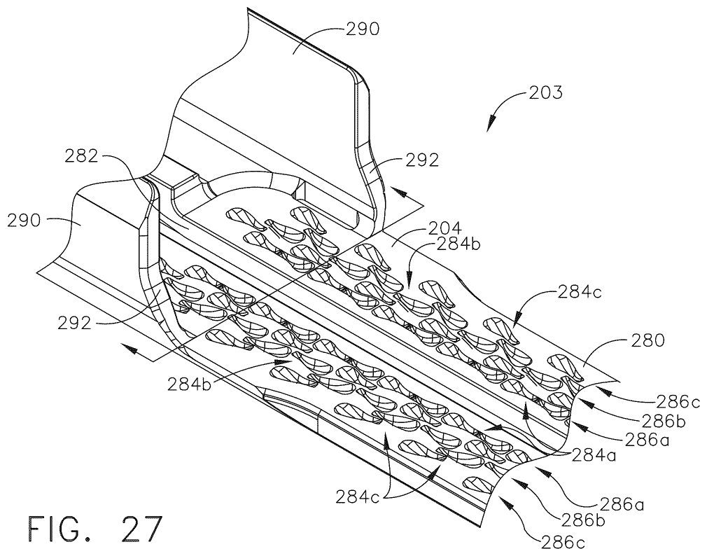

Referring primarily to FIGS. 26-28, the anvil 204 includes an anvil forming surface 280 with a longitudinal slot 282 and forming pockets 284 defined therein. The forming pockets 284 are arranged in multiple rows 286 on either side of the longitudinal slot 282. The forming pockets 284 are configured to receive the legs of the fasteners and the guide the fastener legs along a predefined curvature into a formed configuration.

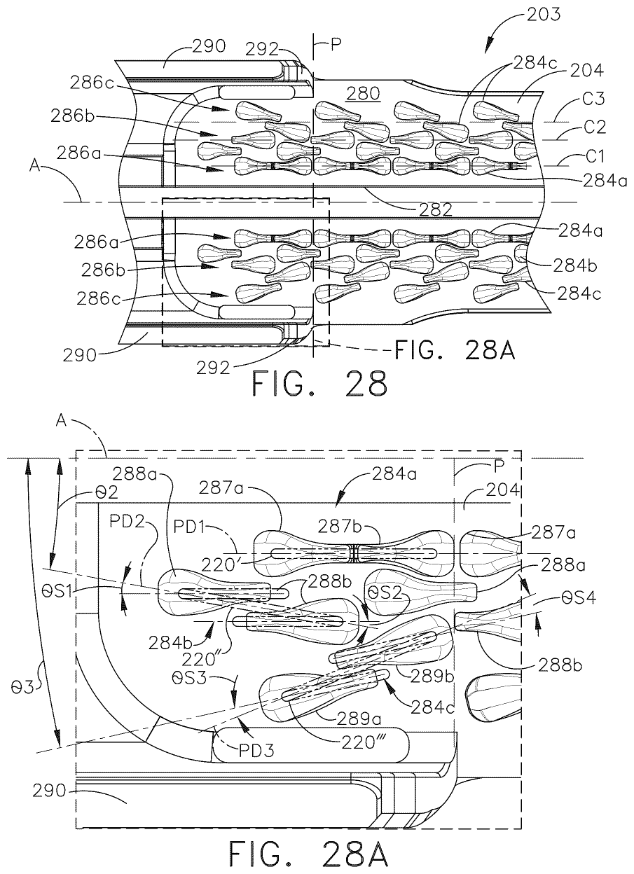

Referring primarily to FIG. 28, the cartridge body 208 includes three rows 286 on both sides of the longitudinal slot 282. The rows includes an inner row 286a comprised of inner forming pockets 284a, an intermediate row 286b comprised of intermediate forming pockets 284b, and an outer row 286c comprised of outer forming pockets 284c.

In other instances, the forming surface 280 can include additional rows of forming pockets or fewer rows of forming pockets on one or both sides of the longitudinal slot 282. In certain instances, the forming surface 280 can be symmetrical about a longitudinal axis A and the rows of forming pockets can be symmetrical about the longitudinal axis A. In other instances, the rows of forming pockets can be asymmetrical about the longitudinal axis A.

The inner forming pockets 284a in the anvil 204 are longitudinally-aligned with the longitudinal axis A, i.e. extend along a first proximal-to-distal axis PD1, or pocket centerline, that is parallel, or substantially parallel, to the longitudinal axis A. Each inner forming pocket 284a comprises individual cups 287a, 287b. The intermediate forming pockets 284b and the outer forming pockets 284c are obliquely-oriented relative to the longitudinal axis A. The intermediate forming pockets 284b each define a second proximal-to-distal axis PD2, or pocket centerline, from the proximal end to the distal end thereof, and the second proximal-to-distal axes PD2 are angled away from the longitudinal axis A. Though the PD2 axes of each intermediate forming pocket 284b (defined by both cups 288a, 288b of the intermediate forming pockets 284b) is angled away from the longitudinal axis, the individual cups 288a, 288b forming the intermediate forming pockets 284b are oriented parallel to the longitudinal axis A and are laterally offset from each other, which results in the oblique orientation of the PD2 axes.

The outer forming pockets 284c each define a third proximal-to-distal axis PD3, or pocket centerline, from the proximal end to the distal end thereof, and the PD3 axes are angled toward the longitudinal axis A. Each cup 289a, 289b of the outer forming pockets 284c are also oriented at an oblique angle with respect to the longitudinal axis A. The longitudinal alignment of the inner forming pockets 284a and the angled orientations of the intermediate forming pockets 284b and the outer forming pockets 284c allow the intermediate forming pockets 284b to nest closely between the inner forming pockets 284a and the outer forming pockets 284c. The nested arrangement of forming pockets positioned at different angular orientations can provide a compact arrangement of forming pockets and a correspondingly tight staple line, while still allowing sufficient stretch to accommodate movement of the stapled tissue. Various exemplary dimensions are further described herein, which enable the array of drivers, staples, forming-pockets, and so on to fit within the small form factor of a fastener cartridge assembly for minimally-invasive surgeries such as minimally-invasive thoracic surgery, for example.

The second proximal-to-distal axes PD2 are oriented at a second pocket angle θ2 relative to the longitudinal axis A, and the third proximal-to-distal axes PD3 are oriented at a third pocket angle θ3 relative to the longitudinal axis A. The third pocket angle θ3 is greater than the second pocket angle θ2. For example, the second pocket angle θ2 can be between 8 degrees and 12 degrees (e.g. approximately 10 degrees), and the third pocket angle θ3 can be between 18 degrees and 25 degrees (e.g. approximately 21.5 degrees). In various instances, the angle of the proximal-to-distal axes PD1, PD2, PD3 of the forming pockets can match or be equal to the angle of the proximal-to-distal axes PD1, PD2, PD3 of the staple cavities 214 (and staples 220 therein). Both the second and third pocket angles θ2, θ3 are greater than the angle at which the first forming pockets 284a are oriented relative to the longitudinal axis. For example, where the first forming pockets 284a are parallel to the longitudinal axis A, the angle of the first forming pockets 284a relative to the longitudinal axis A can be zero. In various instances, the angular orientation of proximal-to-distal axes defined between ends of the forming pockets 284 can define an increasing gradient from the longitudinal centerline outbound toward the lateral sides of the anvil 204.

Referring primarily to FIG. 28A, each forming pocket includes a pair of forming cups 287a, 287b, 288a, 288b, 289a, 289b configured to receive the staple legs. More specifically, the inner forming pockets 284a include an inner proximal cup 287a and an inner distal cup 287b, which are aligned and connected. Moreover, the inner cups 287a, 287b are aligned with a central inner axis C1 (FIG. 28), which is collinear with the PD1 axes. The inner proximal cup 287a is configured to receive a proximal staple leg and the inner distal cup 287b is configured to receive a distal staple leg of the same staple. The staple legs can be directed along the entry ramp of each inner cup 287a, 287b toward the first proximal-to-distal axis PD1 thereof, can bend at the apex of each inner cup 287a, 287b, and be further deformed by an exit ramp of the cup 287a, 287b back toward the fastener cartridge assembly 206 (FIG. 1) and along the proximal-to-distal axis PD1 thereof. The inner forming pockets 284a are structured to deform the staples into a planar, or substantially planar, configuration, in which the staple legs and base are aligned with a plane through the first proximal-to-distal axis PD1 thereof.

The intermediate forming pockets 284b include an intermediate proximal cup 288a and an intermediate distal cup 288b, which are independent. The intermediate cups 288a, 288b are each oriented longitudinally, i.e. parallel to the longitudinal axis A and to each other; and are laterally offset and disconnected from each other. Moreover, the intermediate cups 288a, 288b are equilaterally-spaced apart from a central intermediate axis C2 (FIG. 28) that defines the intermediate row 286b and is oriented parallel to the longitudinal axis A. The intermediate proximal cup 288a is configured to receive a proximal staple leg and the intermediate distal cup 288b is configured to receive a distal staple leg. The staple legs can be directed along the entry ramp of each intermediate cup 288a, 288b, can bend at the apex of each intermediate cup 288a, 288b, and be further deformed by an exit ramp of the cup 288a, 288b to back toward the fastener cartridge assembly 206 (FIG. 1) but diverging away from the second proximal-to-distal axis PD2 thereof. The intermediate forming pockets 284b are structured to deform the staples 220 into a non-planar configuration, in which the staple legs and base are deformed out of alignment with each other and out of alignment with a plane through the second proximal-to-distal axes PD2 thereof.

The outer forming pockets 284c include an outer proximal cup 289a and an outer distal cup 289b, which are disconnected. The outer cups 289a, 289b are each obliquely-oriented relative to the longitudinal axis A and parallel to each other, but offset and independent of each other and from a central outer axis C3 (FIG. 28) that defines the outer row 286c. The outer proximal cup 289a is configured to receive a proximal staple leg and the outer distal cup 289b is configured to receive a distal staple leg. The staple legs can be directed along the entry ramp of each outer cup 289a, 289b, can bend at the apex of each outer cup 289a, 289b, and be further deformed by an exit ramp of the cups 289a, 289b back toward the fastener cartridge assembly 206 (FIG. 1) but diverging from the third proximal-to-distal axis PD3 thereof. The outer forming pockets 284c are structured to deform the staples into a non-planar configuration, in which the staple legs and base are deformed out of alignment with each other and out of alignment with a plane through the third proximal-to-distal axes PD3 thereof.

Planar and non-planar staples and anvils for forming the same are further described in U.S. patent application Ser. No. 17/211,145, titled METHOD OF USING A POWERED STAPLING DEVICE, filed Mar. 24, 2021, which is incorporated by reference herein in its entirety.

The inner cups 287a, 287b of the inner forming pocket 284a are connected; however, the intermediate cups 288a, 288b of the intermediate forming pocket 284b and the outer cups 289a, 289b of the outer forming pockets 284 are discrete and independent cups.

In various instances, the distance between the central inner axis C1 and the central intermediate axis C2 can be less than the distance between the central intermediate axis C2 and the central outer axis C3. For example, the distance between the central inner axis C1 and the central intermediate axis C2 can be less than the width of the forming pockets. Additionally or alternatively, the distance between the central intermediate axis C2 and the central outer axis C3 can be greater than the width of the forming pockets 284.

In various aspects of the present disclosure, the portion of the forming surface 280 filled with forming pockets 284 is maximized within the constraints imposed by how close the forming pockets can be made during a manufacturing process. In certain instances, 35% to 55% of the total tissue-contacting surface of the forming surface 280 can be within the boundaries of a forming pocket. For example, the total area of the forming surface 280 within the boundaries of forming pockets 284 divided by the total area of the forming surface 280 is 37.4% in the anvil 204 in FIG. 26. In the foregoing example, the total area of the forming surface can be 0.782 square inches, and the total area of the forming surface 280 within the boundaries of the forming pockets 284 can be 0.293 square inches.

The anvil 204 further comprises tissue stops 290 projecting away from the forming surface 280. Each tissue stop 290 can include an upright distal-facing edge 292. The distal-facing edges 292 define a tissue stop plane P, which blocks tissue from protruding proximally beyond the pivot point between the jaws 201, 203 (FIG. 1). If tissue was positioned past the tissue stop plane P, the surgical stapling assembly 100 may be unable to sufficiently close and clamp onto tissue, for example. Referring primarily to FIGS. 28 and 28A, one whole inner forming pocket 284a, one whole intermediate forming pocket 284b, and one whole outer forming pocket 284c are positioned proximal to the tissue stop plane P. Moreover, in such instances, at least one longitudinally-oriented staple pocket 284a and two obliquely-oriented staple pockets 284b, 284c are positioned proximal to the tissue stop plane P. In various aspects of the present disclosure, at least one forming pocket configured to form planar staples (e.g. inner forming pocket 284a) and at least one forming pocket configured to form non-planar staples (e.g. intermediate forming pocket 284b and outer forming pocket 284c) are positioned proximal to the tissue stop plane P.

In various aspects of the present disclosure, a staple 220 fired from an inner row 214a can be formed proximal to the tissue stop plane P, a staple 220 fired from an intermediate row 214b can be formed proximal to the tissue stop plane P, and a staple 220 fired from an outer row 214c can be formed proximal to the tissue stop plane P. Moreover, in such instances, at least one longitudinally-aligned staple 220 (formed by a first forming pocket 284a) and at least one obliquely-oriented staple 220 (from by a second forming pocket 284b or a third forming pocket 284c) in a staple line can be proximal to the tissue stop plane P. In various aspects of the present disclosure, at least one planar formed staple 220 (formed by a first forming pocket 284a) and at least one non-planar formed staple 220 (from by a second forming pocket 284b or a third forming pocket 284c) in a staple line can be proximal to the tissue stop plane P.

Second and third forming pockets 284b and 284c are three-dimensional forming pockets. The structure of the three-dimensional forming pockets can be adjusted to fit the anvil geometry constraints and/or to maximum staple leg crossover. With respect to non-planar staples, staple leg crossover can improve homeostasis in certain instances. The three-dimensional forming pockets (e.g. 284b and 284c) are dimensioned with varying splay angles between the staple base, or crown, and the staple legs, and with varying pocket radii to fit in the footprint limitation of the anvil 204 while maximizing staple leg cross over.

Referring primarily to FIG. 28A, a first splay angle θS1 is defined between the PD2 axis and an axis defined by the proximal leg of the formed staple 220″, and a second splay angle θS2 is defined between the PD2 axis and an axis defined by the distal leg of the formed staple 220″. The first splay angle θS1 and the second splay angle θS2 are the same in FIG. 28A. For example, both splay angles θS1 and θS2 can be nine degrees. The splay angles θS1 and θS2 can be between five and fifteen degrees, in various aspects of the present disclosure. In certain instances, the first splay angle θS1 and the second splay angle θS2 can be different.

A third splay angle θS3 is defined between the PD3 axis and an axis defined by the proximal leg of the staple 220′″, and a fourth splay angle θS4 is defined between the PD3 axis and an axis defined by the distal leg of the staple 220′″. The third splay angle θS3 and the fourth splay angle θS4 are the same in FIG. 28A. For example, both splay angles θS3 and θS4 can be eight degrees. The splay angles θS3 and θS4 can be between five and fifteen degrees, in various aspects of the present disclosure. In certain instances, the third splay angle θS3 and the fourth splay angle θS4 can be different.

The intermediate forming pockets 284b can also have pocket radii of 0.055 inches and the outer forming pockets 284c can have a pocket radii of 0.060 inches, for example. In other instances, the splay angles and/or pocket radii of non-planar staples formed in different rows can be the same.

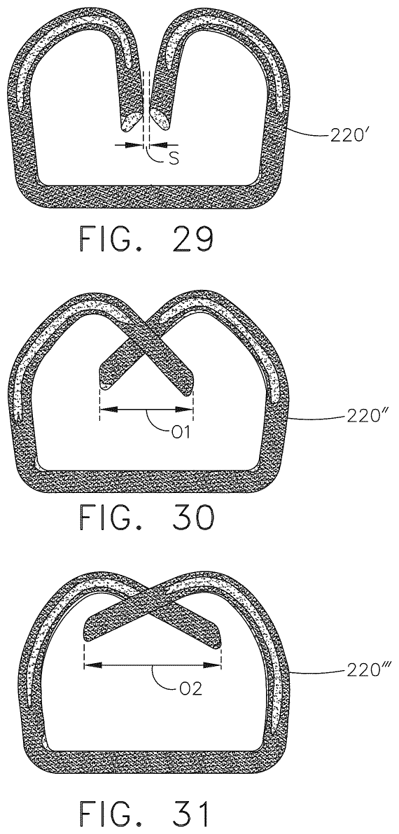

The staple leg crossover can also vary between non-planar staples in the same staple line. For example, in FIG. 28A, the formed staple 220″ (formed by the intermediate forming pocket 284b) can have a first overlap O1 (FIG. 30) between the tips of the crossed staple legs, and the formed staple 220′″ (formed by the outer forming pocket 284c) can have a second overlap O2 (FIG. 31) between the tips of the crossed staple legs. The formed staple 220′ (formed by the inner forming pocket 284a) can define a space S (FIG. 29) between the tips of the staple legs rather than an overlap. FIGS. 29-31 depicts the different formed staple geometries for formed staples 220′, 220″, and 220′″ formed by different forming pockets 284. Notably, the unformed staples 220 can be identical in various aspects of the present disclosure. The formed height of the staples 220′ (FIG. 29), 220″ (FIGS. 30), and 220′″ (FIG. 31) can be the same height. Nonetheless, the staples have assumed different formed geometries-the formed staple 220′ forming a planar staple with a space S between the tips of the staple legs, the formed staple 220″ forming a non-planar staple with an overlap O1 between the tips of the staple legs, and the formed staple 220′″ forming a non-planar staple with an overlap O2, which is different than the overlap O1, between the tips of the staple legs. Referring to FIGS. 30 and 31, the first overlap O1 is 0.015 inches and the second overlap O2 is 0.023 inches, for example.

In other instances, the formed heights of the staples 220′, 220″, and 220′″ can vary row-to-row. Additionally or alternatively, the unformed staples can vary row-to-row. For example, to better fit within a compact footprint, the staples can have different crown lengths in the different rows on the same side of the cartridge body 208 and/or different leg lengths.

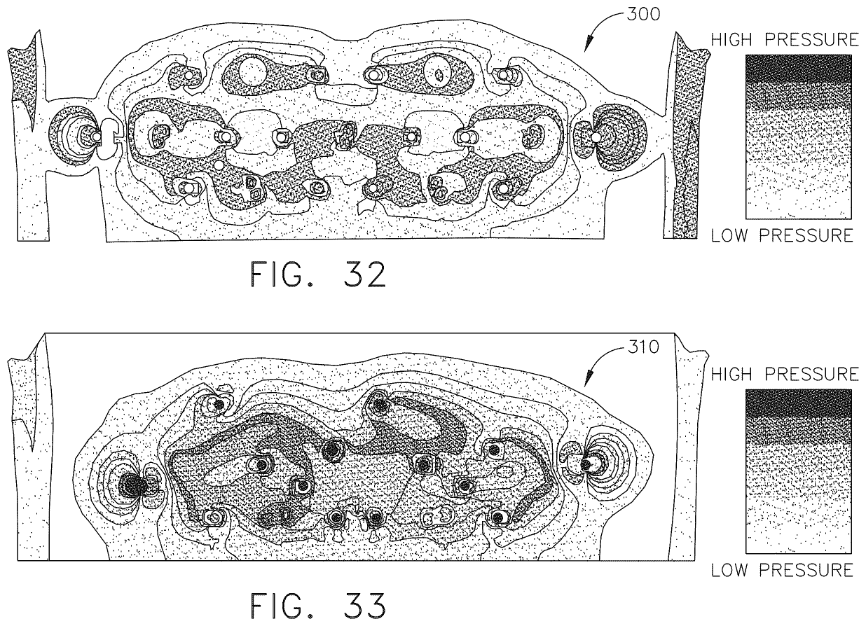

Example pressure distributions 300, 310 in different staple lines are shown in FIGS. 32 and 33. The pressure distribution 300 in FIG. 32 results from three adjacent rows of longitudinally-aligned, planar staples, and the pressure distribution 310 results from the combination of longitudinally-aligned, planar staples with obliquely-oriented, non-planar staples formed with the surgical stapling assembly 100 (FIG. 1), for example. By optimizing the forming pocket geometries, angular orientations of the forming pockets and staples, widths of the staple crowns and splay of the non-planar staple legs with the geometric constraints of the anvil 204 and entire surgical stapling assembly 100, a wider pressure distribution can be achieved. More specifically, a wider band of desirable pressure is achieved spanning between the inner and outer staple rows in the pressure distribution 310 compared to the speckled array of higher pressure and lower pressure regions in the pressure distribution 300.

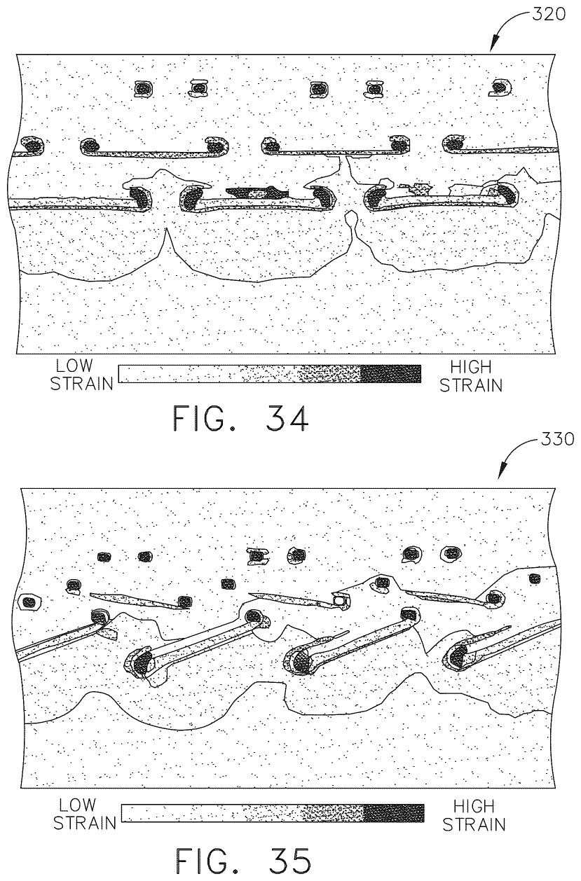

Example strain contour plots 320, 330 for different staple lines are shown in FIGS. 34 and 35. The strain contour plot 320 in FIG. 34 results from three adjacent rows of longitudinally-aligned, planar staples, and the pressure distribution 330 results from the combination of longitudinally-aligned, planar staples with obliquely-oriented, non-planar staples formed with the surgical stapling assembly 100 (FIG. 1), for example. While optimizing the staple line for an improved compressive profile, as shown in FIG. 33, for example, obliquely-oriented staples can also improve staple line stretch, flexibility, or compliance. A more compliant staple line can be less likely to result in an air leak at puncture sites, such as in the lung during a thoracic surgical procedure, for example. More specifically, the global stretch along the staple line in plot 320 can be greater than the global stretch along the staple line in plot 310. Under pressure and stretch loading conditions during a test, the staple line of plot 320 achieved global stretch along the staple line of 7.08% and the staple line of plot 330 achieved global stretch along the staple line of 8.03%, for example.

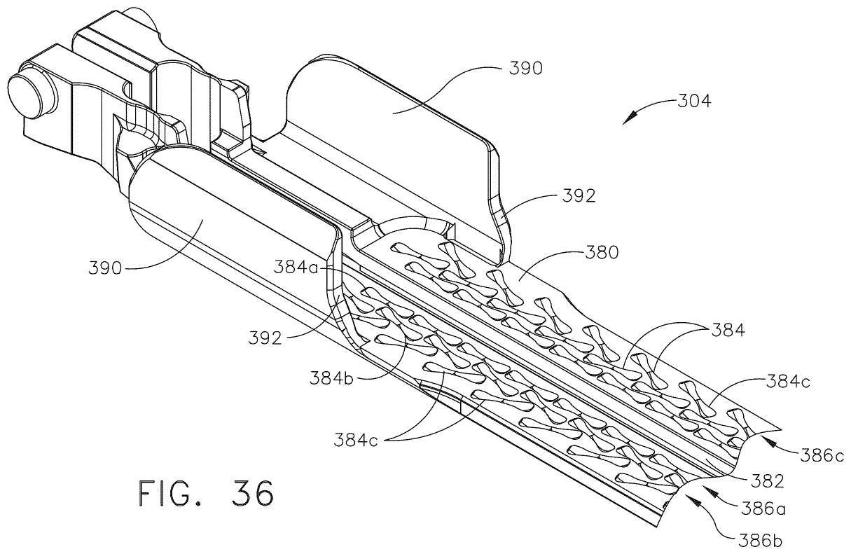

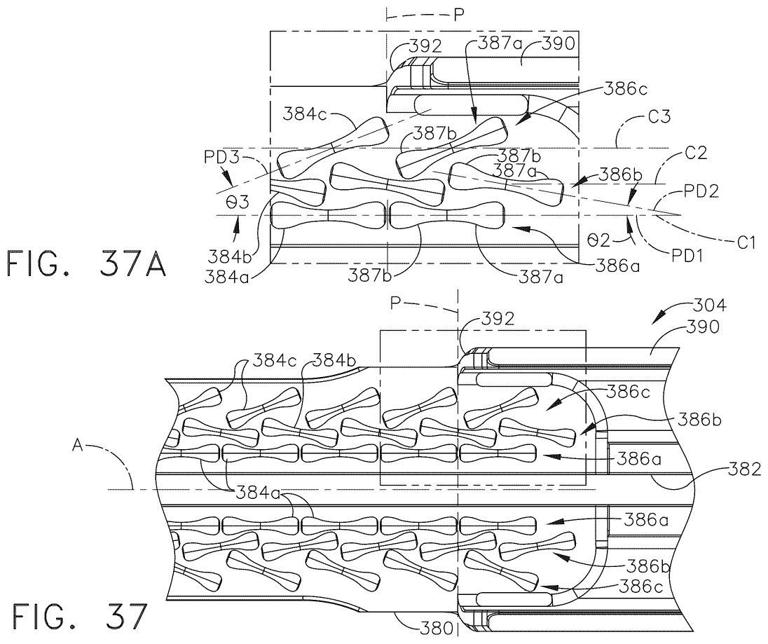

Referring primarily to FIGS. 36, 37 and 37A, an anvil 304 is depicted. The anvil 304 is similar in many aspects to the anvil 204 and various differences are further described herein. The anvil 304 can be used in the surgical stapling assembly 100 (FIG. 1) and can deform staples ejected from the fastener cartridge assembly 206 in various instances. The anvil 304 includes an anvil forming surface 380 with a longitudinal slot 382 and forming pockets 384 defined therein. The forming pockets 384 are arranged in multiple rows 386 (i.e. inner forming pockets 384a in an inner row 386a, intermediate forming pockets 384b in an intermediate row 386b, and outer forming pockets 384c in an outer row 386c) on either side of the longitudinal slot 382. The longitudinal slot 382 is adapted to receive an upright portion of a firing member, such as an upper portion and upper pins or flanges on an I-beam firing member, for example. The forming pockets 384 are configured to receive the legs of the fasteners and the guide the fastener legs along a predefined curvature into a formed configuration.

In other instances, the forming surface 380 can include additional rows of forming pockets or fewer rows of forming pockets on one or both sides of the longitudinal slot 382. In certain instances, the forming surface 380 can be symmetrical about a longitudinal axis A and the rows of forming pockets can be symmetrical about the longitudinal axis A. In other instances, the rows of forming pockets can be asymmetrical about the longitudinal axis A.

The inner forming pockets 384a in the anvil 304 are longitudinally-aligned with the longitudinal axis A, i.e. extend along a first proximal-to-distal axis PD1, or pocket centerline, that is parallel, or substantially parallel, to the longitudinal axis A. The intermediate forming pockets 384b and the outer forming pockets 384c are obliquely-oriented relative to the longitudinal axis A. The intermediate forming pockets 384b each define a second proximal-to-distal axis PD2, or pocket centerline, from the proximal end to the distal end thereof, and the second proximal-to-distal axes PD2 are angled away from the longitudinal axis A. The outer forming pockets 384c each define a third proximal-to-distal axis PD3, or pocket centerline, from the proximal end to the distal end thereof, and the PD3 axes are angled toward the longitudinal axis A. The longitudinal alignment of the inner forming pockets 384a and the angled orientations of the intermediate forming pockets 384b and the outer forming pockets 384c allow the intermediate forming pockets 384b to nest closely between the inner forming pockets 384a and the outer forming pockets 384c. The nested arrangement of forming pockets 384 positioned at different angular orientations can provide a compact arrangement of forming pockets and a correspondingly tight staple line, while still allowing sufficient stretch to accommodate movement of the stapled tissue. Various exemplary dimensions are further described herein, which enable the array of drivers, staples, forming-pockets, and so on to fit within the small form factor of a fastener cartridge assembly for minimally-invasive surgeries such as minimally-invasive thoracic surgery, for example.

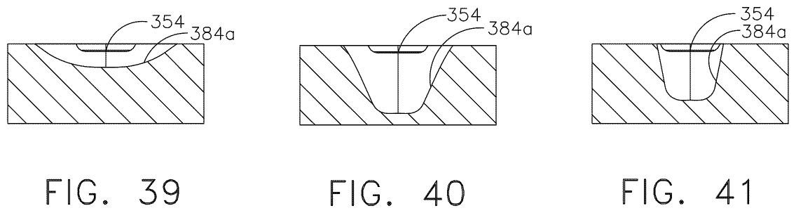

The second proximal-to-distal axes PD2 are oriented at a second pocket angle θ2 relative to the longitudinal axis A, and the third proximal-to-distal axes PD3 are oriented at a third pocket angle θ3 relative to the longitudinal axis A. The third pocket angle θ3 is greater than the second pocket angle θ2. For example, the second pocket angle θ2 can be between 8 degrees and 12 degrees (e.g. approximately 10 degrees), and the third pocket angle θ3 can be between 18 degrees and 25 degrees (e.g. approximately 21.5 degrees). In various instances, the angle of the proximal-to-distal axes PD1, PD2, PD3 of the forming pockets 384 can match or be equal to the angle of the proximal-to-distal axes PD1, PD2, PD3 of the staple cavities 214 (and staples 220 therein). Both the second and third pocket angles θ2, θ3 are greater than the angle at which the first forming pockets 384a are oriented relative to the longitudinal axis. For example, where the first forming pockets 384a are parallel to the longitudinal axis A, the angle of the first forming pockets 384a relative to the longitudinal axis A can be zero. In various instances, the angular orientation of proximal-to-distal axes defined between proximal and distal end 350, 352 (FIG. 39) of the forming pockets 384 can define an increasing gradient from the longitudinal centerline outbound toward the lateral sides of the anvil 204.

A central axis defines each row 386 of forming pockets 384. The central axes extend through a pocket center 354 of each forming pocket 384 in the respective row. More specifically, an inner central axis Cl extends through the pocket centers 354 of each inner forming pocket 384a. The inner central axis C1 is collinear with the first proximal-to-distal axes PD1. An intermediate central axis C2 extends through the pocket centers 354 of each intermediate forming pocket 384b. The intermediate central axis C2 is parallel to the longitudinal axis A and transects the second proximal-to-distal axes PD2 at the angle θ2. An outer central axis C3 extends through the pocket centers 354 of each outer forming pocket 384c. The outer central axis C3 is parallel to the longitudinal axis A and transects the third proximal-to-distal axes PD3 at the angle θ3.

Referring primarily to FIG. 37A, each forming pocket 384 includes a pair of cups (e.g. a proximal cup 387a and a distal cup 387b) configured to receive the staple legs. The inner forming pockets 384a, the intermediate forming pockets 384b, and the outer forming pockets 384c define the same geometry, but are oriented at different angles row-to-row. Consequently, the proximal cups 387a define the same geometry (but different orientations row-to-row) and the distal cup 387b define the same geometry (but different orientations row-to-row). Moreover, each proximal cup 387a is a mirror image reflection of the distal cup 387b across a plane bisecting the forming pocket 384 at the pocket center 354. The geometry of an inner forming pocket 384a is further described herein, but the reader will appreciate the following description of inner forming pocket 384a applies to intermediate forming pockets 384b and 384c, as well. In other aspects of the present disclosure, at least one row of forming pockets can define a different geometry, such as the forming pockets 284b and 284c (FIGS. 26-28A) configured to form non-planar staples, for example.

The proximal cup 387a and the distal cup 387b of each forming pocket 384 are aligned with the proximal-to-distal axis, or pocket axis, of that forming pocket 384. Moreover, the proximal cup 387a and the distal cup 387b of each forming pocket 384 are connected at a centrally-located neck which defines the pocket center 354. The proximal-to-distal axes PD1, PD2, PD3 extend through the pocket center 354 of each forming pocket 384 and bisect each cup 387a, 387b of the forming pocket 384.

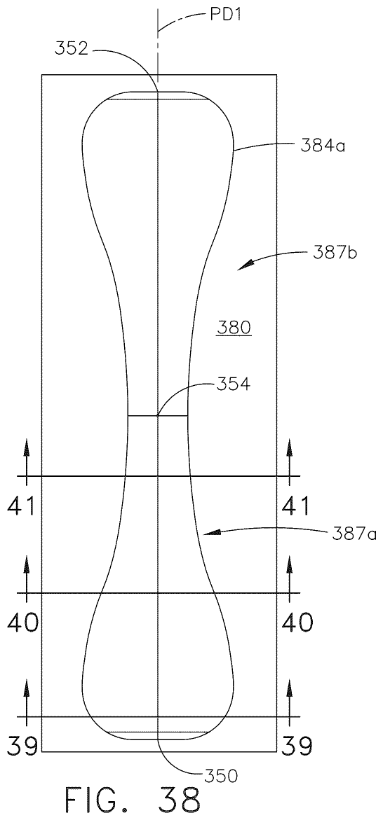

Referring primarily to FIGS. 38-41, the inner forming pocket 384a includes the proximal cup 387a, which is configured to receive a proximal staple leg, and the distal cup 387b, which is configured to receive a distal staple leg of the same staple. The staple legs can be directed along the entry ramp of each inner cup 387a, 387b, which converge toward the pocket center 354, and along the first proximal-to-distal axis PD1 thereof. The inner forming pocket 384a is structured to bend the staple legs at the apex of each proximal and distal cup 387a, 387b, and an exit ramp of each proximal and distal cup 387a, 387b is structured to further deform the staple legs back toward the fastener cartridge assembly 206 (FIG. 1) and along the first proximal-to-distal axis PD1 thereof. The inner forming pockets 384a are structured to deform the staples into a planar, or substantially planar, configuration, in which the staple legs and base are aligned with a plane through the first proximal-to-distal axis PD1 thereof.

The proximal cup and distal cup 387a, 387b define a teardrop shape extending away from the pocket center 354. More specifically, the boundary of the forming pocket 384a defines a contoured boundary line where the proximal cups 387a is widest near the proximal end 350 and the distal cup 387b is widest near the distal end 352. The cups 387a, 387b taper inward toward the neck (i.e., the pocket center 354) at which the cups 387a, 387b define their narrowest width. Referring now to the cross-sectional views in FIGS. 39-41, the proximal cup 387a is shallowest near the proximal end 350 and the distal cup 387b is shallowest near the distal end 352. From the shallow basin geometry shown in FIG. 39, the proximal and distal cups 387a, 387b become deeper and the walls transition to a straighter, less contoured shape toward the pocket center 354 as shown in FIG. 40. The walls of the inner forming pocket 384a at the intermediate location shown in FIG. 40 define a modified V-shape channel with a flat bottom. Further toward the pocket center 354, referring now to FIG. 41, the walls of the inner forming pocket 384a transition to a more upright orientation as the cups 387a, 387b continue to narrow at the neck connecting the proximal and distal cups 387a, 387b.

In various aspects of the present disclosure, the foregoing description of the inner forming pocket 384a can also apply to the inner forming pocket 284a (FIGS. 26-28A), for example. Notably, both forming pockets 284a and 384a include connected cups that are symmetrical about the proximal-to-distal axis and a transverse axis that bisects the proximal-to-distal axis at the pocket center.

Referring again to FIG. 37A, in various instances, the distance between the central inner axis C1 and the central intermediate axis C2 can be less than the distance between the central intermediate axis C2 and the central outer axis C3. For example, the distance between the central inner axis C1 and the central intermediate axis C2 can be less than the width of the forming pockets. Additionally or alternatively, the distance between the central intermediate axis C2 and the central outer axis C3 can be greater than the width of the forming pockets 384.

In various aspects of the present disclosure, the portion of the forming surface 380 filled with forming pockets 384 is maximized within the constraints imposed by how close the forming pockets can be made during a manufacturing process. For example, as further described herein with respect to anvil 304, 35% to 55% of the total tissue-contacting surface of the forming surface 380 can be within the boundaries of a forming pocket.

Referring again to FIGS. 37 and 38, the anvil 304 further comprises tissue stops 390 projecting away from the forming surface 380. Each tissue stop 390 can include an upright distal-facing edge 392 defining a tissue stop plane P, as further described herein. Referring primarily to FIGS. 37 and 37A, one whole inner forming pocket 384a, one whole intermediate forming pocket 384b, and one whole outer forming pocket 384c are positioned proximal to the tissue stop plane P. Moreover, in such instances, at least one longitudinally-oriented staple pocket 384a and two obliquely-oriented staple pockets 384b, 384c are positioned proximal to the tissue stop plane P. In various aspects of the present disclosure, at least one longitudinally-aligned staple 220 (formed by a first forming pocket 384a) and at least one obliquely-oriented staple 220 (formed by a second forming pocket 384b or a third forming pocket 384c) in a staple line can be proximal to the tissue stop plane P.

In various aspects of the present disclosure, a fastener cartridge assembly can utilize different arrangements of staple cavities, drivers, and staples. Moreover, different arrangements of staple cavities, drivers, and staples can necessitate a different arrangement of forming pockets in the anvil opposing the fastener cartridge assembly. Alternative embodiments are further described herein. The reader will appreciate that modifications to the fastener cartridge assembly can necessitate corresponding changes to the anvil.

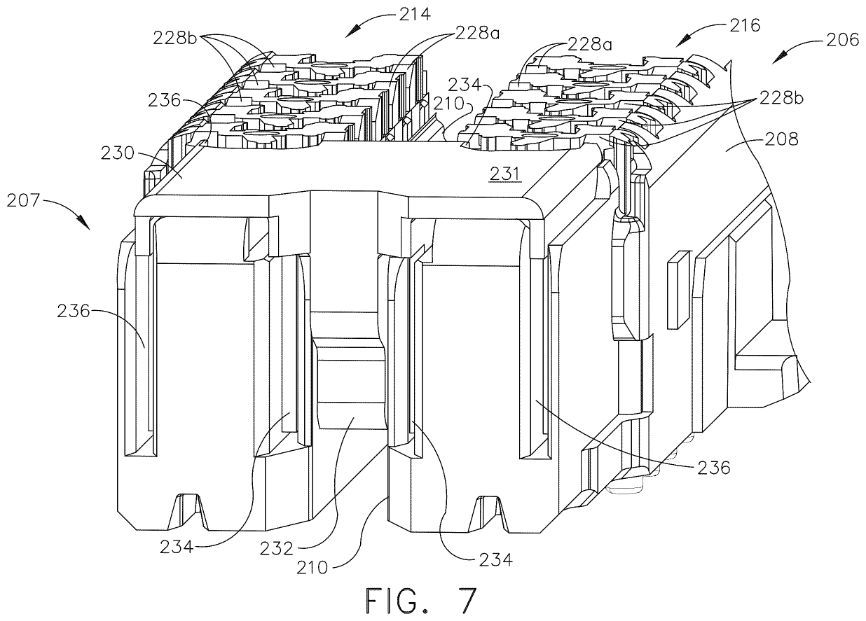

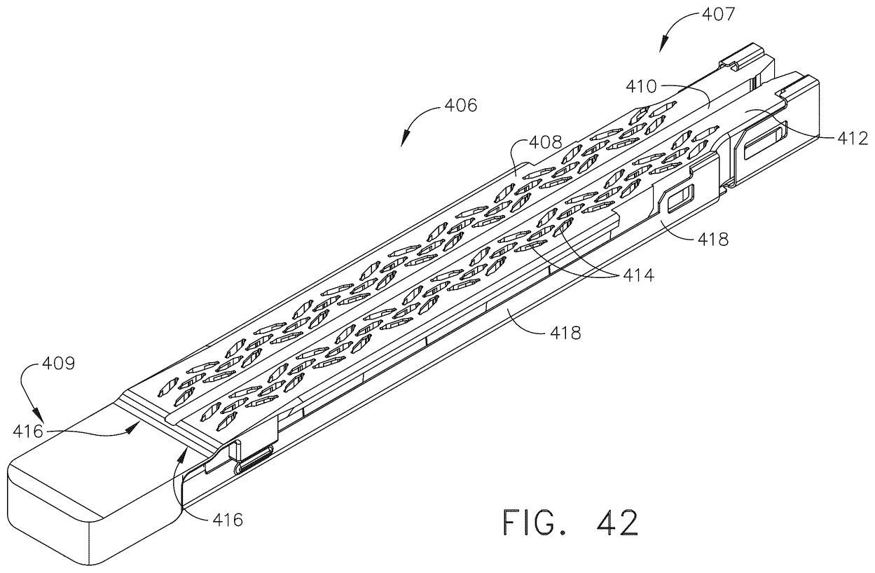

Referring primarily to FIGS. 42-44, a fastener cartridge assembly 406 is shown. The fastener cartridge assembly 406 in similar in many aspects to the fastener cartridge assembly 206 (FIGS. 1-12) and is adapted for use with a surgical stapling assembly (e.g. surgical stapling assembly 100 in FIG. 1) and to receive a translating firing member therethrough to transect tissue and sequentially fire fasteners along both sides of the cutline. The fastener cartridge assembly 406 includes a cartridge body 408 having a longitudinal knife slot 410 defined at least partially through the cartridge body 408 from a proximal end 407 of the cartridge body 408 toward a distal end 409 of the cartridge body 408. The longitudinal knife slot 410 is configured to receive a portion of the firing member therethrough. An upright portion of the firing member can include a cutting edge, for example.

The cartridge body 408 further includes a tissue-supporting deck 412. Cavities 414 are defined into the tissue-supporting deck 412 forming openings therein. The cavities 414 are configured to receive fasteners and guide the fasteners toward and into tissue during a firing motion. The cavities 414 are arranged in a plurality of rows 416.

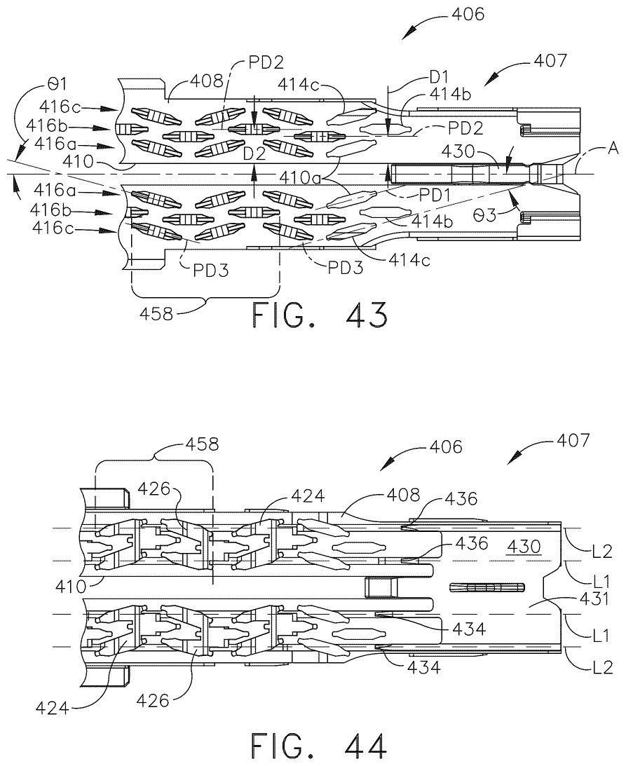

Referring primarily to FIG. 43, the cartridge body 408 includes three rows 416 on both sides of the longitudinal knife slot 410. The rows include an inner row 416a comprised of inner cavities 414a, an intermediate row 416b comprised of intermediate cavities 414b, and an outer row 416c comprised of outer cavities 414c.

In other instances, the cartridge body 408 can include additional rows of cavities or fewer rows of cavities on one or both sides of the longitudinal knife slot 410. The rows 416 of cavities 414 in cartridge body 408 are the same on either side of the longitudinal knife slot 410. In other instances, the fastener cartridge assembly 406 can be symmetrical about a longitudinal axis A and the rows of cavities can be symmetrical about the longitudinal axis A.

The intermediate cavities 414b in the cartridge body 408 are longitudinally-oriented relative to the longitudinal axis A. In other words, second proximal-to-distal axes PD2, which extend along the centerline of the intermediate cavities 414b, are oriented parallel, or substantially parallel, to the longitudinal axis A. The second proximal-to-distal axes PD2 of longitudinally adjacent intermediate cavities 414b, however, are not collinear. Rather, the intermediate cavities 414b are staggered laterally to accommodate the inner cavities 414a and the outer cavities 414c adjacent thereto. More specifically, a first intermediate cavity 414b is spaced a first distance D1 (FIG. 43) from the longitudinal knife slot 410 and an adjacent second intermediate cavity 414b is space a second distance D2 from the longitudinal knife slot 410, where the first distance D1 is different than the second distance D2.

The inner cavities 414a and the outer cavities 414c are obliquely-oriented relative to the longitudinal axis A (and relative to the second proximal-to-distal axes PD2). In other words, the first proximal-to-distal axes PD1, which extend along the centerline of the inner cavities 414a, are oriented at an oblique angle θ1 relative to the longitudinal axis A. Further, the third proximal-to-distal axes PD3, which extend along the centerline of the outer cavities 414c, are oriented at an oblique angle θ3 relative to the longitudinal axis A (and relative to the second proximal-to-distal axes PD2). The inner cavities 414a and the outer cavities 414c are oriented at alternating positive and negative angles relative to the longitudinal axis A. For example, along the inner row 416a, the inner cavities 414a alternate between being oriented at the oblique angle θ1 toward the longitudinal axis A and being oriented at the oblique angle θ1 away from the longitudinal axis A on both sides of the cartridge body 408. Similarly, along the outer row 416c, the outer cavities 414c alternate between being oriented at the oblique angle θ3 toward the longitudinal axis A and being oriented at the oblique angle θ3 away from the longitudinal axis A on both sides of the cartridge body 408. Moreover, aligned inner and outer cavities 414a, 414c on the same side of the longitudinal knife slot 410 (and supported by the same driver 426 or 428, further described herein) are oriented parallel to each other. In other words, the first and third proximal-to-distal axes PD1 and PD3 of inner and outer cavities 414a, 414c, respectively, are parallel to each other but not to second proximal-to-distal axes PD2.

The oblique angles θ1 and θ3 can be between 5 degrees and 25 degrees in various aspects of the present disclosure. In certain aspects, the oblique angles θ1 and θ3 can be the same, and can be around 10 degrees, for example, or around 20 degrees, for example. In the depicted embodiment, the oblique angles θ1 and θ3 are 15 degrees. Alternative angular orientations are also contemplated.

The arrangement of six cavities—two inner cavities 414a, two intermediate cavities 414b, and two outer cavities 414c—forms a longitudinally-repeating pattern 458 along the length (or a portion of the length) of the cartridge body 408. The longitudinally-repeating pattern 458 includes three staples 220 fired by a first driver 424 (FIG. 44) and three staples 220 fired by a second driver 426 (FIG. 44). Each pattern 458 forms a wave, and the sequence of waves undulates laterally along the deck 412 and correspond to a wave-like pattern of formed staples in a staple line.

In various aspects of the present disclosure, the pattern of waves is selected to provide a compact arrangement of staple cavities 414 and corresponding staples 420 to improve homeostasis. Various exemplary dimensions (e.g. angular orientations) are further described herein, which enable the array of drivers, staples, forming-pockets, and so on to fit within the small form factor of a fastener cartridge assembly for minimally-invasive surgeries, for example.

Referring primarily to FIG. 42, the fastener cartridge assembly 406 further comprises a cartridge pan 418 configured to encase a portion of the cartridge body 408. Staples 220 can be removably positioned in the cavities 414 in the cartridge body 408. The staples 220 are movably supported by the first and second drivers 424, 426 in the cartridge body 408; the drivers 424, 426 are triple drivers.

The first drivers 424 have a first geometry corresponding to the outward angle of the inner cavity/inner staple and the outer cavity/outer staple. The second drivers 426 have a second geometry corresponding to the inward angle of the inner cavity/inner staple and the outer cavity/outer staple. First drivers 424 and second drivers 426 alternate along both sides of the cartridge body 408. The cartridge pan 418 is configured to retain the drivers 424, 426 in the cartridge body 408.

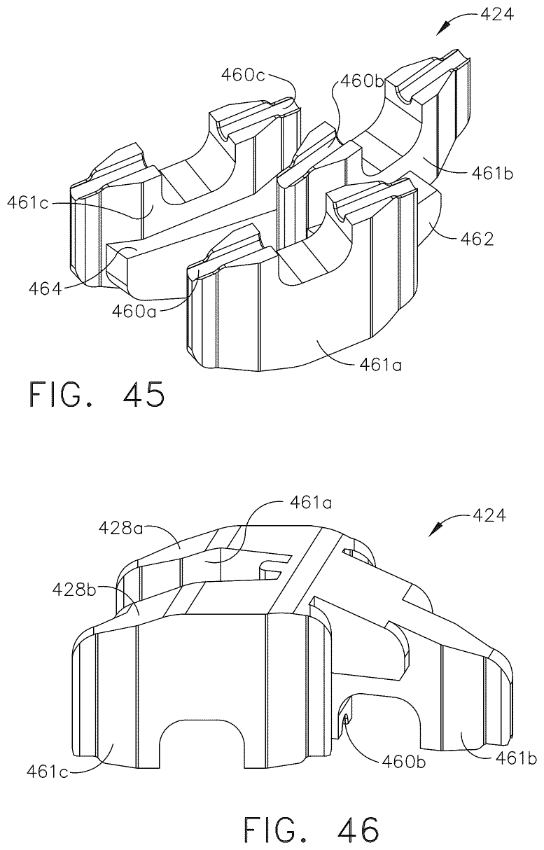

Referring primarily now to FIGS. 45-47, a first driver 424 is shown. The first driver 424 includes three cradles 460a, 460b, and 460c, and each driver 424 is configured to support a staple 220 in the inner row 416a, a staple 220 in the intermediate row 416b, and a staple 220 in the outer row 416c. To support the staples 220 oriented at different angles row-to-row, the triple drivers 424 include columns 461a, 461b, 461c comprising the cradles 460a, 460b, 460c, respectively, oriented at different angles, bridges (or flanges) 462, 464 between the support columns, and ramps (i.e. first ramps 428a and second ramps 428b) (FIG. 46) on the underside thereof. The ramps 428a and 428b are configured to be driven by a sled 430 during the firing stroke. The reader will appreciate that the second drivers 426 are similar in many aspects to the first drivers 424 except for the opposite angular orientation of the inner and outer support columns 461a, 461c (and cradles 460a, 460c thereof) relative to the longitudinal-orientation of the intermediate support column 461b (and cradle 460b thereof).

Referring primarily now to FIG. 44, the fastener cartridge assembly 406 also includes the sled 430 configured to translate distally during a firing motion to engage the drivers 424, 426 and lift the drivers 424, 426 to eject the staples 220 supported thereon from the cartridge body 408. The sled 430 is a four-rail sled having a connecting flange 431, a first pair of rails 434 on a first side of the longitudinal knife slot 410, and a second pair of rails 436 on an second side of the longitudinal knife slot 410. The first pair of rails 434 are longitudinally aligned with each other and longitudinally offset from the second pair of rails 436, which are also longitudinally-aligned with each other.

The underside of the cartridge body 408 is also depicted in FIG. 44, in which the sled 430 is in the proximal, unfired position at the proximal end 407 of the cartridge body 408. The rails 434, 436 are configured to drivingly engage the ramps 428a, 428b on the underside of the drivers 424, 426 (e.g. FIGS. 45-47) to drive the drivers 424, 426 toward the deck 412 (FIG. 42). The rails 434 are aligned with the inner and outer rows 416a, 416c on a first side of the longitudinal knife slot 410 and can pass under the drivers 424, 426 positioned therein, and the rails 436 are aligned with the inner and outer rows 416a, 416c on a second side of the longitudinal knife slot 410 and can pass under the drivers 424, 426 positioned therein.

The columns 461a, 461b, 461c are configured to move through the cavities 414a, 414b, 414c, respectively, during a firing stroke. The sidewalls of the cavities 414 guide the columns 461a, 461b, 461c toward the deck 412 during the firing stroke to eject the staples 220 supported on the cradles 460a, 460b, and 460c. The cradles 460a, 460b, and 460c can be overdriven with respect to the cartridge body 408, as further described herein. Each cradle 460a, 460b, and 460c defines a proximal-to-distal axis PD1, PD2, and PD3, respectively, that corresponds to the proximal-to-distal axis PD1, PD2, PD3 of the staple cavity 414a, 414b, 414c in which it is positioned. More specifically, the inner cradles 460a are oriented along the first proximal-to-distal axes PD1, the intermediate cradles 460b are oriented along the second proximal-to-distal axes PD2, and the outer cradles 460c are oriented along the third proximal-to-distal axes PD3.

The first proximal-to-distal axis PD1 is oriented at a first angle θ1 relative to the longitudinal axis A, the second proximal-to-distal axis PD2 is oriented at a second angle θ2 relative to the longitudinal axis A, and the third proximal-to-distal axis PD3 is oriented at a third angle θ3 relative to the longitudinal axis A. In various instances, the second angle θ2 is zero degrees and the PD1 axis is oriented parallel, or substantially parallel to the longitudinal axis A. Longitudinal alignment of a row of staples can help provide a robust seal along the cutline.

In various instances, the first angle θ1 and the third angle θ3 can be between 5 degrees and 25 degrees. The oblique angles θ1 and θ3 are the same in certain embodiments, and can be around 10 degrees or around 20 degrees. In the depicted embodiment, the first proximal-to-distal axis PD1 and the third proximal-to-distal axis PD3 are parallel, and the first and third angles θ1 and θ3 are 15 degrees corresponding to the angular orientation of the inner and outer staple cavities 414a and 414c.

In other embodiments, the first and third angles θ1 and θ3 can be greater than 15 degrees, and can be between 15 degrees and 25 degrees, for example. In still other embodiments, the first and third angles θ1 and θ3 can be less than 15 degrees, and can be between 5 and 15 degrees, for example. Angling the inner and outer cradles 460a and 460c (and staples thereon) can facilitate tissue flow and create a tighter staple line and improve hemostasis and/or compliance of the staple tissue in certain instances. Moreover, alternating the angular orientation of the inner and outer staples 220 in the staple line can allow further nesting of adjacent first and second drivers 424 and 426 and staples 220 thereon to minimize the spacing between the staples 220. Higher angles can permit further stretching or elongation of the staple line, but can require a wider side-to-side footprint within the compact fastener cartridge assembly may not nest as closely end-to-end. Alternating the angular orientation of the obliquely-oriented staples longitudinally along the staple line can allow a greater proximal-to-distal overlap between staples 220 fired from adjacent first and second drivers 424, 426, for example.

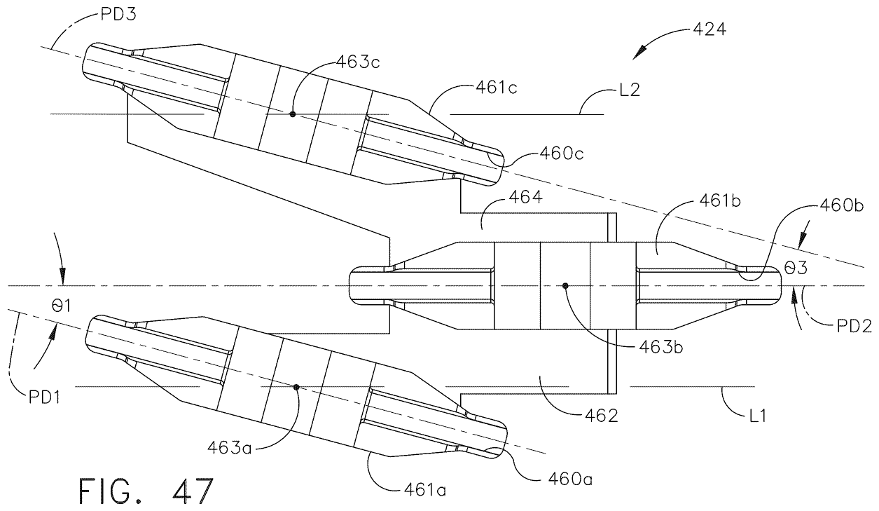

Referring primarily to FIG. 47, the first angle θ1 and the third angle θ3 in the first driver 424 are angled in a first direction, which is toward the longitudinal knife slot 410 when the first drivers 424 are positioned on a first side of the longitudinal knife slot 410 and is away from the longitudinal knife slot 410 when the first drivers 424 are positioned on the opposite side of the longitudinal knife slot 410. Moreover, each cradle 460a, 460b, and 460c includes a geometric center 463a, 463b, and 463c, respectively, through which the proximal-to-distal axes (PD1, PD2, and PD3, respectively) pass through. The first geometric center 463a of the inner cradle 460a is longitudinally-aligned with the third geometric center 463c of the outer cradle 460b; the second geometric center 463b is distal to the first geometric center 463 and the third geometric center 463c.

The first driver 424 includes an inner bridge 462 extending between the inner column 461a and the intermediate column 461b, and also include an outer bridge 464 extending between the intermediate column 461b and the outer column 461c. The bridges 462, 464 connect the columns 461 to form the triple driver spanning three rows of cavities 414 and supporting three staples 220. The rails 434, 436 of the sled 430 are configured to move along the undersides of the driver 424 and ramps 428a, 428b (FIG. 46) thereof during a firing motion.

The first rails 434 are configured to move along a first longitudinal axis L1, which extends through the first geometric centers 463a on one side of the cartridge body 408, and a second longitudinal axis L2, which extends through the third geometric centers 463c on the same side of the cartridge body 408. Similarly, the second rails 436 are configured to move along a first longitudinal axis L1, which extends through the first geometric centers 463a on a second (opposite) side of the cartridge body 408 and a second longitudinal axis L2, which extends through the third geometric centers 463c on the second side of the cartridge body 408. In such instances, the first rails 434 are configured to simultaneously contact and drive the ramps 428a, 428b on the drivers 424, 426 on the first side of the cartridge body 408. Moreover, the second rails 436 are configured to simultaneously contact and drive the ramps 428a, 428b on the drivers 424, 426 on the second side of the cartridge body 408.

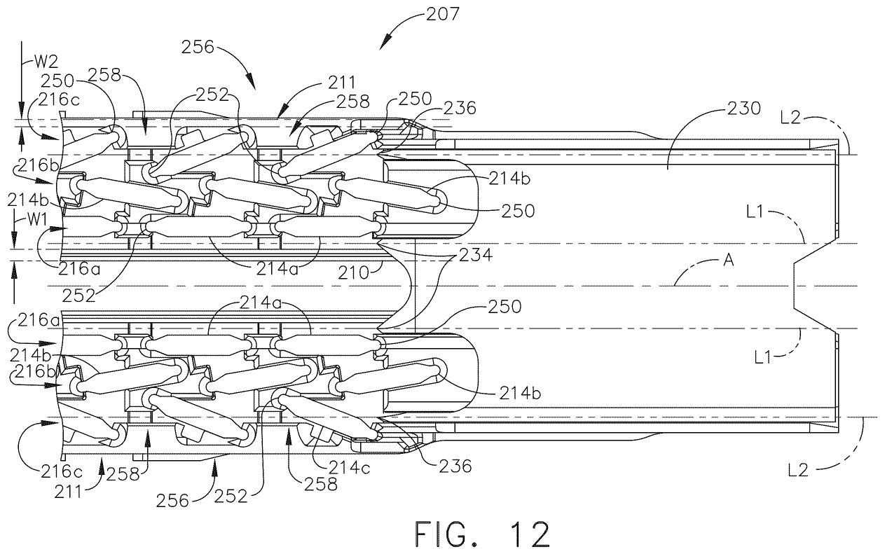

In various aspects of the present disclosure, the drivers 424, 426 can be mass balanced such that the center of mass thereof is equidistantly-spaced from the ramps 428a, 428b, which are driven by the rails 434, 436 during the firing stroke. Additionally or alternatively, the center of mass can be positioned proximal to the leading edge of the ramps 428a, 428b in various aspects of the present disclosure, as further described herein with respect to the drivers 224 and 226 (FIGS. 14 and 15), for example. For example, the length and/or width of the flanges 462, 464 can be adjusted to shift the center of mass to a suitable position.

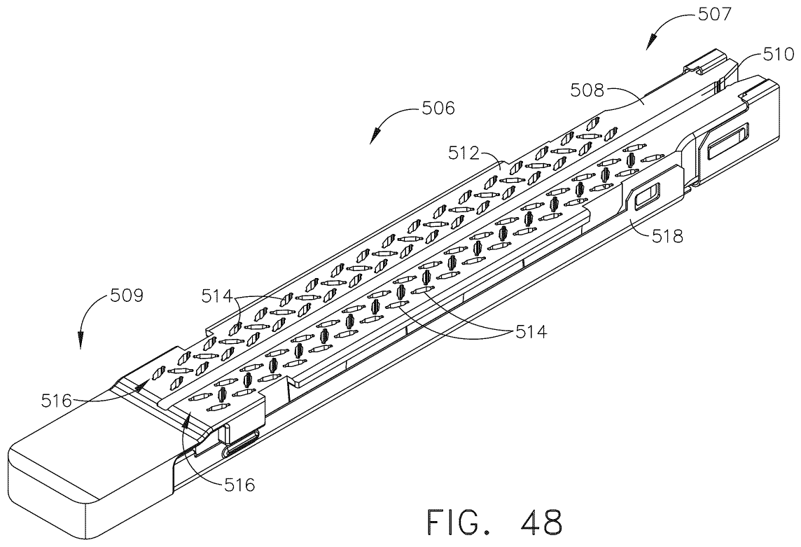

Referring primarily to FIGS. 48-51, a fastener cartridge assembly 506 is shown. The fastener cartridge assembly 506 is similar in many aspects to the fastener cartridge assembly 206 (FIGS. 1-12) and is adapted for use with a surgical stapling assembly (e.g. surgical stapling assembly 100 in FIG. 1) and to receive a translating firing member therethrough to transect tissue and sequentially fire fasteners along both sides of the cutline. The fastener cartridge assembly 506 includes a cartridge body 508 having a longitudinal knife slot 510 defined at least partially through the cartridge body 508 from a proximal end 507 of the cartridge body 508 toward a distal end 509 of the cartridge body 508. The longitudinal knife slot 510 is configured to receive a portion of the firing member therethrough. An upright portion of the firing member can include a cutting edge, for example.

The cartridge body 508 further includes a tissue-supporting deck 512. Cavities 514 are defined into the tissue-supporting deck 512 forming openings therein. The cavities 514 are configured to receive fasteners and guide the fasteners toward and into tissue during a firing motion. The cavities 514 are arranged in a plurality of rows 516.

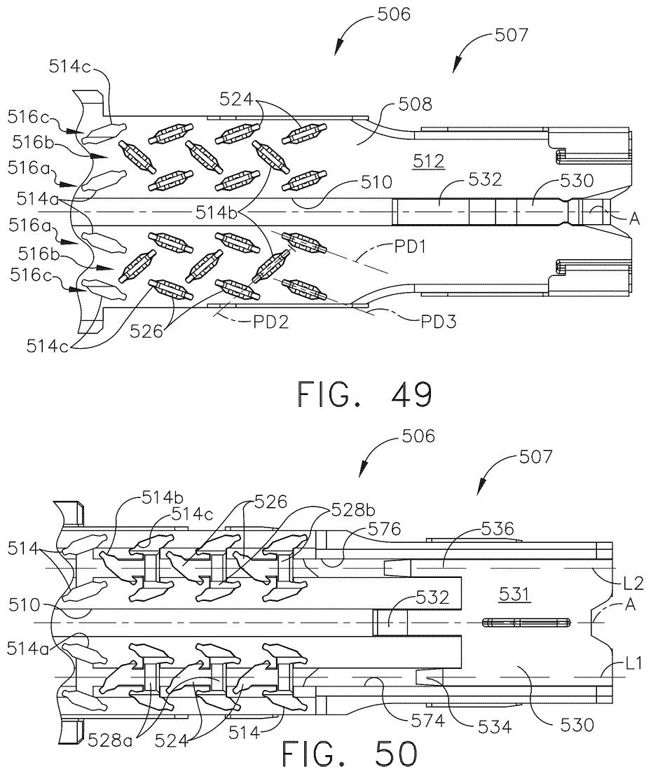

Referring primarily to FIG. 49, the cartridge body 508 includes three rows 516 on both sides of the longitudinal knife slot 510. The rows include an inner row 516a comprised of inner cavities 514a, an intermediate row 516b comprised of intermediate cavities 514b, and an outer row 516c comprised of outer cavities 514c.

In other instances, the cartridge body 508 can include additional rows of cavities or fewer rows of cavities on one or both sides of the longitudinal knife slot 510. The rows 516 of cavities 514 in cartridge body 508 are symmetrical about the longitudinal knife slot 510. In other instances, the fastener cartridge assembly 506 can be asymmetrical about the longitudinal axis A and/or the rows of cavities can be asymmetrical about the longitudinal axis A.

The inner cavities 514a, the intermediate cavities 514b, and the outer cavities 514c are obliquely-oriented relative to the longitudinal axis A. More specifically, the inner cavities 514a define a first proximal-to-distal axis PD1 obliquely-oriented relative to the longitudinal axis A, the intermediate cavities 514b define a second proximal-to-distal axis PD2 obliquely-oriented relative to the longitudinal axis A, and the outer cavities 514c define a third proximal-to-distal axis PD3 obliquely-oriented relative to the longitudinal axis A. The first and third proximal-to-distal axes PD1, PD3 are parallel and the second proximal-to-distal axes PD2 transect the first and third proximal-to-distal axes PD1, PD3. The angular orientations of the cavities 514a, 514b, and 514c correspond to the angular orientations of the support columns 561a, 561b, and 561c (and cradles 560a, 560b, and 560c thereof and staples supported thereon), respectively, which are further described herein.

Referring primarily to FIG. 48, the fastener cartridge assembly 506 further comprises a cartridge pan 518 configured to encase a portion of the cartridge body 508. Staples 220, such as those illustrated in FIG. 3, can be removably positioned in the cavities 514 in the cartridge body 508. The staples 220 are movably supported by the first and second drivers 524, 526 in the cartridge body 508; the first and second drivers 524, 526 are triple drivers.

The first drivers 524 have a first geometry and the second drivers 526 have a second geometry, which is a mirror images of the first geometry owing to the symmetrical arrangement of first drivers 524 on the first side of the cartridge body 508 and the second drivers 526 on the second side of the cartridge body 508. The cartridge pan 518 is configured to retain the first and second drivers 524, 526 in the cartridge body 508.

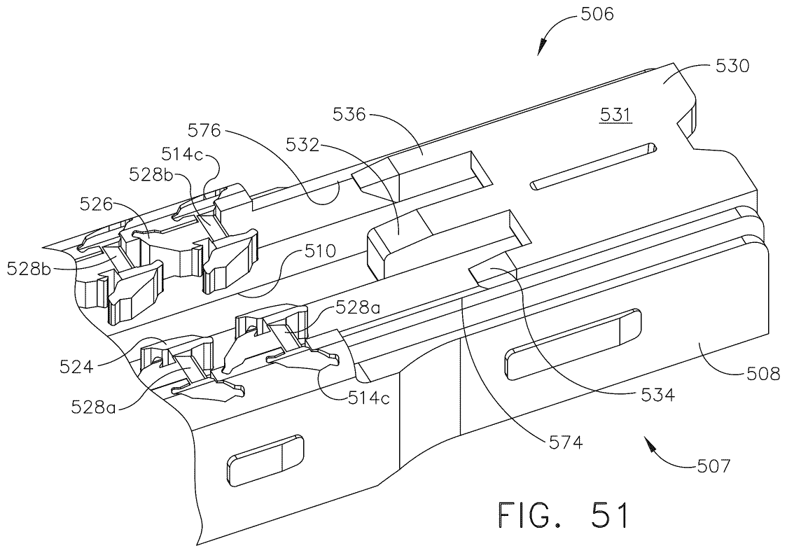

Referring primarily now to FIGS. 50 and 51, the fastener cartridge assembly 506 also includes the sled 530 configured to translate distally during a firing motion to engage the drivers 524, 526 and lift the drivers 524, 526 to eject the staples 220 supported thereon from the cartridge body 508. The sled 530 is a two-rail sled having a connecting flange 531, a first rail 534 on a first side of the longitudinal knife slot 510, and a second rail 536 on an second side of the longitudinal knife slot 510. The first rail 534 is longitudinally offset from the second rail 536. The sled 530 further comprises a central upright 532 positioned intermediate the first rail 534 and the second rail 536 and configured to move through the longitudinal knife slot 510 during the firing motion.

The underside of the cartridge body 508 is also depicted in FIGS. 50 and 51, in which the sled 530 is in the proximal, unfired position at the proximal end 507 of the cartridge body 508. The rails 534, 536 are configured to drivingly engage ramps 528a (of the first drivers 524) and ramps 528b (of the second drivers 526) on the underside of the drivers 524, 526 (e.g. FIGS. 43-45) to drive the drivers 524, 526 toward the deck 512 (FIG. 48). The rails 534 are aligned with the intermediate row 516b on a first side of the longitudinal knife slot 510 and can pass under the drivers 524 positioned therein, and the rails 536 are aligned with the intermediate row 516b on a second side of the longitudinal knife slot 510 and can pass under the drivers 526 positioned therein.

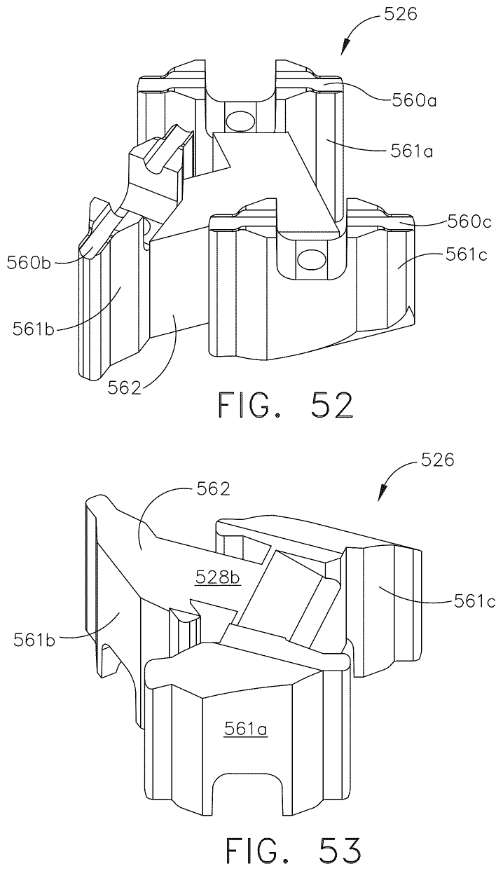

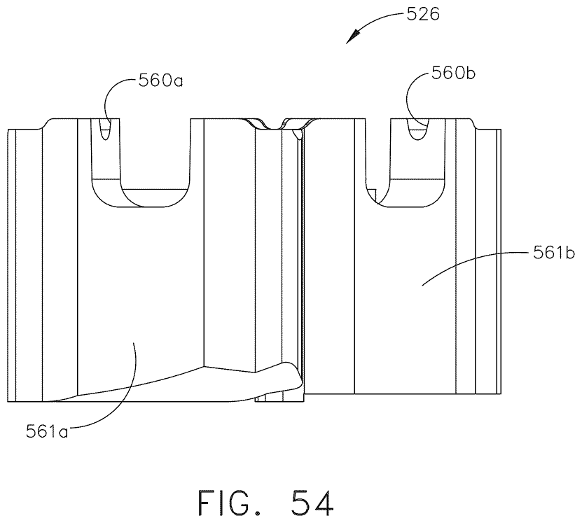

Referring primarily now to FIGS. 52-56, a second driver 526 is shown. The second driver 526 includes three cradles 560a, 560b, and 560c, and each second driver 526 is configured to support a staple 220 in the inner row 516a, a staple 220 in the intermediate row 516b, and a staple 220 in the outer row 516c. To support the staples 220 oriented at different angles row-to-row, the second driver 526 includes columns 561a, 561b, 561c comprising the cradles 560a, 560b, 560c, respectively, oriented at different angles, a bridge (or flange) 562 between the support columns, and a ramp 528b (FIG. 53) on the underside thereof. The ramp 528b is configured to be drivingly engaged and lifted by the sled 530 (FIGS. 50 and 51) during the firing stroke. The reader will appreciate that the first drivers 524 are similar in many aspects to the second drivers 526 except for the mirror image geometry thereof.

The columns 561a, 561b, 561c are configured to move through the cavities 514a, 514b, 514c, respectively, during a firing stroke. The sidewalls of the cavities 514 guide the columns 561a, 561b, 561c toward the deck 512 during the firing stroke to eject the staples 220 supported on the cradles 560a, 560b, and 560c. The cradles 560a, 560b, and 560c can be overdriven with respect to the cartridge body 508, as further described herein. Each cradle 560a, 560b, and 560c defines a proximal-to-distal axis PD1, PD2, and PD3, respectively, that corresponds to the proximal-to-distal axis PD1, PD2, PD3 of the staple cavity 514a, 514b, 514c in which it is positioned. More specifically, the inner cradles 560a are oriented along the first proximal-to-distal axes PD1, the intermediate cradles 560b are oriented along the second proximal-to-distal axes PD2, and the outer cradles 560c are oriented along the third proximal-to-distal axes PD3.

The first proximal-to-distal axis PD1 is oriented at a first angle θ1 relative to the longitudinal axis A, the second proximal-to-distal axis PD2 is oriented at a second angle θ2 relative to the longitudinal axis A, and the third proximal-to-distal axis PD3 is oriented at a third angle θ3 relative to the longitudinal axis A. In the depicted embodiment, the second angle θ2 of the intermediate cradle 560b is greater than the first angle θ1 and the third angle θ3 of the inner cradle 560a and the outer cradle 560c, respectively. For example, the second angle θ2 is double the first and third angles θ1 and θ3.

Where the first and third proximal-to-distal axes PD1 and PD3 are oriented away from the longitudinal axis A, the second proximal-to-distal axis PD2 is oriented toward the longitudinal axis A, and vice versa. The foregoing arrangement along with the varying angular orientations of the cradles 560a, 560b, 560c and staples thereon allows proximal-to-distal nesting of the drivers and overlap of staples 220 in the resultant staple line, which improves sealing and homeostasis in various instances. Moreover, the angled orientation of the staples can provide flexibility and a more compliant staple line in stapled tissue, for example.

In various instances, the first angle θ1 and the third angle θ3 can be between 10 degrees and 30 degrees. The oblique angles θ1 and θ3 are the same in certain embodiments. In the depicted embodiment, the first proximal-to-distal axis PD1 and the third proximal-to-distal axis PD3 are parallel, and the first and third angles θ1 and θ3 are 20 degrees corresponding to the angular orientation of the inner and outer staple cavities 514a and 514c.

In various instances, the second angle θ2 can be between 30 degrees and 50 degrees. In the depicted embodiment, the second angle θ2 is 41 degrees corresponding to the angular orientation of the intermediate staple cavities 514b. In instances in which the second angle θ2 is 41 degrees and the first and third angles θ1, θ3 are 20 degrees, the second angle θ2 is approximately twice the amount of the first and third angles θ1, θ3.

In other aspects of the present disclosure, the first and third angles θ1 and θ3 can be less than 10 degrees or more than 30 degrees. In various aspects, the second angle θ2 can be less than 30 degrees or more than 50 degrees. Moreover, in certain instances, the second angle θ2 can be less than double the first and third angles θ1, θ3, such as 1.5× the first and third angles θ1, θ3, for example, or more than double the first and third angles θ1, θ3, such as 2.5× the first and third angles θ1, θ3, for example.

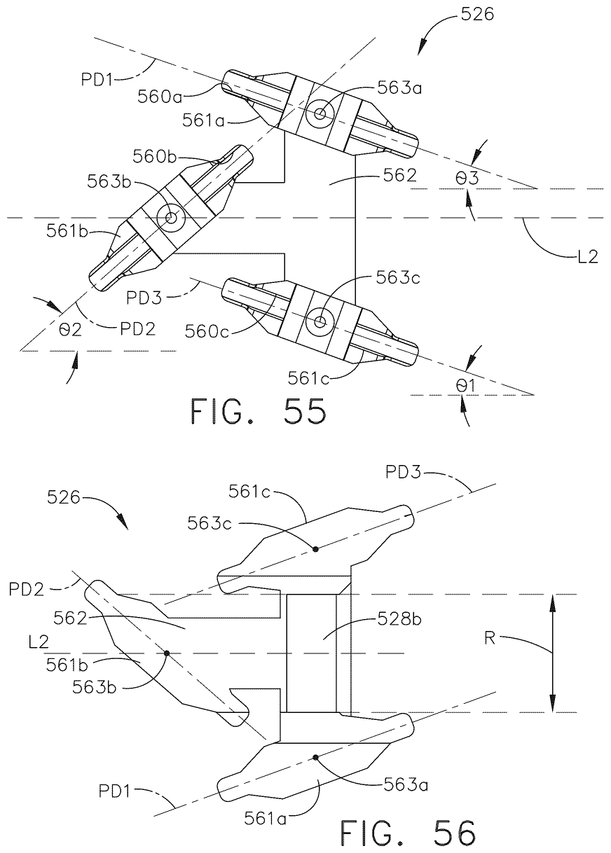

Referring primarily to FIG. 55, the first angle θ1 and the third angle θ3 in the second driver 526 are angled in a first direction, which is toward the longitudinal knife slot 510. Each cradle 560a, 560b, and 560c includes a geometric center 563a, 563b, and 563c, respectively, through which the proximal-to-distal axes (PD1, PD2, and PD3, respectively) pass through. The first geometric center 563a of the inner cradle 560a is longitudinally-aligned with the third geometric center 563c of the outer cradle 560c; the second geometric center 563b is distal to the first geometric center 563a and the third geometric center 563c.

The second driver 526 includes a bridge, or connector, 562 extending between the inner column 561a, the intermediate column 561b, and the outer column 561c. The bridge 562 connects the columns 561 to form the triple driver spanning three rows of cavities 514 and supporting three staples 220. The ramp 528b is positioned on the underside of the bridge 562 and is longitudinally aligned with the intermediate column 561b. Referring primarily to FIG. 56, the ramp 528b defines a ramp width R. The ramp width R can be limited by the space between the ends of the inner and outer columns 461a, 461c though which the sled rail 536 translates during a firing stroke. The ramp width R can be structured to maximize the available space between the inner and outer columns 461a, 461c to provide a greater surface area for driving the second driver 526. The ramp with R can further correspond to the width of channels 574, 576 (FIGS. 50 and 51) along which the rails 534, 536 translate. For example, the channels 574, 576 can be configured to closely receive the rails 534, 536 to guide the rails 534, 536 and prevent binding or jamming during the firing stroke.

In the depicted embodiment, the ramp width R is 0.050 inches. In other instances, the ramp width R can be less than 0.050 inches or more than 0.050 inches. For example, the ramp width R can be between 0.40 inches and 0.60 inches.

Referring again to FIGS. 50 and 51, the rails 534, 536 are configured to move along the undersides of the drivers 524, 526 and ramps 528a, 528b thereof during a firing motion. The first rail 534 is configured to move along a first longitudinal axis L1, which extends through the second geometric centers on one side of the cartridge body 508. Similarly, the second rails 536 are configured to move along a second longitudinal axis L2, which extends through the second geometric centers 563b on a second (opposite) side of the cartridge body 508. Owing to the longitudinal offset between the first rail 534 and the second rail 536 in combination with the longitudinal alignment of the first drivers 524 with corresponding second drivers 526 on the opposite side of the longitudinal knife slot 510, the second drivers 526 are fired before the first drivers 524.

In various aspects of the present disclosure, the drivers 524, 526 can be mass balanced such that the center of mass thereof is equidistantly-spaced from the ramp 528a, 528b thereof which is driven by the rails 534, 536 during the firing stroke. Additionally or alternatively, the center of mass can be positioned proximal to the leading edge of the ramps 528a, 528b in various aspects of the present disclosure, as further described herein with respect to the drivers 224 and 226 (FIGS. 14 and 15), for example. For example, the length and/or width of the bridge 562 can be adjusted to shift the center of mass to a suitable position.

In various aspects of the present disclosure, a fastener cartridge assembly can include obliquely-oriented fasteners that are sequentially-fired from a linear fastener cartridge during a longitudinal firing stroke, as further described herein. In various instances, the obliquely- oriented fasteners can be supported by obliquely-oriented cradles, which are structured and positioned to nest row-to-row and/or proximal-to-distal to consolidate the staple line and provide a tight seal in the stapled tissue. Obliquely-oriented fasteners can improve the stretchability of the stapled tissue, for example, and correspond to a more compliant staple line. In certain instances, obliquely-oriented fasteners can be positioned in an overlapping and crisscrossed arrangement in which one staple base crosses over another staple base. In various instances, overlapping fasteners can provide a tight staple line and prevent air leaks while permitting expansion and contraction of the staple line. Compliant staple lines can be especially important in certain surgical procedures, such as a thoracic transection where the lung repeated inflates and deflates while the tissue heals, for example.

In various instances, a longer staple crown can improve homeostasis. To fit a longer staple crown into the compact footprint of a fastener cartridge assembly while maintaining a high angular orientation relative to the cut line, the staples can be overlaid in the cartridge body 708. For example, as further described herein, overlapping staple crowns, or bases, can be housed in a cartridge body and ejected therefrom by drivers movably supporting the overlapping staple crowns.

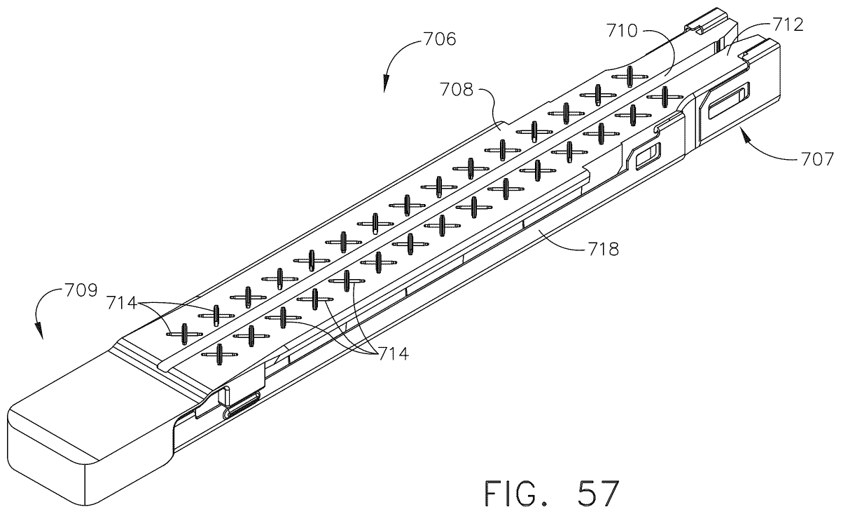

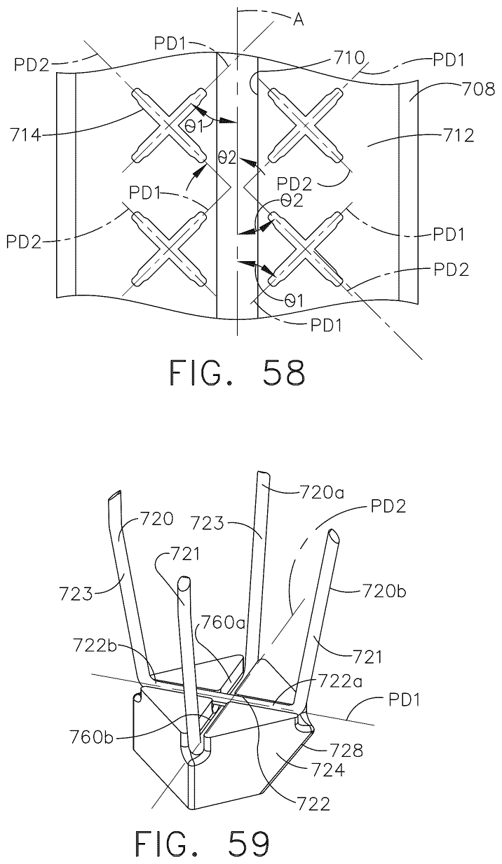

Referring now to FIG. 57, a fastener cartridge assembly 706 is shown. The fastener cartridge assembly 706 in similar in many aspects to the fastener cartridge assembly 206 (FIGS. 1-12) and is adapted for use with a surgical stapling assembly (e.g. surgical stapling assembly 100 in FIG. 1) and is configured to receive a translating firing member therethrough to transect tissue and sequentially fire fasteners along both sides of the cutline. The fastener cartridge assembly 706 includes a cartridge body 708 having a longitudinal knife slot 710 defined at least partially through the cartridge body 708 from a proximal end 707 of the cartridge body 708 toward a distal end 709 of the cartridge body 708. The longitudinal knife slot 710 is configured to receive a portion of the firing member therethrough. An upright portion of the firing member can include a cutting edge, for example.

The cartridge body 708 further includes a tissue-supporting deck 712. Cavities 714 are defined into the tissue-supporting deck 712 forming openings therein. The cavities 714 are X-shaped cavities and configured to receive overlapping staples and to guide the staples toward and into tissue during a firing motion. For example, staples 720 can be positioned in the cavities 714. The staples 720 can be similar in many aspects to the staples 220 (FIG. 3). For example, the staples 720 can be V-shaped staples having a linear base 722, a first leg 721 extending from a first end of the linear base 722, and a second leg 723 extending from the second end of the linear base 722. The first leg 721 and the second leg 723 can splay laterally outward. In various aspects, the staples 720 are configured to be deformed into B-form staples. The B-form staples can define a planar formed shape or a non-planar formed configuration, as further described herein. The bases 722 of the staples 720 in each X-shaped cavity 714 are crossed.

The cartridge body 708 includes a single array of X-shaped cavities 714 on both sides of the longitudinal slot 710. In other instances, the cartridge body 708 can include additional rows of cavities or fewer rows of cavities on one or both sides of the longitudinal knife slot 710. The array of cavities 714 in the cartridge body 708 is symmetrical about the longitudinal knife slot 710. In other instances, the fastener cartridge assembly 706 can be asymmetrical about the longitudinal axis A and/or the cavities 714 can be asymmetrical about the longitudinal axis A.

Each cavity 714 includes a first portion extending along a first proximal-to-distal axis PD1 obliquely-oriented away from the longitudinal axis A, and a second portion intersecting the first portion and extending along a second proximal-to-distal axis PD2 obliquely oriented toward from the longitudinal axis A. The first portion and the second portion intersect or crisscross. The angular orientations of the first proximal-to-distal axes PD1 and the second proximal-to-distal axes PD2 correspond to the angular orientations of the staple-supporting cradles and staples supported thereon, which are further described herein.

Referring primarily to FIG. 57, the fastener cartridge assembly 706 further comprises a cartridge pan 718 configured to encase a portion of the cartridge body 708. Staples 720 (see FIG. 59) can be removably positioned in the cavities 714 in the cartridge body 708. The staples 720 are movably supported by drivers 724 (FIG. 59) positioned in the cartridge body 708; the drivers 724 are double drivers. The cartridge pan 718 is configured to retain the drivers 724 in the cartridge body 708.

A sled having rails, such as the sled 530 (FIGS. 50 and 51), is configured to move through the cartridge body 708 to engage and lift the drivers 724 toward the deck 712. The rails are configured to drivingly engage a ramp 728 on the underside of the drivers 724. The rails can transect one of the proximal-to-distal axes (e.g. the second proximal-to-distal axis PD2 in FIG. 59) and transect the other proximal to distal axes (e.g. the first proximal-to-distal axes PD1 in FIG. 59).

Referring primarily now to FIG. 59, a driver 724 is shown. The driver 724 includes two intersecting cradles 760a and 760b. The cradles 760a and 760b can be overdriven with respect to the cartridge body 708, as further described herein. Each cradle 760a and 760b defines a proximal-to-distal axis PD1 and PD2, respectively, that corresponds to the proximal-to-distal axes PD1 and PD2 of the staple cavity 714 in which it is positioned. More specifically, the cradles 760a are oriented along the first proximal-to-distal axes PD1 away from the longitudinal knife slot 710, and the cradles 760b are oriented along the second proximal-to-distal axes PD2 toward the longitudinal knife slot 710. The cradle 760a, which supports a base 722a of a first staple 720a, is deeper than the cradle 760b, which supports the base 722b of a second staple 720b that crosses over the first staple 720a. The first proximal-to-distal axis PD1 is perpendicular to the second proximal-to-distal axis PD2 in each cavity 714, in various aspects of the present disclosure.

The first proximal-to-distal axis PD1 is oriented at a first angle θ1 relative to the longitudinal axis A, and the second proximal-to-distal axis PD2 is oriented at a second angle θ2 relative to the longitudinal axis A. In the depicted embodiment, the second angle θ2 is equal to the first angle θ1. For example, the first and second angles θ1, θ2 can oriented in opposite directions with respect to the longitudinal axis A. In various instances, the angled orientation of the staples can provide flexibility and a more compliant staple line in stapled tissue, for example.

In various instances, the first angle θ1 and the second angle θ2 can be between 15 degrees and 75 degrees. In various aspects of the present disclosure, the first angle θ1 and the second angle θ2 can be different.

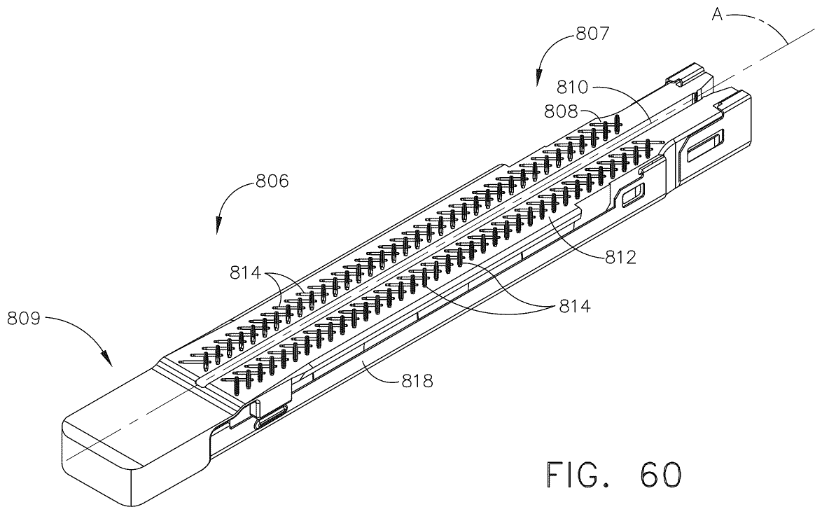

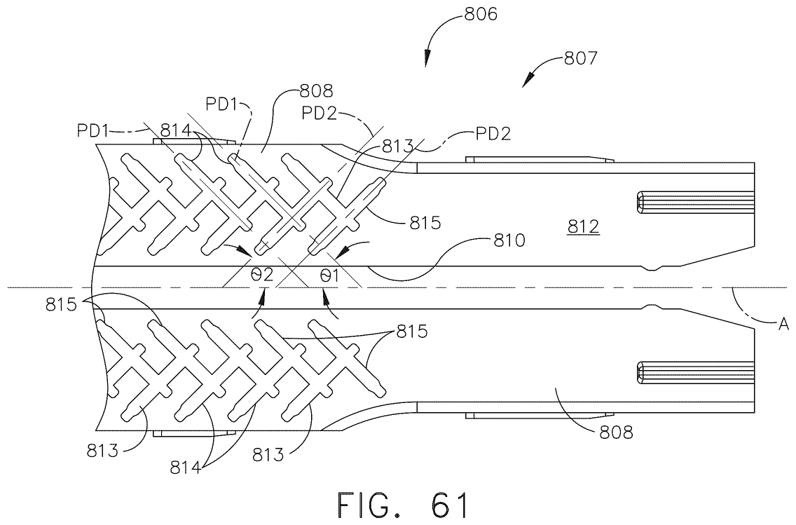

Referring now to FIGS. 60 and 61, a fastener cartridge assembly 806 is shown. The fastener cartridge assembly 806 in similar in many aspects to the fastener cartridge assembly 206 (FIGS. 1-12) and is adapted for use with a surgical stapling assembly (e.g. surgical stapling assembly 100 in FIG. 1) and is configured to receive a translating firing member therethrough to transect tissue and sequentially fire fasteners along both sides of the cutline. The fastener cartridge assembly 806 includes a cartridge body 808 having a longitudinal knife slot 810 defined at least partially through the cartridge body 808 from a proximal end 807 of the cartridge body 808 toward a distal end 809 of the cartridge body 808. The longitudinal knife slot 810 is configured to receive a portion of the firing member therethrough. An upright portion of the firing member can include a cutting edge, for example.

The cartridge body 808 further includes a tissue-supporting deck 812. Intersecting cavities 814 are defined into the tissue-supporting deck 812 forming intersecting openings therein. The intersecting cavities 814 form a crisscross array of intersecting openings laterally and longitudinally along each side of the cartridge body 808. In the depicted arrangement, all of the cavities 814 in the cartridge body 808 are connected by the intersecting, crisscross arrangement thereof.

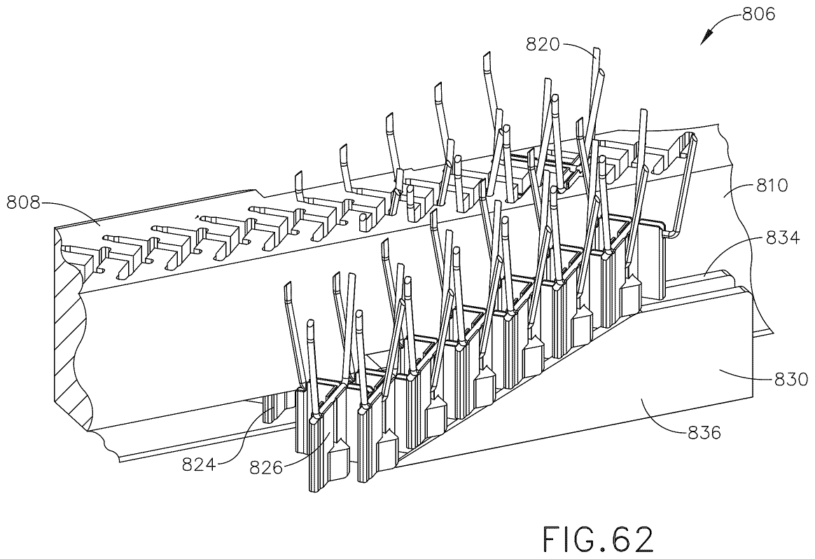

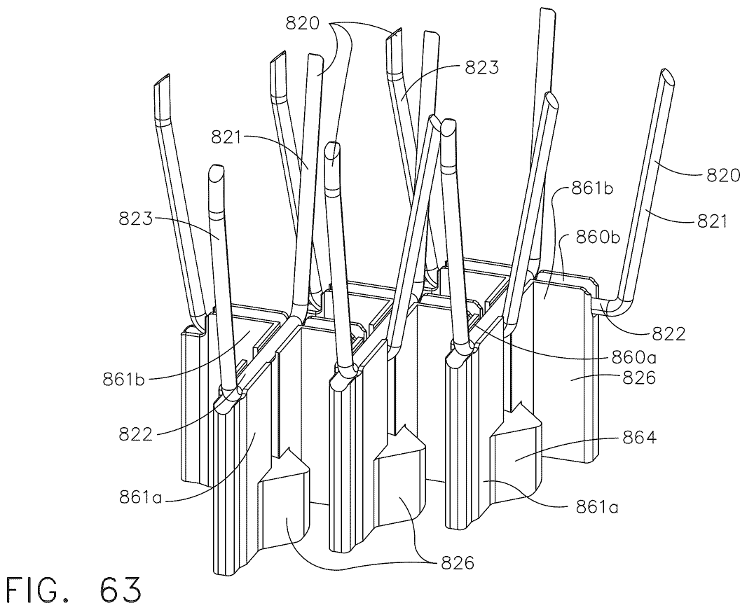

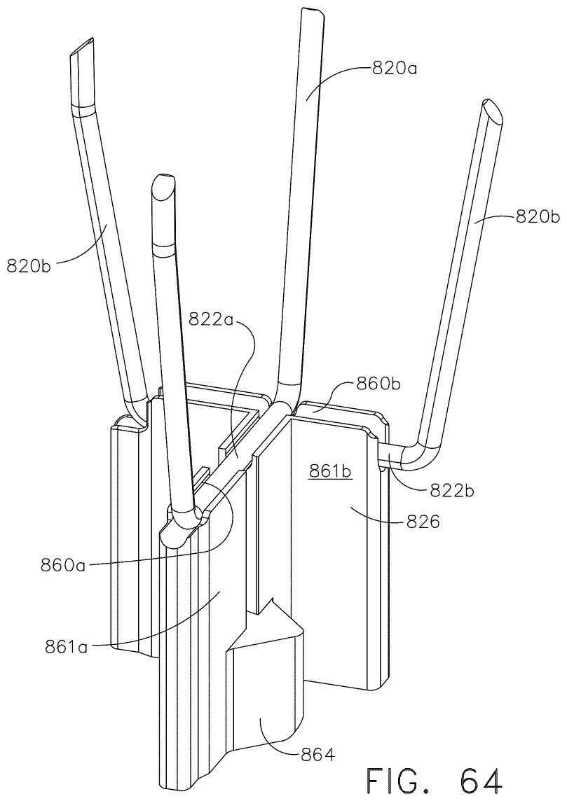

The intersecting cavities 814 are configured to receive overlapping staples and to guide the overlapping staples toward and into tissue during a firing motion. For example, staples 820 shown in FIGS. 62-66 can be positioned in the cavities 814. The staples 820 can be similar in many aspects to the staples 220 (FIG. 3). For example, the staples 820 can be V-shaped staples having a linear base 822, a first leg 821 extending from a first end of the linear base 822, and a second leg 823 extending from the second end of the linear base 822. See FIG. 63. The first leg 821 and the second leg 823 can splay laterally outward. In various aspects, the staples 820 are configured to be deformed into B-form staples. The B-form staples can define a planar formed shape or a non-planar formed configuration, as further described herein. The bases 822 of the staples 820 in the intersecting cavities 814 are crossed. More specifically, the base 822 of each intermediate staple 820 crosses over or under the base 822 of two adjacent staples 820 oriented perpendicular thereto.

The cartridge body 808 includes a connected array of intersecting cavities 814 on both sides of the longitudinal slot 810. In other instances, the cartridge body 808 can include additional rows of cavities on one or both sides of the longitudinal knife slot 810, such as a separate row of cavities holding longitudinally-oriented staples adjacent to the cutline as further described herein, for example. The array of cavities 814 in the cartridge body 808 is symmetrical about the longitudinal knife slot 810. In other instances, the fastener cartridge assembly 806 can be asymmetrical about the longitudinal axis A and/or the cavities 814 can be asymmetrical about the longitudinal axis A.

The intersecting cavities 814 include a first portion 813 extending along a first proximal-to-distal axis PD1 obliquely-oriented away from the longitudinal axis A, and a second portion 815 perpendicular to the first portion 813 and extending along a second proximal-to-distal axis PD2 obliquely-oriented toward the longitudinal axis A. The first portion 813 and the second portion 815 intersect or crisscross along the length of the cartridge body 808 to connect the intersecting cavities 814 along the length (or a portion of the length) thereof. Each first portion 813 intersects a pair of adjacent second portions 815 along an intermediate portion of the cartridge body 808. Similarly, each second portion 815 intersects a pair of adjacent first portions 813 along an intermediate portion of the cartridge body 808. The angular orientations of the first proximal-to-distal axes PD1 and the second proximal-to-distal axes PD2 correspond to the angular orientations of the staple-supporting cradles and staples 820 supported thereon, which are further described herein.

Referring primarily to FIG. 60, the fastener cartridge assembly 806 further comprises a cartridge pan 818 configured to encase a portion of the cartridge body 808. Staples 820 (sec FIGS. 62-66) can be removably positioned in the cavities 814 in the cartridge body 808. The staples 820 are movably supported by drivers 824, 826 (FIG. 62) positioned in the cartridge body 808; the drivers 824, 826 are double drivers. The cartridge pan 818 is configured to retain the drivers 824, 826 in the cartridge body 808.

Referring primarily to FIG. 62, the fastener cartridge assembly 806 also includes a sled 830 configured to move through the cartridge body 808 to engage and lift the drivers 824, 826 toward the deck 812. The sled includes rails 834, 836 that are configured to drivingly engage ramps on the underside of the drivers 824, 826. As further described herein, the rails 834, 836 are configured to move through channel slots in the cartridge body 808 and drivingly engage the drivers during a firing stroke.

Referring primarily now to FIGS. 62-66, an array of second drivers 826 is shown. The reader will appreciate that the first drivers 824 are similar in many aspects to the second drivers 826 except for the mirror image geometry thereof. More specifically, the first drivers 824 have a first geometry and the second drivers 826 have a second geometry, which is a mirror image of the first geometry owing the symmetrical arrangement of first drivers 824 on the first side of the cartridge body 808 and the second drivers 826 on the second side of the cartridge body 808. Each second driver 826 includes two intersecting cradles 860a and 860b. The cradles 860a and 860b can be overdriven with respect to the cartridge body 808, as further described herein. Each cradle 860a and 860b defines a proximal-to-distal axis PD1 and PD2, respectively, that corresponds to the proximal-to-distal axes PD1 and PD2 of the portion 813, 815 of the staple cavity 814 in which it is positioned. More specifically, the cradles 860a are oriented along the first proximal-to-distal axes PD1 away from the longitudinal knife slot 810, and the cradles 860b are oriented along the second proximal-to-distal axes PD2 toward the longitudinal knife slot 810. The cradle 860b, which supports a base 822b of a second staple 820b, is deeper than the cradle 860a, which supports the base 822a of a first staple 820a that crosses over the second staple 820b. The first proximal-to-distal axes PD1 are perpendicular to the second proximal-to-distal axes PD2, in various aspects of the present disclosure.

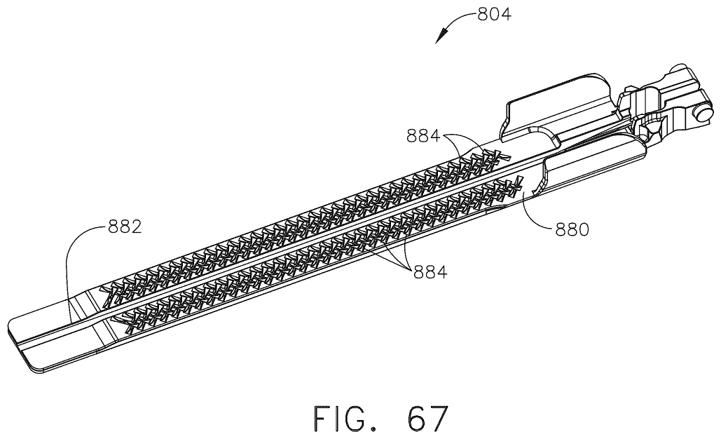

The first proximal-to-distal axis PD1 is oriented at a first angle θ1 relative to the longitudinal axis A (and relative to the longitudinal axis L1 in FIG. 66), and the second proximal-to-distal axis PD2 is oriented at a second angle θ2 relative to the longitudinal axis A (and relative to the longitudinal axis L2 in FIG. 66). In the depicted embodiment, the second angle θ2 is equal to the first angle θ1. For example, the first and second angles θ1, θ2 can be 45 degrees, but oriented in opposite directions with respect to the longitudinal axis A. In various instances, the angled orientation of the staples can provide flexibility and a more compliant staple line in stapled tissue, for example.

In other aspects of the present disclosure, the first angle θ1 and the second angle θ2 can be between 15 degrees and 75 degrees. In various aspects of the present disclosure, the first angle θ1 and the second angle θ2 can be different and may transect at an oblique angle, for example.

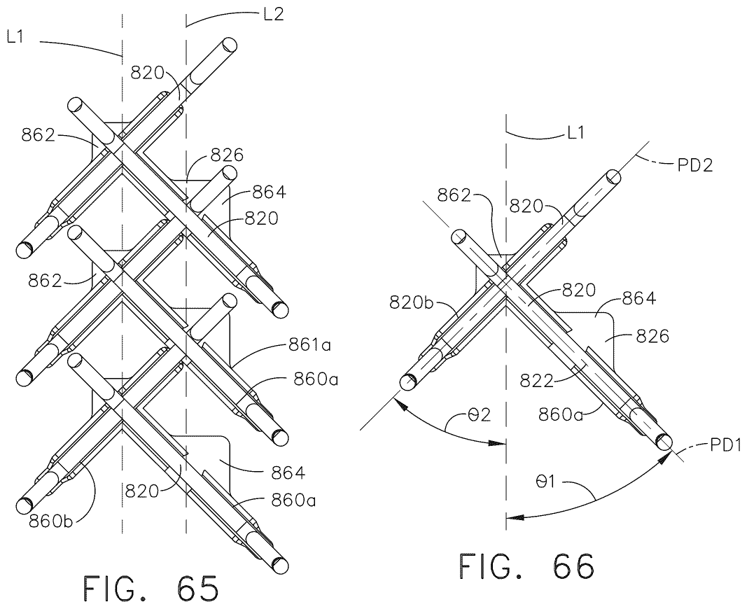

Referring primarily to FIG. 63, the cradles 860a and 860b are supported by columns 861a, 861b respectively. The columns 861a, 861b of each driver 826 cross to form an asymmetrical X-shape (see FIG. 66). The drivers 826 further comprise flanges 862 and 864, which extend from the columns 861a, 861b to form an extended support surface for the ramps on the underside of the drivers 826. Referring primarily to FIG. 65, the rails 834, 836 of the sled 830 are configured to move along longitudinal axes L1, L2, respectively, which extend through the flanges 862, 864, respectively. In various aspects of the present disclosure, the longitudinal axis L1 and L2 along which the sled rails 834 and 836 translates, can pass under the intersection location of the crossed staples 820 supported by the drivers 826.

In instances in which the fastener cartridge assembly includes overlaid staples, the anvil can also be adapted to include overlapping forming pockets that are adapted to receive the staples ejected therefrom.

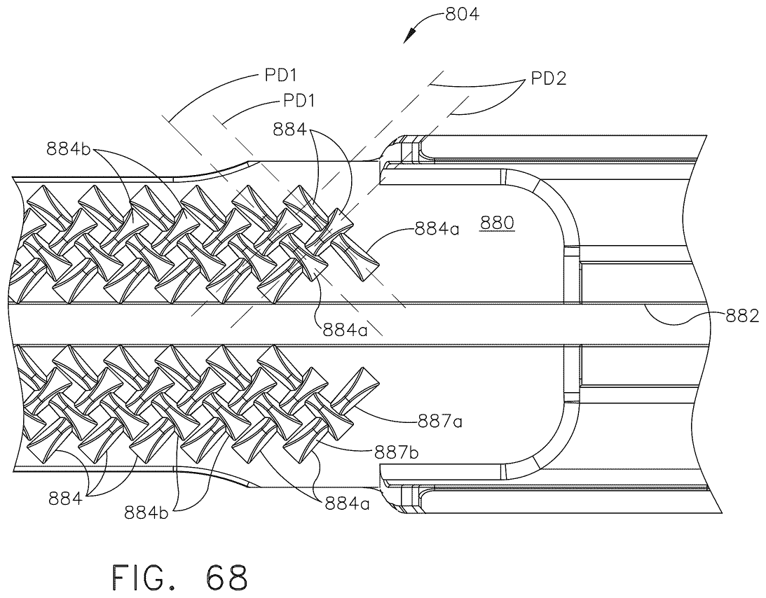

Referring primarily to FIGS. 67 and 68, an anvil 804 is depicted. The anvil 804 is similar in many aspects to the anvil 204 (FIG. 1) and various differences are further described herein. The anvil 804 can be used in with the fastener cartridge assembly 806 (FIG. 60), for example. The anvil 804 includes an anvil forming surface 880 with a longitudinal slot 882 and forming pockets 884 defined therein. The forming pockets 884 form a crisscross array on either side of the longitudinal slot 882. The longitudinal slot 882 is adapted to receive an upright portion of a firing member, such as an upper portion and upper pins or flanges on an I-beam firing member, for example. The forming pockets 884 are configured to receive the legs of the fasteners and guide the fastener legs along a predefined curvature into a formed configuration.

The forming pockets 884 include first forming pockets 884a extending along first proximal-to-distal axes PD1 corresponding to staples 820 in the first portion 813 of the cavities 814 (FIG. 61) and second forming pockets 884b extending along second proximal-to-distal axes PD2 corresponding to staples 820 in the second portion 815 of the cavities 814. The forming pockets 884a, 884b are structured to deform the staples into a planar, or substantially planar, configuration, in which the staple legs and base are aligned with a plane through the proximal-to-distal axis thereof.

Each forming pocket 884 includes a pair of cups (e.g. a proximal cup 887a and a distal cup 887b) configured to receive the staple legs. Each proximal cup 887a is a mirror image reflection of the distal cup 887b across a plane bisecting the forming pocket 884 at the pocket center. The proximal cup 887a and the distal cup 887b of each forming pocket 884 are aligned with the proximal-to-distal axis, or pocket axis, of that forming pocket 884. Moreover, the proximal cup 887a and the distal cup 887b of each forming pocket 884 are connected at a centrally-located neck. The proximal-to-distal axes PD1, PD2 extend through the pocket center of each forming pocket 884 and bisect each cup 887a, 887b of the forming pocket 884. Moreover, the crisscrossing forming pockets 884 transect the crisscrossed forming pocket at the neck thereof.

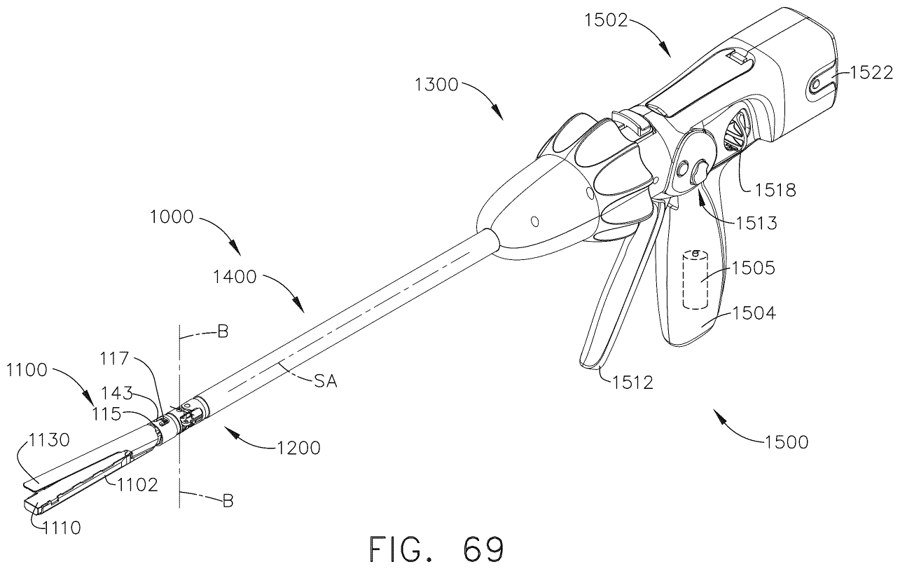

The fastener cartridge assemblies and anvils described herein can be incorporated into a handheld surgical instrument and/or a robotic surgical tool. In various instances, the fastener cartridge assemblies can incorporated into an interchangeable surgical tool assembly 1000 shown in FIG. 69, for example, that is operably coupled to a motor driven handle assembly 1500. The tool assembly 1000 may also be effectively employed with a tool drive assembly of a robotically controlled or automated surgical system. For example, the surgical tool assemblies disclosed herein may be employed with various robotic systems, instruments, components and methods such as, but not limited to, those disclosed in U.S. Pat. No. 9,072,535, entitled SURGICAL STAPLING INSTRUMENTS WITH ROTATABLE STAPLE DEPLOYMENT ARRANGEMENTS, which is hereby incorporated by reference herein in its entirety. The handle assembly 1500, as well as the tool drive assembly of a robotic system may also be referred to herein as “control systems” or “control units”.

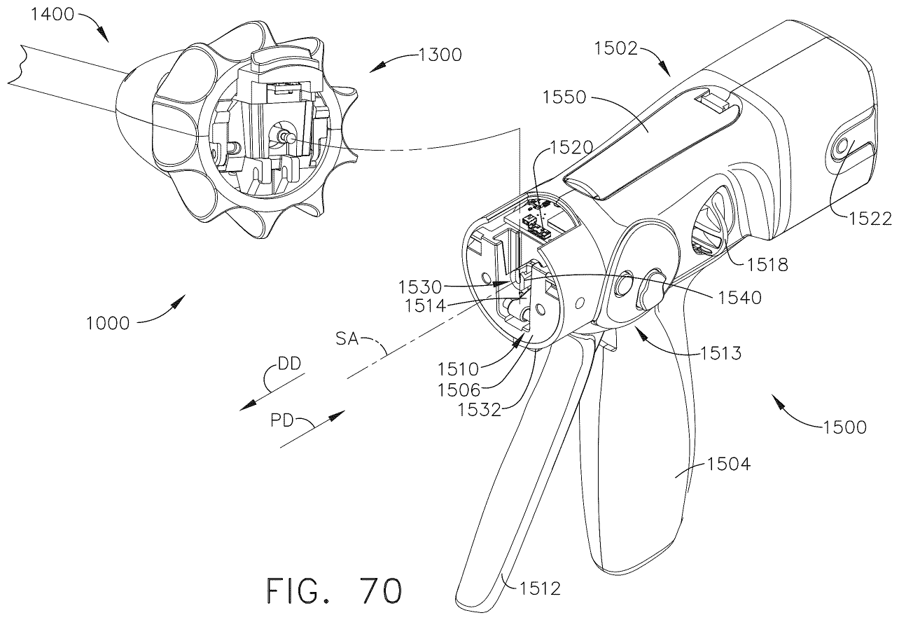

FIGS. 69 and 70 illustrate attachment of the interchangeable surgical tool assembly 1000 to the handle assembly 1500. The handle assembly 1500 may comprise a handle housing 1502 that includes a pistol grip portion 1504 that can be gripped and manipulated by the clinician. The handle assembly 1500 may further include a frame 1506 that operably supports the plurality of drive systems. For example, the frame 1506 can operably support a “first” or closure drive system, generally designated as 1510, which may be employed to apply closing and opening motions to the interchangeable surgical tool assembly 1000 that is operably attached or coupled to the handle assembly 1500. In at least one form, the closure drive system 1510 may include an actuator in the form of a closure trigger 1512 that is pivotally supported by the frame 1506. Such arrangement enables the closure trigger 1512 to be manipulated by a clinician such that when the clinician grips the pistol grip portion 1504 of the handle assembly 1500, the closure trigger 1512 may be easily pivoted from a starting or “unactuated” position to an “actuated” position and more particularly, to a fully compressed or fully actuated position. In various forms, the closure drive system 1510 further includes a closure linkage assembly 1514 that is pivotally coupled to the closure trigger 1512 or otherwise operably interfaces therewith. In use, to actuate the closure drive system 1510, the clinician depresses the closure trigger 1512 toward the pistol grip portion 1504. As described in further detail in U.S. patent application Ser. No. 14/226,142, entitled SURGICAL INSTRUMENT COMPRISING A SENSOR SYSTEM, published as U.S. Patent Application Publication No. 2015/0272575, which is hereby incorporated by reference in its entirety herein, when the clinician fully depresses the closure trigger 1512 to attain a “full” closure stroke, the closure drive system 1510 is configured to lock the closure trigger 1512 into the fully depressed or fully actuated position. When the clinician desires to unlock the closure trigger 1512 to permit it to be biased to the unactuated position, the clinician simply activates a closure release button assembly 1518 which enables the closure trigger 1512 to return to unactuated position. The closure release button assembly 1518 may also be configured to interact with various sensors that communicate with a microcontroller 1520 in the handle assembly 1500 for tracking the position of the closure trigger 1512. Further details concerning the configuration and operation of the closure release button assembly 1518 may be found in U.S. Patent Application Publication No. 2015/0272575.

In at least one form, the handle assembly 1500 and the frame 1506 may operably support another drive system referred to herein as a firing drive system 1530 that is configured to apply firing motions to corresponding portions of the interchangeable surgical tool assembly that is attached thereto. As described in detail in U.S. Patent Application Publication No. 2015/0272575, the firing drive system 1530 may employ an electric motor 1505 (FIG. 69) that is located in the pistol grip portion 1504 of the handle assembly 1500. In various forms, the motor 1505 may be a DC brushed driving motor having a maximum rotation of, approximately, 25,000 RPM, for example. In other arrangements, the motor 1505 may include a brushless motor, a cordless motor, a synchronous motor, a stepper motor, or any other suitable electric motor. The motor 1505 may be powered by a power source 1522 that in one form may comprise a removable power pack. The power pack may support a plurality of Lithium Ion (“LI”) or other suitable batteries therein. A number of batteries, which may be connected in series, may be used as the power source 1522 for the handle assembly 1500. In addition, the power source 1522 may be replaceable and/or rechargeable.

The electric motor 1505 is configured to axially drive a longitudinally movable drive member 1540 in distal and proximal directions depending upon the polarity of the motor. For example, when the motor 1505 is driven in one rotary direction, the longitudinally movable drive member 1540 will be axially driven in the distal direction “DD”. When the motor 1505 is driven in the opposite rotary direction, the longitudinally movable drive member 1540 will be axially driven in a proximal direction “PD”. The handle assembly 1500 can include a switch 1513 which can be configured to reverse the polarity applied to the electric motor 1505 by the power source 1522 or otherwise control the motor 1505. The handle assembly 1500 can also include a sensor or sensors (not shown) that is configured to detect the position of the drive member 1540 and/or the direction in which the drive member 1540 is being moved. Actuation of the motor 1505 can be controlled by a firing trigger 1532 that is pivotally supported on the handle assembly 1500. The firing trigger 1532 may be pivoted between an unactuated position and an actuated position. The firing trigger 1532 may be biased into the unactuated position by a spring (not shown) or other biasing arrangement such that when the clinician releases the firing trigger 1532, it may be pivoted or otherwise returned to the unactuated position by the spring or biasing arrangement. In at least one form, the firing trigger 1532 can be positioned “outboard” of the closure trigger 1512 as was discussed above. As discussed in U.S. Patent Application Publication No. 2015/0272575, the handle assembly 1500 may be equipped with a firing trigger safety button (not shown) to prevent inadvertent actuation of the firing trigger 1532. When the closure trigger 1512 is in the unactuated position, the safety button is contained in the handle assembly 1500 where the clinician cannot readily access it and move it between a safety position preventing actuation of the firing trigger 1532 and a firing position wherein the firing trigger 1532 may be fired. As the clinician depresses the closure trigger 1512, the safety button and the firing trigger 1532 may pivot down wherein they can then be manipulated by the clinician.

In at least one form, the longitudinally movable drive member 1540 may have a rack of teeth (not shown) formed thereon for meshing engagement with a corresponding drive gear arrangement (not shown) that interfaces with the motor 1505. Further details regarding those features may be found in U.S. Patent Application Publication No. 2015/0272575. At least one form also includes a manually-actuatable “bailout” assembly that is configured to enable the clinician to manually retract the longitudinally movable drive member 1540 should the motor 1505 become disabled. The bailout assembly may include a lever or bailout handle assembly that is stored within the handle assembly 1500 under a releasable door 1550. The lever is configured to be manually pivoted into ratcheting engagement with the teeth in the drive member 1540. Thus, the clinician can manually retract the drive member 1540 by using the bailout handle assembly to ratchet the drive member 1540 in the proximal direction “PD”. U.S. patent application Ser. No. 12/249,117, entitled POWERED SURGICAL CUTTING AND STAPLING APPARATUS WITH MANUALLY RETRACTABLE FIRING SYSTEM, published as U.S. Patent Application Publication No. 2010/0089970, the entire disclosure of which is hereby incorporated by reference herein, discloses bailout arrangements and other components, arrangements and systems that may also be employed with the tool assembly 1000.

The interchangeable surgical tool assembly 1000 includes a shaft mounting portion 1300 that is operably attached to an elongate shaft assembly 1400. A surgical end effector 1100 that comprises an elongate channel 1102 that is configured to operably support a staple cartridge 1110 therein is operably attached to the elongate shaft assembly 1400. Alternative staple cartridge assemblies are further described herein. The end effector 1100 may further include an anvil 1130 that is pivotally supported relative to the elongate channel 1102 and alternative anvils are further described herein. The elongate channel 1102/staple cartridge assembly 1110 and the anvil 1130 may also be referred to as “jaws”. The interchangeable surgical tool assembly 1000 may further include an articulation joint 1200 and an articulation lock which can be configured to releasably hold the end effector 1100 in a desired articulated position about an articulation axis B-B which is transverse to a shaft axis SA. Details regarding the construction and operation of the articulation lock may be found in in U.S. patent application Ser. No. 13/803,086, entitled ARTICULATABLE SURGICAL INSTRUMENT COMPRISING AN ARTICULATION LOCK, published as U.S. Patent Application Publication No. 2014/0263541, the entire disclosure of which is hereby incorporated by reference herein. Additional details concerning the articulation lock may also be found in U.S. patent application Ser. No. 15/019,196, filed Feb. 9, 2016, entitled SURGICAL INSTRUMENT ARTICULATION MECHANISM WITH SLOTTED SECONDARY CONSTRAINT, the entire disclosure of which is hereby incorporated by reference herein.

The tool assembly is further described in U.S. Patent Publication No. 2018/0168584, entitled SURGICAL STAPLING INSTRUMENTS AND STAPLE-FORMING ANVILS, filed Dec. 21, 2016, which is incorporated by reference herein its entirety.

Many of the surgical instrument systems described herein are motivated by an electric motor; however, the surgical instrument systems described herein can be motivated in any suitable manner. In various instances, the surgical instrument systems described herein can be motivated by a manually-operated trigger, for example. In certain instances, the motors disclosed herein may comprise a portion or portions of a robotically controlled system. Moreover, any of the end effectors and/or tool assemblies disclosed herein can be utilized with a robotic surgical instrument system. U.S. patent application Ser. No. 13/118,241, entitled SURGICAL STAPLING INSTRUMENTS WITH ROTATABLE STAPLE DEPLOYMENT ARRANGEMENTS, issued as U.S. Pat. No. 9,072,535, for example, discloses several examples of a robotic surgical instrument system in greater detail and is incorporated by reference herein in its entirety.

The surgical instrument systems described herein have been described in connection with the deployment and deformation of staples; however, the embodiments described herein are not so limited. Various embodiments are envisioned which deploy fasteners other than staples, such as clamps or tacks, for example. Moreover, various embodiments are envisioned which utilize any suitable means for sealing tissue. For instance, an end effector in accordance with various embodiments can comprise electrodes configured to heat and seal the tissue. Also, for instance, an end effector in accordance with certain embodiments can apply vibrational energy to seal the tissue.

Various embodiments described herein are described in the context of linear end effectors and/or linear fastener cartridges. Such embodiments, and the teachings thereof, can be applied to non-linear end effectors and/or non-linear fastener cartridges, such as, for example, circular and/or contoured end effectors. For example, various end effectors, including non-linear end effectors, are disclosed in U.S. patent application Ser. No. 13/036,647, filed Feb. 28, 2011, entitled SURGICAL STAPLING INSTRUMENT, published as U.S. Patent Application Publication No. 2011/0226837, issued as U.S. Pat. No. 8,561,870, which is hereby incorporated by reference in its entirety. Additionally, U.S. patent application Ser. No. 12/893,461, filed Sep. 29, 2012, entitled STAPLE CARTRIDGE, published as U.S. Patent Application Publication No. 2012/0074198, is hereby incorporated by reference in its entirety. U.S. patent application Ser. No. 12/031,873, filed Feb. 15, 2008, entitled END EFFECTORS FOR A SURGICAL CUTTING AND STAPLING INSTRUMENT, issued as U.S. Pat. No. 7,980,443, is also hereby incorporated by reference in its entirety. U.S. Pat. No. 8,393,514, entitled SELECTIVELY ORIENTABLE IMPLANTABLE FASTENER CARTRIDGE, which issued on Mar. 12, 2013, is also hereby incorporated by reference in its entirety.

Although various devices have been described herein in connection with certain embodiments, modifications and variations to those embodiments may be implemented. Particular features, structures, or characteristics may be combined in any suitable manner in one or more embodiments. Thus, the particular features, structures, or characteristics illustrated or described in connection with one embodiment may be combined in whole or in part, with the features, structures or characteristics of one or more other embodiments without limitation. Also, where materials are disclosed for certain components, other materials may be used. Furthermore, according to various embodiments, a single component may be replaced by multiple components, and multiple components may be replaced by a single component, to perform a given function or functions. The foregoing description and following claims are intended to cover all such modification and variations.

The devices disclosed herein can be designed to be disposed of after a single use, or they can be designed to be used multiple times. In either case, however, a device can be reconditioned for reuse after at least one use. Reconditioning can include any combination of the steps including, but not limited to, the disassembly of the device, followed by cleaning or replacement of particular pieces of the device, and subsequent reassembly of the device. In particular, a reconditioning facility and/or surgical team can disassemble a device and, after cleaning and/or replacing particular parts of the device, the device can be reassembled for subsequent use. Those skilled in the art will appreciate that reconditioning of a device can utilize a variety of techniques for disassembly, cleaning/replacement, and reassembly. Use of such techniques, and the resulting reconditioned device, are all within the scope of the present application.

The devices disclosed herein may be processed before surgery. First, a new or used instrument may be obtained and, when necessary, cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, and/or high-energy electrons. The radiation may kill bacteria on the instrument and in the container. The sterilized instrument may then be stored in the sterile container. The sealed container may keep the instrument sterile until it is opened in a medical facility. A device may also be sterilized using any other technique known in the art, including but not limited to beta radiation, gamma radiation, ethylene oxide, plasma peroxide, and/or steam.

While this invention has been described as having exemplary designs, the present invention may be further modified within the spirit and scope of the disclosure. This application is therefore intended to cover any variations, uses, or adaptations of the invention using its general principles.

What is claimed is:

1. A surgical stapling assembly, comprising:
a first jaw and a second jaw, wherein at least one of the first jaw and the second jaw are movable between an open configuration and a closed configuration;
a fastener cartridge, comprising:
a deck;
a longitudinal slot defining a first cartridge side and a second cartridge side;
staple cavities defined in the deck, wherein the staple cavities comprise a longitudinally-oriented staple cavity and an obliquely-oriented staple cavity on the first cartridge side;
staples removably stored in the staple cavities, wherein the staples comprise a longitudinally-oriented staple in the longitudinally-oriented staple cavity and an obliquely-oriented staple in the obliquely-oriented staple cavity; and
drivers movably supporting the staples, wherein the drivers comprise a first driver supporting the longitudinally-oriented staple and the obliquely-oriented staple;
an anvil comprising an anvil surface comprising staple forming pockets; and
a proximal tissue stop defining a proximal-most tissue plane extending through the deck and the anvil surface when the first jaw and the second jaw are in the closed configuration, wherein the longitudinally-oriented staple and the obliquely-oriented staple are positioned proximal to the proximal-most tissue plane.

2. The surgical stapling assembly of claim 1, further comprising a firing member configured to translate along the longitudinal slot and sequentially fire the staples from the fastener cartridge during a firing stroke.

3. The surgical stapling assembly of claim 1, wherein the staple forming pockets comprise:
a longitudinally-oriented pocket corresponding to the longitudinally-oriented staple; and
an obliquely-oriented pocket corresponding to the obliquely-oriented staple.

4. The surgical stapling assembly of claim 3, wherein, in the closed configuration, the longitudinally-oriented staple is aligned with the longitudinally-oriented pocket and the obliquely-oriented staple is aligned with the obliquely-oriented pocket.

5. The surgical stapling assembly of claim 3, wherein the longitudinally-oriented pocket is configured to form the longitudinally-oriented staple into a planar configuration, and wherein the obliquely-oriented pocket is configured to form the obliquely-oriented staple into a non-planar configuration.

6. The surgical stapling assembly of claim 3, wherein the longitudinally-oriented pocket comprises:
a first cup comprising a first entry ramp and a first exit ramp;
a second cup comprising an second entry ramp and a second exit ramp; and
a longitudinal pocket centerline extending between the first entry ramp and the second entry ramp, wherein the first exit ramp and the second exit ramp extends along the longitudinal pocket centerline.

7. The surgical stapling assembly of claim 6, wherein the obliquely-oriented pocket comprises:
a third cup comprising a third entry ramp and a third exit ramp;
a fourth cup comprising an fourth entry ramp and a fourth exit ramp; and
an oblique pocket centerline extending between the third entry ramp and the fourth entry ramp, wherein the third exit ramp and the fourth exit ramp diverge from the oblique pocket centerline.

8. The surgical stapling assembly of claim 1, wherein the staple cavities are arranged to form rows of cavities in the fastener cartridge, wherein the rows of cavities comprise:
an inner row comprising a series of longitudinally-oriented staple cavities comprising the longitudinally-oriented staple cavity; and
an outer row comprising a series of obliquely-oriented staple cavities comprising the obliquely-oriented staple cavity.

9. The surgical stapling assembly of claim 1, wherein the proximal tissue stop comprises opposing tissue stops extending from the anvil toward the deck.

10. The surgical stapling assembly of claim 9, wherein the opposing tissue stops comprise upright distal-facing edges, and wherein the upright distal-facing edges define the proximal-most tissue plane.

11. A surgical stapling assembly, comprising:
a fastener cartridge, comprising:
a deck;
a longitudinal slot defining a first cartridge side and a second cartridge side;
staple cavities defined in the deck, wherein the staple cavities comprise a series of longitudinally-oriented staple cavities in a first row on the first cartridge side and a series of obliquely-oriented staple cavities in a second row on the first cartridge side;
staples removably stored in the staple cavities, wherein the staples comprise a first longitudinally-oriented staple and a first obliquely-oriented staple; and
drivers movably supporting the staples, wherein the drivers comprise a triple driver movably supporting the first longitudinally-oriented staple and the first obliquely-oriented staple; and
a proximal tissue stop defining a proximal-most tissue plane extending through the deck, wherein the first longitudinally-oriented staple and the first obliquely-oriented staple are positioned proximal to the proximal-most tissue plane.

12. The surgical stapling assembly of claim 11, wherein the staple cavities further comprise a second series of obliquely-oriented staple cavities in a third row on the first cartridge side, wherein the second row is intermediate the first row and the third row.

13. The surgical stapling assembly of claim 12, wherein the first row comprises an inner row adjacent to the longitudinal slot.

14. The surgical stapling assembly of claim 11, further comprising a first jaw and a second jaw, wherein at least one of the first jaw and the second jaw are movable between an open configuration and a closed configuration.

15. The surgical stapling assembly of claim 14, wherein the fastener cartridge is mounted to the first jaw.

16. The surgical stapling assembly of claim 15, further comprising an anvil mounted to the second jaw, wherein the anvil comprises staple forming pockets.

17. The surgical stapling assembly of claim 11, further comprising a firing member configured translate along the longitudinal slot and sequentially fire the staples from the fastener cartridge during a firing stroke.

18. A surgical stapling assembly, comprising:
a first jaw and a second jaw, wherein at least one of the first jaw and the second jaw are movable between an open configuration and a closed configuration;
a fastener cartridge, comprising a deck, a longitudinal slot defining a first cartridge side and a second cartridge side, first staple cavities defined in the deck in a first cavity row on the first cartridge side, second staple cavities defined in the deck in a second cavity row on the first cartridge side, and staples removably stored in the first staple cavities and the second staple cavities;
a firing member configured to translate along the longitudinal slot to fire the staples;
an anvil, comprising:
an anvil surface;
first forming pockets defined in the anvil surface, wherein the first forming pockets are configured to form the staples removably stored in the first staple cavities into a planar staple configuration; and
second forming pockets defined in the anvil surface, wherein the second forming pockets are configured to form the staples removably stored in the second staple cavities into a nonplanar staple configuration; and
a proximal tissue stop defining a proximal-most tissue plane extending through the deck and the anvil surface, wherein at least one first forming pocket and at least one second forming pocket are proximal to the proximal-most tissue plane.

19. The surgical stapling assembly of claim 18, wherein each first forming pocket comprises:
a first cup comprising a first entry ramp and a first exit ramp;
a second cup comprising an second entry ramp and a second exit ramp; and
a first pocket centerline extending between the first entry ramp and the second entry ramp, wherein the first exit ramp and the second exit ramp extends along the first pocket centerline, and wherein each second forming pocket comprises:
a third cup comprising a third entry ramp and a third exit ramp;
a fourth cup comprising an fourth entry ramp and a fourth exit ramp; and
a second pocket centerline extending between the third entry ramp and the fourth entry ramp, wherein the third exit ramp and the fourth exit ramp diverge from the second pocket centerline.

20. The surgical stapling assembly of claim 18, wherein the first pocket centerlines are oriented parallel to the longitudinal slot, and wherein the second pocket centerlines are obliquely-oriented relative to the longitudinal slot.
